(19) United States
(12) Patent Application Publication
(10) Pub. No.: US 2009/0306373 A1
(43) Pub. Date: Dec. 10, 2009

(54) 4,7-DIHYDROTHIENO[2,3-B]PYRIDINE COMPOUNDS AND PHARMACEUTICAL COMPOSITIONS

(76) Inventors: Gabriel G. Gamber, Marlborough, MA (US); Rishi K. Jain, Cambridge, MA (US); Gary M. Ksander, Amherst, NH (US); Leslie W. McQuire, Cambrige, MA (US); Lawrence S. Melvin, Longmont, CO (US); Moo J. Sung, Belmont, MA (US)

Correspondence Address: HARNESS, DICKEY, & PIERCE, P.L.C, 7700 Bonhomme, Suite 400, ST. LOUIS, MO, 63105, US

(21) Appl. No.: 12/158,588

(22) PCT Filed: Dec. 19, 2006
(86) PCT No.: PCT/US06/62345

§ 371 Date: May 15, 2009

Related U.S. Application Data
(60) Provisional application No. 60/752,145, filed on Dec. 20, 2005.

(51) Int. Cl. C07D 495/04

(52) U.S. Cl. 544/127; 546/114

(57) ABSTRACT

This invention relates generally to compounds and pharmaceutical compositions for the treatment of myosin heavy chain (MyHC)-mediated conditions, and in particular, cardiovascular conditions.

### CROSS-REFERENCE TO CO-FILED APPLICATIONS

This patent application claims priority to U.S. Provisional Patent Application No. 60/752,145 (filed Dec. 20, 2005). This patent application is also co-filed with commonly assigned International Patent Application No. PCT/US2006/______ entitled “Methods for the Treatment of Myosin Heavy Chain-Mediated Conditions Using 4,7-Dihydrothieno[2,3-b]Pyridine Compounds” (filed Dec. 19, 2006) (attorney reference 8493-000046/WO/POA). Both applications are incorporated by reference in their entirety into this patent application.

### FIELD OF THE INVENTION

This invention relates generally to compounds and pharmaceutical compositions for the treatment of myosin heavy chain (MyHC)-mediated conditions, and in particular, cardiovascular conditions.

### BACKGROUND OF THE INVENTION

Heart failure is a pathophysiological state in which the heart fails to pump blood at a rate commensurate with the requirements of the metabolizing tissues of the body. It is caused in most cases, about 95% of the time, by myocardial failure. The contractile proteins of the heart lie within the muscle cells, called myocytes, which constitute about 75% of the total volume of the myocardium. The two major contractile proteins are the thin actin filament and the thick myosin filament. Each myosin filament contains two heavy chains and four light chains. The bodies of the heavy chains are intertwined, and each heavy chain ends in a head. Each lobe of the bi-lobed myosin head has an ATP-binding pocket, which has in close proximity the myosin ATPase activity that breaks down ATP.

The velocity of cardiac muscle contraction is controlled by the degree of ATPase activity in the head regions of the myosin molecules. The major determinant of myosin ATPase activity and, therefore, of the speed of muscle contraction, is the relative amount of the two myosin heavy chain isomers, alpha myosin heavy chain (alpha-MyHC) and beta myosin heavy chain (beta-MyHC). The alpha-MyHC isoform has approximately 2-3 times more enzymatic activity than the beta-MyHC isoform and, consequently, the velocity of cardiac muscle shortening is related to the relative percentages of each isoform. For example, adult rodent ventricular myocardium has approximately 80-90% alpha-MyHC, and only 10-20% beta-MyHC, which explains why its myosin ATPase activity is 3-4 times greater than bovine ventricular myocardium, which contains 80-90% beta-MyHC.

When ventricular myocardial hypertrophy or heart failure is created in rodent models, a change occurs in the expression of MyHC isoforms, with alpha-MyHC decreasing and beta-MyHC increasing. These “isoform switches” reduce the contractility of the hypertrophied rodent ventricle, ultimately leading to myocardial failure. This pattern of altered gene expression has been referred to as a reversion to a “fetal” expression pattern because during fetal and early neonatal development beta-MyHC also dominates in rodent ventricular myocardium.

It has been shown that myocardial function declines with age in animals. Cellular and molecular mechanisms that account for age-associated changes in myocardial performance have been studied largely in rodents. Among other changes, marked shifts in MyHC occur in rodents, i.e., the beta isoform becomes predominant in senescent rats. Steady-state mRNA levels for alpha-MyHC and beta-MyHC parallel the age-associated changes in the MyHC proteins. The myosin ATPase activity declines with the decline in alpha-MyHC content, and the altered cellular profile results in a contraction that exhibits a reduced velocity and a prolonged time course.

Human atrial myocardium most likely undergoes similar isoform switches with hypertrophy or failure. Several studies have examined this issue in autopsy cases, but did not find biologically significant expression of the alpha-MyHC isoform in putatively normal hearts. Since there was thought to be no significant expression of alpha-MyHC in normal hearts, a down-regulation in alpha-MyHC was not thought to be a possible basis for myocardial failure in humans. There was one early report that the amount of alpha-MyHC, although extremely small to begin with, was reduced in failing human myocardium. (Bouvagnet, 1989). However, more recent reports have shown the existence of appreciable levels of α-MyHC in the human heart at both the mRNA and protein level. At the mRNA level, 23-34% of the total ventricular mRNA is derived from alpha-MyHC (Lowes et al., 1997; Nakao et al., 1997), while approximately 1-10% of the total myosin protein content is alpha-MyHC (Miyata et al., 2000; Reiser et al., 2001). Changes in MyHC isoform content within their ranges are sufficient to explain the decrease in myosin or myofibrillar ATPase activity in the failing human heart (Hajjar et al., 1992; Pagani et al., 1988).

Data generated in the 1990's suggested that beta-myosin heavy chain mutations may account for approximately 30-40% percent of cases of familial hypertrophic cardiomyopathy (Watkins et al., 1992; Schwartz et al., 1995; Marian and Roberts, 1995; Thierfelder et al., 1994; Watkins et al., 1995). A patient with no family history of hypertrophic cardiomyopathy presented with late-onset cardiac hypertrophy of unknown etiology, and was shown to have a mutation hi α-MyHC (Niimura et al., 2002). Two important studies have shown even more convincingly the important role of the MyHC isoforms in cardiovascular disease. Lowes et al. (2002) showed that using beta blockers to treat dilated cardiomyopathy led to increased levels of alpha-MyHC and decreased levels of beta-MyHC that directly corresponded to improvement in disease state. In fact, the changes in alpha-MyHC noted in those studies was the only factor shown to correlate with improvement in cardiac function. Equally convincingly, Abraham et al. (2002) have shown that human myosin heavy chain isoform changes directly contribute to disease progression in dilated cardiomyopathy. These studies show the importance and need for an agent that can alter, if not reverse, the isoform switching that occurs in the MyHC isoforms in cardiovascular disease.

Cemova, L. et al. RTU Zinatniskie Raksti, Serija 1: Mataeriazinatne un Lietiska Kimya, 6:106-08, 2003 discusses the synthesis of 3-Amino-2-benzoyl-5-ethoxycarbonyl-4-phenyl-6-methyl-4,7-dihydrothieno[2,3-b]pyridine.

Dyachenko, V. D. et al., Chemistry of heterocyclic Compounds, 33(5):577-82, 1997 discusses thienyl substituted 1,4-dihydropyridine as being pharmacologically active.

Sharanin, Y. A. et al., Zhurnal Organicheskoi Khimii, 22(12):2600-09, 1986 discusses the preparation of 3-amino-4,7-dihdyrothieno[2,3-b]pyridine through a cyclization reaction.

WO 2005/37779 discusses compounds used for prophylaxis and therapy of acute neuronal diseases, in particular ischemia-caused cerebral damages after a ischemic or hemorrhagic stroke, craniocerebral trauma, cardiac arrest, myocardial infarct or as a consequence of heart surgery.

US 2005/0124633 discusses substituted 1,4-dihydropyridine compounds, including pure “S” enantiomeric forms which provide for elevation of alpha-MyHC mRNA levels and their use for treating heart failure.

US 2005/0124634 discusses substituted 1,4-dihydropyridine compounds, including pure “R” enantiomeric forms which provide for elevation of alpha-MyHC mRNA levels and their use for treating heart failure.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

### SUMMARY OF THE INVENTION

The present invention relates to compounds (and salts thereof) and pharmaceutical compositions for the treatment of myosin heavy chain (MyHC)-mediated conditions, and in particular, cardiovascular conditions or heart failure.

In one embodiment, compounds of Formula I (and salts thereof) are provided:

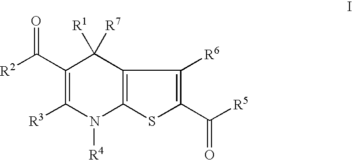

wherein:
R¹ is selected from the group consisting of phenyl, pyridinyl, cyclohexyl, benzodioxolyl, pyrazolyl, and naphthalenyl, wherein:
the phenyl, pyridinyl, cyclohexyl, pyrazolyl, and naphthalenyl are optionally substituted with one or more substituents independently selected from the group consisting of halogen, carboxy, alkoxy, hydroxyl, and alkyl, wherein:
   the alkyl portions of such substituents optionally are substituted with a substituent selected from the group consisting of alkoxycarbonylamino, halogen, carboxy, alkylcarbonylamino, amino, alkylamino, and alkoxyamino, wherein:
      the amino is optionally substituted with one or two substituents independently selected from the group consisting of alkyl, alkenyl, alkynyl, alkylcarbonyl, and alkoxycarbonyl;

R² is selected from the group consisting of monocyclic carbocyclyl, monocyclic heterocyclyl, naphthalenyl, alkyl, alkenyl, alkynyl, cycloalkyl, cycloalkyloxy, amino, alkoxy and phenyl, wherein:
the amino is optionally substituted with one or two substituents independently selected from the group consisting of alkyl, alkenyl, alkynyl, and cycloalkyl;
the alkoxy is optionally substituted with a substituent selected from the group consisting of alkyl, alkenyl, alkynyl, alkoxy, amino, monocyclic heterocyclyl, and monocyclic carbocyclyl, wherein:
   the amino is optionally substituted with one or two substituents independently selected from the group consisting of carboxyalkoxyalkylcarbonyl, carboxyallkoxycarbonyl, alkylcarbonyl, alkoxycarbonyl, phenylalkyl, R⁸-alkylcarbonyl, and R⁸-carbonylaminoalkylcarbonyl, and
   the heterocyclyl and carbocyclyl are optionally substituted with one or more substituents independently selected from the group consisting of alkyl, alkenyl, alkynyl, and alkoxy; and

R³ is selected from the group consisting of alkyl, alkenyl, alkynyl, phenylalkyl, alkoxyalkyl, haloalkyl, cycloalkyl, cycloalkenyl, phenyl, halophenyl, and alkoxy;

R⁴ is selected from the group consisting of hydrogen, alkyl, alkenyl, alkyl, cycloalkyl, and alkoxyalkoxyalkyl;

R⁵ is selected from the group consisting of phenyl, pyridinyl, and benzodioxolyl, wherein:
the phenyl and pyridinyl are optionally ortho-mono-substituted, meta-mono-substituted, di-substituted, or tri-substituted with substituent(s) independently selected from the group consisting of halo-en, azido, nitro, carboxy, cyano, alkyl, alkenyl, alkynyl, hydroxyl, alkoxy, thiol, alkylthio, haloalkyl, alkylcarbonyl, alkoxycarbonyl, alkoxycarbonylalkyl, and amino, wherein:
   the amino is optionally substituted with one or two substituents independently selected from the group consisting of alkoxycarbonyl and alkylcarbonyl;

R⁶ is selected from the group consisting of hydrogen and amino;

R⁷ is selected from the group consisting of hydrogen, alkyl, alkenyl, and alkynyl;

R⁸ is selected from the group consisting of

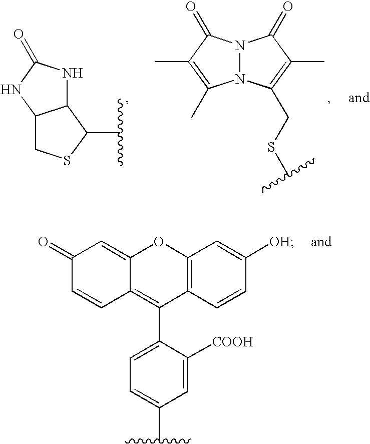

when compounds of Formula I are racemic, the following are excluded:

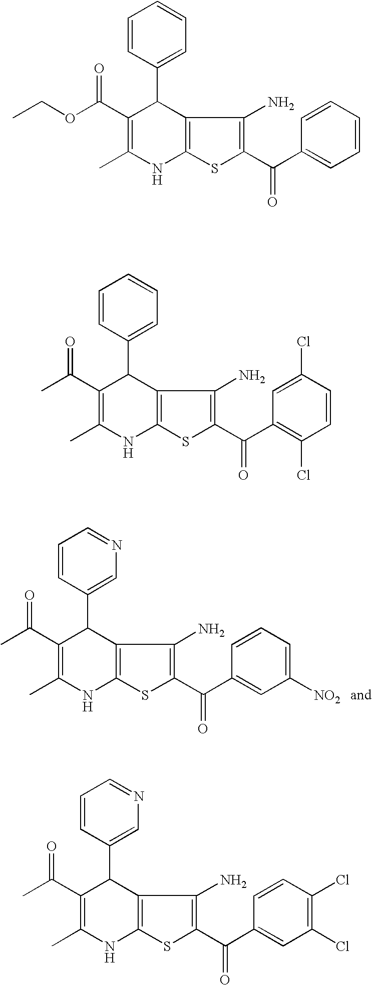

In another embodiment, compounds of the Formula II (and salts thereof) are provided:

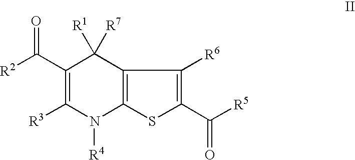

wherein:

R¹ is selected from the group consisting of monocyclic carbocyclyl, monocyclic heterocyclyl, naphthalenyl, and benzodioxolyl, wherein:
the carbocyclyl, heterocyclyl, and naphthalenyl are optionally substituted with one or more substituents independently selected from the group consisting of carboxy, alkyl, alkenyl, alkynyl, cycloalkyl, halogen, thiol, alkylthio, hydroxy, alkoxy, cyano, azido, nitro, and amino, wherein:
   the alkyl portions of such substituents are optionally substituted with a substituent selected from the group consisting of thiol, alkoxy, halogen, and alkoxycarbonylamino; and
   the amino is optionally substituted with one or two substituents selected from the group consisting of alkyl, alkenyl, alkynyl, alkylcarbonyl, and alkoxycarbonyl;

R² is selected from the group consisting of monocyclic carbocyclyl, monocyclic heterocyclyl, naphthalenyl, alkyl, alkenyl, alkynyl, alkoxy, cycloalkyloxy, and amino, wherein:
the amino is optionally substituted with one or two substituents independently selected from the group consisting of alkyl, alkenyl, alkynyl, and cycloalkyl; and
the alkoxy is optionally substituted with a substituent selected from the group consisting of alkyl, alkenyl, alkynyl, alkoxy, amino, N-morpholinyl, and N-methylpyrrolidinyl, wherein:
   the amino is optionally substituted with one or two substituents independently selected from the group consisting of carboxyalkoxyalkylcarbonyl, carboxyalkoxycarbonyl, carboxyalkylcarbonyl, alkylcarbonyl, alkoxycarboonyl, phenylalkyl, R⁸-alkylcarbonyl, and R⁸-carbonylaminoalkylcarbonyl;

R³ is selected from the group consisting of C₂-C₈-alkyl, alkenyl, alkynyl, alkoxy, cycloalkyl, cycloalkenyl, and phenyl wherein:
the alkyl portions of such substituents are optionally substituted with a substituent selected from the group consisting of phenyl, alkoxy, and halogen; and
the phenyl is optionally substituted with one or more substituents independently selected from the group consisting of halogen, alkyl, alkenyl, alkyl, alkoxy, and amino;

R⁴ is selected from the group consisting of hydrogen, alkyl, alkenyl, alkynyl cycloalkyl, and alkoxyalkoxyalkyl;

R⁵ is selected from the group consisting of phenyl, pyridinyl, and benzodioxolyl, wherein:
the phenyl and pyridinyl are optionally substituted with one or more substituents independently selected from the group consisting of halogen, nitro, azido, carboxy, cyano, alkyl, alkenyl, alkynyl, hydroxy, alkoxy, thiol, alkylthio, haloalkyl, alkylcarbonyl, alkoxycarbonyl, and amino, wherein:
   the amino is optionally substituted with one or two substituents independently selected from the group consisting of alkoxycarbonyl, alkylcarbonyl, alkoxycarbonylaminoalkylcarbonyl, and aminoalkylcarbonyl;

R⁶ is selected from the group consisting of hydrogen and amino;

R⁷ is selected from the group consisting of hydrogen, alkyl, alkenyl, and alkynyl;

R⁸ is selected from the group consisting of

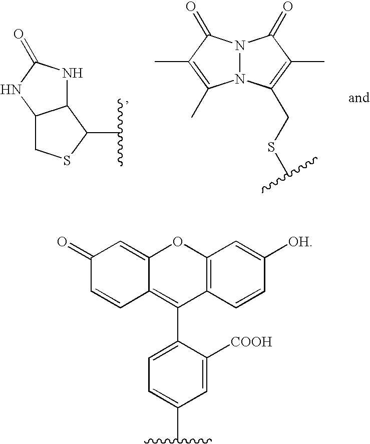

### DETAILED DESCRIPTION

This detailed description is intended to acquaint others skilled in the art with applicants' invention, its principles, and its practical application so that others skilled in the art may adapt and apply the invention in its numerous forms, as they may be best suited to the requirements of a particular use. This detailed description and its specific examples, while indicating embodiments of the present invention, are intended for purposes of illustration only. The present invention, therefore, is not limited to the embodiments described in this patent application, and may be variously modified.

The present invention provides 4,7-dihydrothieno[2,3-b]pyridine compounds and salts thereof (including all isomers thereof).

In one embodiment, the compounds correspond in structure to Formula I:

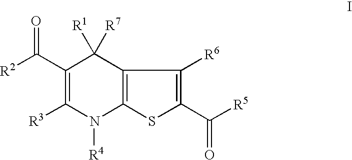

wherein:
R¹ is selected from the group consisting of phenyl, pyridinyl, cyclohexyl, benzodioxolyl, pyrazolyl, and naphthalenyl, wherein:
the phenyl, pyridinyl, cyclohexyl, pyrazolyl, and naphthalenyl are optionally substituted with one or more substituents independently selected from the group consisting of halogen, carboxy, alkoxy, hydroxyl, and alkyl, wherein:
   the alkyl portions of such substituents optionally are substituted with a substituent selected from the group consisting of alkoxycarbonylamino, halogen, carboxy, alkylcarbonylamino, amino, alkylamino, and alkoxyamino, wherein:
      the amino is optionally substituted with one or two substituents independently selected from the group consisting of alkyl, alkenyl, alkynyl, alkylcarbonyl, and alkoxycarbonyl;

R² is selected from the group consisting of monocyclic carbocyclyl, monocyclic heterocyclyl, naphthalenyl, alkyl, alkenyl, alkynyl, cycloalkyl, cycloalkyloxy, amino, alkoxy and phenyl, wherein:
the amino is optionally substituted with one or two substituents independently selected from the group consisting of alkyl, alkenyl, alkynyl, and cycloalkyl;
the alkoxy is optionally substituted with a substituent selected from the group consisting of alkyl, alkenyl, alkynyl, alkoxy, amino, monocyclic heterocyclyl, and monocyclic carbocyclyl, wherein:
   the amino is optionally substituted with one or two substituents independently selected from the group consisting of carboxyalkoxyalkylcarbonyl, carboxyalkoxycarbonyl, alkylcarbonyl, alkoxycarbonyl, phenylalkyl, R⁸-alkylcarbonyl, and R⁸-carbonylaminoalkylcarbonyl, and
   the heterocyclyl and carbocyclyl are optionally substituted with one or more substituents independently selected from the group consisting of alkyl, alkenyl, alkynyl, and alkoxy; and

R³ is selected from the group consisting of alkyl, alkenyl, alkynyl, phenylalkyl, alkoxyalkyl, haloalkyl, cycloalkyl, cycloalkenyl, phenyl, halophenyl, and alkoxy;

R⁴ is selected from the group consisting of hydrogen, alkyl, alkenyl, alkynyl, cycloalkyl, and alkoxyalkoxyalkyl;

R⁵ is selected from the group consisting of phenyl, pyridinyl, and benzodioxolyl, wherein:
the phenyl and pyridinyl are optionally ortho-mono-substituted, meta-mono-substituted, disubstituted, or tri-substituted with substituent(s) independently selected from the group consisting of halogen, azido, nitro, carboxy, cyano, alkyl, alkenyl, alkynyl, hydroxyl, alkoxy, thiol, alkylthio, haloalkyl, alkylcarbonyl, alkoxycarbonyl, alkoxycarbonylalkyl, and amino, wherein:
   the amino is optionally substituted with one or two substituents independently selected from the group consisting of alkoxycarbonyl and alkylcarbonyl;

R⁶ is selected from the group consisting of hydrogen and amino;

R⁷ is selected from the group consisting of hydrogen, alkyl, alkenyl, and alkynyl;

R⁸ is selected from the group consisting of

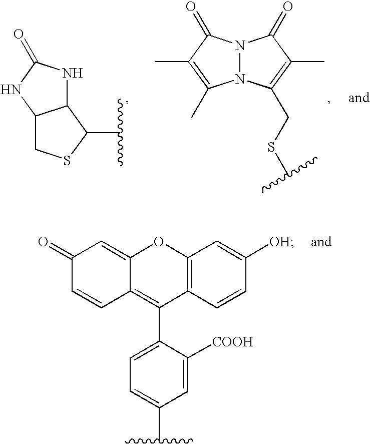

when compounds of Formula I are racemic, the following are excluded:

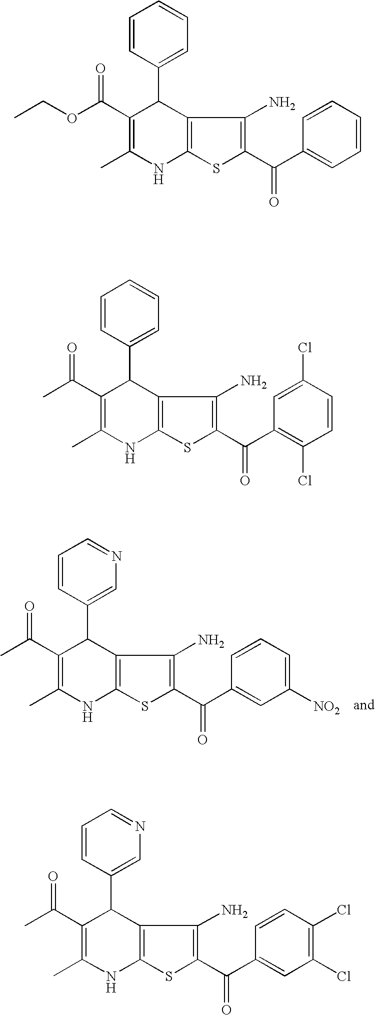

In some embodiments, R⁴ is hydrogen, R⁷ is selected from the group consisting of hydrogen mad alkyl, and R⁶ is amino.

In some embodiments, R⁴ is hydrogen, R⁷ is hydrogen and R⁶ is amino.

In some embodiments R¹ is selected from the group consisting of optionally substituted phenyl and optionally substituted pyridinyl; and R² is selected from the group consisting of alkyl, optionally substituted alkoxy, optionally substituted amino and optionally substituted phenyl; and R³ is selected from the group consisting of optionally substituted alkyl, cycloalkyl and optionally substituted phenyl; and R⁴ is selected from the group consisting of hydrogen and alkyl; and R⁵ is selected from the group consisting of optionally substituted phenyl and optionally substituted pyridinyl; and R⁶ is amino; and R⁷ is selected from the group consisting of hydrogen and methyl.

In some embodiments, R¹ is selected from the group consisting of optionally substituted phenyl and optionally substituted pyridinyl; and R² is selected from the group consisting of alkyl and optionally substituted alkoxy; and R³ is alkyl; and R⁴ is hydrogen; and R⁵ is selected from the group consisting of optionally substituted phenyl and optionally substituted pyridinyl; and R⁶ is amino; and R⁷ is hydrogen.

In some embodiments, R¹ is optionally substituted monocyclic carbocyclyl.

In some embodiments, R¹ is unsubstituted monocyclic carbocyclyl.

In some embodiments, R¹ is monocyclic carbocyclyl optionally substituted with one or more substituents selected from the group consisting of alkyl, alkoxy, hydrogen, nitro, and halogen.

In some embodiments, R¹ is monocyclic carbocyclyl optionally substituted with one or more halogen substituents.

In some embodiments, R¹ is optionally substituted monocyclic carbocyclyl, R⁴ is hydrogen, R⁷ is selected from the group consisting of hydrogen and allyl, and R⁶ is amino.

In some embodiments, R¹ is unsubstituted monocyclic carbocyclyl, R⁴ is hydrogen, R⁷ is selected from the group consisting of hydrogen and alkyl, and R⁶ is amino.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino.

In some embodiments, R¹ is optionally substituted monocyclic carbocyclyl and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is unsubstituted monocyclic carbocyclyl and R² and R³ are independently selected from the group consisting of alkyl, alkenyl, alkynyl, alkoxy, cycloalkyloxy, amino and phenyl.

In some embodiments, R¹ is monocyclic carbocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is monocyclic carbocyclyl optionally substituted with one or more halogen substituents and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is optionally substituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is unsubstituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is optionally substituted monocyclic carbocyclyl and R⁵ is phenyl optionally substituted with one or more substituents selected from the group consisting of halogen, cyano, nitro, and haloalkyl.

In some embodiments, R¹ is unsubstituted monocyclic carbocyclyl and R⁵ is phenyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen and R⁵ is phenyl optionally substituted with one or more substituents selected from the group consisting of halogen, cyano, nitro, and haloalkyl.

In some embodiments, R¹ is monocyclic carbocyclyl optionally substituted with one or more halogen substituents and R⁵ is phenyl optionally substituted with one or more substituents selected from the group consisting of halogen, cyano, nitro, and haloalkyl.

In some embodiments, R¹ is optionally substituted monocyclic carbocyclyl, R⁴ is hydrogen, R⁷ is selected from the group consisting of hydrogen and alkyl, and R⁶ is amino and R³ is phenyl optionally substituted with one or more substituents selected from the group consisting of halogen, cyano, nitro, and haloalkyl.

In some embodiments, R¹ is unsubstituted monocyclic carbocyclyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is optionally substituted monocyclic carbocyclyl and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted monocyclic carbocyclyl and R² is selected from the group consisting of optionally substituted alkoxy, alkyl, and optionally substituted amino and R³ is selected from the group consisting of alkyl, alkoxy, phenyl, and cycloalkyl and R¹ is phenyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic carbocyclyl optionally substituted with one or more halogen substituents and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R¹ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted monocyclic carbocyclyl and R⁵ is pyridinyl optionally substituted with one or more substituents selected from the group consisting of halogen, cyano, nitro, and haloalkyl.

In some embodiments, R¹ is unsubstituted monocyclic carbocyclyl and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more halogen substituents and R⁵ is pyridinyl optionally substituted with one or more substituents selected from the group consisting of halogen, cyano, nitro, and haloalkyl.

In some embodiments, R¹ is optionally substituted monocyclic carbocyclyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is unsubstituted monocyclic carbocyclyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is optionally substituted monocyclic carbocyclyl and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted monocyclic carbocyclyl and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl

In some embodiments, R¹ is monocyclic carbocyclyl optionally substituted with one or more halogen substituents and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted phenyl.

In some embodiments, R¹ is unsubstituted phenyl.

In some embodiments, R¹ is phenyl optionally substituted with a substituent selected from the group consisting of alkyl, alkoxy, hydrogen, nitro, and halogen.

In some embodiments, R¹ is phenyl optionally substituted with one or more halogen substituents.

In some embodiments, R¹ is optionally substituted phenyl and R² and R³ are independently selected from the group consisting of alkyl, alkenyl, alkynyl, alkoxy, cycloalkyloxy, amino and phenyl.

In some embodiments, R¹ is unsubstituted phenyl and R² and R³ are independently selected from the group consisting of alkyl, alkenyl, alkynyl, alkoxy, cycloalkyloxy, amino and phenyl.

In some embodiments, R¹ is phenyl optionally substituted with a substituent selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is phenyl optionally substituted with one or more halogen substituents and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is optionally substituted phenyl, R⁴ is hydrogen, R⁷ is selected from the group consisting of hydrogen and alkyl, and R⁶ is amino.

In some embodiments, R¹ is unsubstituted phenyl, R⁴ is hydrogen, R⁷ is selected from the group consisting of hydrogen and alkyl, and R⁶ is amino.

In some embodiments, R¹ is phenyl optionally substituted with a substituent selected from the group consisting of alkyl, alkoxy, hydrogen, nitro, and halogen, R⁴ is hydrogen, R⁷ is selected from the group consisting of hydrogen and alkyd and R⁶ is amino.

In some embodiments, R¹ is phenyl optionally substituted with one or more halogen substituents, R⁴ is hydrogen, R⁷ is selected from the group consisting of hydrogen and alkyl, and R⁶ is amino.

In some embodiments, R¹ is optionally substituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino mid R³ is selected from the group consisting of alkyl, alkoxy, phenyl, and cycloalkyl.

In some embodiments, R¹ is unsubstituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is phenyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is phenyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is optionally substituted phenyl and R¹ is phenyl optionally substituted with one or more substituents selected from the group consisting of halogen, cyano, nitro, and haloalkyl.

In some embodiments, R¹ is unsubstituted phenyl and R⁵ is phenyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen and R⁵ is phenyl optionally substituted with one or more substituents selected from the group consisting of halogen, cyano, nitro, and haloalkyl.

In some embodiments, R¹ is phenyl optionally substituted with one or more halogen substituents and

R⁵ is phenyl optionally substituted with one or more substituents selected from the group consisting of halogen, cyano, nitro, and haloalkyl.

In some embodiments, R¹ is optionally substituted phenyl and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted phenyl and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is phenyl optionally substituted with one or more halogen substituents and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted phenyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is unsubstituted phenyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is phenyl optionally substituted with one or more halogen substituents, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is optionally substituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen, R⁴ is hydrogen, R⁷ is selected from the group consisting of hydrogen and alkyl and R⁶ is amino and R³ is selected from the group consisting of optionally substituted alkoxy, alkyl, and optionally substituted amino and R³ is selected from the group consisting of alkyl, alkoxy, phenyl, and cycloakyl and R⁵ is phenyl optionally substituted with one or more substituents selected from the group consisting of halogen, cyano, nitro, and haloalkyl.

In some embodiments, R¹ is phenyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted phenyl and R⁵ is pyridinyl optionally substituted with one or more substituents selected from the group consisting of halogen, cyano, nitro, and haloalkyl.

In some embodiments, R¹ is unsubstituted phenyl and R⁵ is pyridinyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen and R⁵ is pyridinyl optionally substituted with one or more substituents selected from the group consisting of halogen, cyano, nitro, and haloalkyl.

In some embodiments, R¹ is phenyl optionally substituted with one or more halogen substituents and R⁵ is pyridinyl optionally substituted with one or more substituents selected from the group consisting of halogen, cyano, nitro, and haloalkyl.

In some embodiments, R¹ is optionally substituted phenyl and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted phenyl and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is phenyl optionally substituted with one or more halogen substituents and R² is selected from the group consisting of optionally substituted alkoxy, alkyl, and optionally substituted amino and R³ is selected from the group consisting of allyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted phenyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is unsubstituted phenyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is phenyl optionally substituted with a substituent selected from the group consisting of alkyd, alkoxy, hydrogen, nitro, and halogen, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is phenyl optionally substituted with one or more halogen substituents, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is optionally substituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is phenyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted phenyl; R² is alkyl; R³ is alkyl; R⁴ is hydrogen; R⁵ is unsubstituted phenyl; R⁶ is amino; and R⁷ is hydrogen.

In some embodiments, R¹ is unsubstituted phenyl; R² is alkyl; R³ is alkyl; R⁴ is hydrogen; R⁵ is unsubstituted pyridinyl; R⁶ is amino; and R⁷ is hydrogen.

In some embodiments, R¹ is unsubstituted phenyl; R² is alkyl; R³ is alkyl; R⁴ is hydrogen; R⁵ is phenyl substituted with one or more halogen; R⁶ is amino; and R⁷ is hydrogen.

In some embodiments, R¹ is unsubstituted phenyl; R² is alkyl; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is unsubstituted phenyl; R² is alkyl; R³ is alkyl; R⁴ is hydrogen; R⁵ is pyridinyl substituted with one or more halogen; R⁶ is amino; and R⁷ is hydrogen.

In some embodiments, R¹ is unsubstituted phenyl; R¹ is alkyl; R³ is alkyl; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is unsubstituted phenyl; R² is alkyl; R³ is alkoxy; R⁴ is hydrogen;

R⁵ is unsubstituted phenyl; R⁶ is amino; and R⁷ is hydrogen.

In some embodiments, R¹ is unsubstituted phenyl; R² is alkyl; R³ is alkoxy; R⁴ is hydrogen;

R⁵ is unsubstituted pyridinyl; R⁶ is amino; and R⁷ is hydrogen.

In some embodiments, R¹ is unsubstituted phenyl; R² is alkyl; R³ is alkoxy; R⁴ is hydrogen; R⁵ is phenyl substituted with one or more halogen; R⁶ is amino; and R⁷ is hydrogen.

In some embodiments, R¹ is unsubstituted phenyl; R² is alkyl; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is unsubstituted phenyl; R² is alkyl; R³ is alkoxy; R⁴ is hydrogen; R⁵ is pyridinyl substituted with one or more halogen; R⁶ is amino; and R⁷ is hydrogen.

In some embodiments, R¹ is unsubstituted phenyl; R² is alkyl; R³ is alkoxy; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is unsubstituted phenyl; R² is alkoxy; R³ is alkyl; R⁴ is hydrogen; R⁵ is unsubstituted phenyl; R⁶ is amino; and R⁷ is hydrogen.

In some embodiments, R¹ is unsubstituted phenyl; R² is alkoxy; R³ is alkyl; R⁴ is hydrogen; R⁵ is unsubstituted pyridinyl; R⁶ is amino; and R⁷ is hydrogen.

In some embodiments, R¹ is unsubstituted phenyl; R² is alkoxy; R³ is alkyl; R⁴ is hydrogen; R⁵ is phenyl substituted with one or more halogen; R⁶ is amino; and R⁷ is hydrogen.

In some embodiments, R¹ is unsubstituted phenyl; R² is alkoxy; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is unsubstituted phenyl; R² is alkoxy; R³ is alkyl; R⁴ is hydrogen; R⁵ is pyridinyl substituted with one or more halogen; R² is amino; and R⁷ is hydrogen.

In some embodiments, R¹ is unsubstituted phenyl; R² is alkoxy; R³ is alkyl; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is unsubstituted phenyl; R² is alkoxy; R³ is alkoxy; R⁴ is hydrogen; R⁵ is unsubstituted phenyl; R¹ is amino; and R⁷ is hydrogen.

In some embodiments, R¹ is unsubstituted phenyl; R² is alkoxy; R¹ is alkoxy; R⁴ is hydrogen; R⁵ is unsubstituted pyridinyl; R⁶ is amino; and R⁷ is hydrogen.

In some embodiments, R¹ is unsubstituted phenyl; R² is alkoxy; R³ is alkoxy; R⁴ is hydrogen; R⁵ is phenyl substituted with one or more halogen; R⁶ is amino; and R⁷ is hydrogen.

In some embodiments, R¹ is unsubstituted phenyl; R² is alkoxy; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is unsubstituted phenyl; R² is alkoxy; R³ is alkoxy; R⁴ is hydrogen; R⁵ is pyridinyl substituted with one or more halogen; R⁶ is amino; and R⁷ is hydrogen.

In some embodiments, R¹ is unsubstituted phenyl; R² is alkoxy; R³ is alkoxy; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is phenyl substituted with one or more halogen substituents; R² is alkyl; R³ is alkyl; R⁴ is hydrogen; R⁵ is unsubstituted phenyl; R⁶ is amino; and R⁷ is hydrogen.

In some embodiments, R¹ is phenyl substituted with one or more halogen substituents; R² is alkyl; R³ is alkyl; R⁴ is hydrogen; R⁵ is unsubstituted pyridinyl; R⁶ is amino; and R⁷ is hydrogen.

In some embodiments, R¹ is phenyl substituted with one or more halogen substituents; R² is alkyl; R³ is alkyl; R⁴ is hydrogen; R⁵ is phenyl substituted with one or more halogen; R⁶ is amino; and R⁷ is hydrogen.

In some embodiments, R¹ is phenyl substituted with one or more halogen substituents; R² is alkyl; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is phenyl substituted with one or more halogen substituents; R² is alkyl; R³ is alkyl; R⁴ is hydrogen; R⁵ is pyridinyl substituted with one or more halogen; R⁶ is amino; and R⁷ is hydrogen.

In some embodiments, R¹ is phenyl substituted with one or more halogen substituents; R² is alkyl; R³ is alkyl; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is unsubstituted phenyl; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is unsubstituted pyridinyl; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is phenyl substituted with one or more halogen; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is pyridinyl substituted with one or more halogen; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is unsubstituted phenyl; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is unsubstituted pyridinyl; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is phenyl substituted with one or more halogen; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is pyridinyl substituted with one or more halogen; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is unsubstituted phenyl; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is unsubstituted pyridinyl; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is phenyl substituted with one or more halogen; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is pyridinyl substituted with one or more halogen; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is pyridinyl substituted with one or more halogen substituents; R² is alkyl; R³ is alkyl; R⁴ is hydrogen; R⁵ is unsubstituted phenyl; R⁶ is amino; and R⁷ is hydrogen.

In some embodiments, R¹ is pyridinyl substituted with one or more halogen substituents; R² is alkyl; R³ is alkyl; R⁴ is hydrogen; R⁵ is unsubstituted pyridinyl; R⁶ is amino; and R⁷ is hydrogen.

In some embodiments, R¹ is pyridinyl substituted with one or more halogen substituents; R² is alkyl; R³ is alkyl; R⁴ is hydrogen; R⁵ is phenyl substituted with one or more halogen; R⁶ is amino; and R⁷ is hydrogen.

In some embodiments, R¹ is pyridinyl substituted with one or more halogen substituents; R² is alkyl; R³ is alkyl; R⁴ is hydrogen; R⁵ is phenyl substituted with one or more cyano, nitro or haloalkyl substituents; R⁶ is amino; and R⁷ is hydrogen

In some embodiments, R¹ is optionally substituted monocyclic heterocyclyl.

In some embodiments, R¹ is unsubstituted monocyclic heterocyclyl.

In some embodiments, R¹ is monocyclic heterocyclyl optionally substituted with one or more substituents selected from the group consisting of alkyl, alkoxy, hydrogen, nitro, and halogen.

In some embodiments, R¹ is monocyclic heterocyclyl optionally substituted with one or more halogen substituents.

In some embodiments, R¹ is optionally substituted monocyclic heterocyclyl, R⁴ is hydrogen, R⁷ is selected from the group consisting of hydrogen and alkyl, and R⁶ is amino.

In some embodiments, R¹ is unsubstituted monocyclic heterocyclyl, R⁴ is hydrogen, R⁷ is selected from the group consisting of hydrogen and alkyl, and R⁶ is amino.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino.

In some embodiments, R¹ is optionally substituted monocyclic heterocyclyl and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is unsubstituted monocyclic heterocyclyl and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is monocyclic heterocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is monocyclic heterocyclyl optionally substituted with one or more halogen substituents and R² is selected from the group consisting of optionally substituted alkoxy, alkyl, and optionally substituted amino and R¹ is selected from the group consisting of alkyl, alkoxy, phenyl, and cycloalkyl.

In some embodiments, R¹ is optionally substituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is unsubstituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is optionally substituted monocyclic heterocyclyl and R⁵ is phenyl optionally substituted with one or more substituents selected from the group consisting of halogen, cyano, nitro, and haloalkyl.

In some embodiments, R¹ is unsubstituted monocyclic heterocyclyl and R⁵ is phenyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen and R⁵ is phenyl optionally substituted with one or more substituents selected from the group consisting of halogen, cyano, nitro, and haloalkyl.

In some embodiments, R¹ is monocyclic heterocyclyl optionally substituted with one or more halogen substituents and R⁵ is phenyl optionally substituted with one or more substituents selected from the group consisting of halogen, cyano, nitro, and haloalkyl.

In some embodiments, R¹ is optionally substituted monocyclic heterocyclyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is unsubstituted monocyclic heterocyclyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is optionally substituted monocyclic heterocyclyl and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted monocyclic heterocyclyl and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic heterocyclyl optionally substituted with one or more halogen substituents and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted monocyclic heterocyclyl and R⁵ is pyridinyl optionally substituted with one or more substituents selected from the group consisting of halogen, cyano, nitro, and haloalkyl.

In some embodiments, R¹ is unsubstituted monocyclic heterocyclyl and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more halogen substituents and R⁵ is pyridinyl optionally substituted with one or more substituents selected from the group consisting of halogen, cyano, nitro, and haloalkyl.

In some embodiments, R¹ is optionally substituted monocyclic heterocyclyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is unsubstituted monocyclic heterocyclyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is optionally substituted monocyclic heterocyclyl and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted monocyclic heterocyclyl and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more halogen substituents and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted pyridinyl.

In some embodiments, R¹ is unsubstituted pyridinyl.

In some embodiments, R¹ is pyridinyl optionally substituted with a substituent selected from the group consisting of alkyl, alkoxy, hydrogen, nitro, and halogen.

In some embodiments, R¹ is pyridinyl optionally substituted with one or more halogen substituents.

In some embodiments, R¹ is optionally substituted pyridinyl and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is unsubstituted pyridinyl and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is pyridinyl optionally substituted with a substituent selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is pyridinyl optionally substituted with one or more halogen substituents and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is optionally substituted pyridinyl, R⁴ is hydrogen, R⁷ is selected from the group consisting of hydrogen and alkyl, and R⁶ is amino.

In some embodiments, R¹ is unsubstituted pyridinyl, R⁴ is hydrogen, R⁷ is selected from the group consisting of hydrogen and alkyl, and R⁶ is amino.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyl and R⁶ is amino.

In some embodiments, R¹ is pyridinyl optionally substituted with one or more halogen substituents, R⁴ is hydrogen, R⁷ is selected from the group consisting of hydrogen and alkyl, and R⁶ is amino.

In some embodiments, R¹ is optionally substituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is unsubstituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is pyridinyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl.

In some embodiments, R¹ is optionally substituted pyridinyl and R⁵ is phenyl optionally substituted with one or more substituents selected from the group consisting of halogen, cyano, nitro, and haloalkyl.

In some embodiments, R¹ is unsubstituted pyridinyl and R⁵ is phenyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen and R⁵ is phenyl optionally substituted with one or more substituents selected from the group consisting of halogen, cyano, nitro, and haloalkyl.

In some embodiments, R¹ is pyridinyl optionally substituted with one or more halogen substituents and R⁵ is phenyl optionally substituted with one or more substituents selected from the group consisting of halogen, cyano, nitro, and haloalkyl.

In some embodiments, R¹ is optionally substituted pyridinyl and R² is selected from the group consisting of optionally substituted alkoxy, alkyl, and optionally substituted amino and R³ is selected from the group consisting of alkyl, alkoxy, phenyl, and cycloaklyl and R⁵ is phenyl optionally substituted with one or more substituents selected from the group consisting of halogen, cyano, nitro, and haloalkyl.

In some embodiments, R¹ is unsubstituted pyridinyl and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is pyridinyl optionally substituted with one or more halogen substituents and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted pyridinyl, R⁴ is hydrogen, R⁷ is selected from the group consisting of hydrogen and alkyl, and R⁶ is amino and R⁵ is phenyl optionally substituted with one or more substituents selected from R⁶ group consisting of halogen, cyano, nitro, and haloalkyl.

In some embodiments, R¹ is unsubstituted pyridinyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyd and R⁶ is amino and R⁵ is phenyl optionally substituted with one or more substituents selected from the group consisting of halogen, cyano, nitro, and haloalkyl.

In some embodiments, R¹ is pyridinyl optionally substituted with one or more halogen substituents, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is optionally substituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is pyridinyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted pyridinyl and R⁵ is pyridinyl optionally substituted with one or more substituents selected from the group consisting of halogen, cyano, nitro, and haloalkyl.

In some embodiments, R¹ is unsubstituted pyridinyl and R⁵ is pyridinyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen and R⁵ is pyridinyl optionally substituted with one or more substituents selected from the group consisting of halogen, cyano, nitro, and haloalkyl.

In some embodiments, R¹ is pyridinyl optionally substituted with one or more halogen substituents and R⁵ is pyridinyl optionally substituted with one or more substituents selected from the group consisting of halogen, cyano, nitro, and haloalkyl.

In some embodiments, R¹ is optionally substituted pyridinyl and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted pyridinyl and R² is selected from the group consisting of optionally substituted alkoxy, alkyl, and optionally substituted amino and R³ is selected from the group consisting of alkyl, alkoxy, phenyl, and cycloalkyl and R¹ is pyridinyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is pyridinyl optionally substituted with one or more halogen substituents and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted pyridinyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is unsubstituted pyridinyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is pyridinyl optionally substituted with one or more halogen substituents, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is optionally substituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is pyridinyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted pyridinyl; R¹ is alkyl; R³ is alkyl; R⁴ is hydrogen; R⁵ is unsubstituted phenyl; R⁶ is amino; and R⁷ is hydrogen.

In some embodiments, R¹ is unsubstituted pyridinyl; R² is alkyl; R³ is alkyl; R⁴ is hydrogen; R⁵ is pyridinyl R⁶ is amino; and R¹⁷ is hydrogen.

In some embodiments, R¹ is unsubstituted pyridinyl; R² is alkyl; R³ is alkyl; R⁴ is hydrogen; R⁵ is phenyl substituted with one or more halogen; R⁶ is amino; and R⁷ is hydrogen.

In some embodiments, R¹ is unsubstituted pyridinyl; R² is alkyl; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is unsubstituted pyridinyl; R² is alkyl; R³ is alkyl; R⁴ is hydrogen; R⁵ is pyridinyl substituted with one or more halogen; R⁶ is amino; and R⁷ is hydrogen.

In some embodiments, R¹ is unsubstituted pyridinyl; R² is alkyl; R³ is alkyl; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is unsubstituted pyridinyl; R² is alkyl; R³ is alkoxy; R⁴ is hydrogen; R⁵ is unsubstituted phenyl; R⁶ is amino; and R⁷ is hydrogen.

In some embodiments, R¹ is unsubstituted pyridinyl; R² is alkyl; R³ is alkoxy; R⁴ is hydrogen; R⁵ is unsubstituted pyridinyl; R⁶ is amino; and R⁷ is hydrogen.

In some embodiments, R¹ is unsubstituted pyridinyl; R² is alkyl; R³ is alkoxy; R⁴ is hydrogen; R⁵ is phenyl substituted with one or more halogen; R⁶ is amino; and R⁷ is hydrogen.

In some embodiments, R¹ is unsubstituted pyridinyl; R² is alkyl; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is unsubstituted pyridinyl; R² is alkyl; R³ is alkoxy; R⁴ is hydrogen; R⁵ is pyridinyl substituted with one or more halogen; R⁶ is amino; and R⁷ is hydrogen.

In some embodiments, R¹ is unsubstituted pyridinyl; R² is alkyl; R³ is alkoxy; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is unsubstituted pyridinyl; R² is alkoxy; R³ is alkyl; R⁴ is hydrogen; R⁵ is unsubstituted phenyl; R⁶ is amino; and R⁷ is hydrogen.

In some embodiments, R¹ is unsubstituted pyridinyl; R² is alkoxy; R³ is alkyl; R⁴ is hydrogen; R⁵ is unsubstituted pyridinyl; R⁶ is amino; and R⁷ is hydrogen.

In some embodiments, R¹ is unsubstituted pyridinyl; R² is alkoxy; R³ is alkyl; R⁴ is hydrogen; R⁵ is phenyl substituted with one or more halogen; R⁶ is amino; and R⁷ is hydrogen.

In some embodiments, R¹ is unsubstituted pyridinyl; R² is alkoxy; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is unsubstituted pyridinyl; R² is alkoxy; R³ is alkyl; R⁴ is hydrogen; R⁵ is pyridinyl substituted with one or more halogen; R⁶ is amino; and R⁷ is hydrogen.

In some embodiments, R¹ is unsubstituted pyridinyl; R² is alkoxy; R³ is alkyl; R⁴ is hydrogen; R¹⁵ is pyridinyl substituted with one or more substituents selected from the group consisting of cyano, nitro and haloalkyl; R⁶ is amino; and R⁷ is hydrogen.

In some embodiments, R¹ is unsubstituted pyridinyl; R² is alkoxy; R³ is alkoxy; R⁴ is hydrogen; R⁵ is unsubstituted phenyl; R⁶ is amino; and R⁷ is hydrogen.

In some embodiments, R¹ is unsubstituted pyridinyl; R² is alkoxy; R³ is alkoxy; R⁴ is hydrogen; R⁵ is unsubstituted pyridinyl; R⁶ is amino; and R⁷ is hydrogen.

In some embodiments, R¹ is unsubstituted pyridinyl; R² is alkoxy; R³ is alkoxy; R⁴ is hydrogen; R⁵ is phenyl substituted with one or more halogen; R⁶ is amino; and R⁷ is hydrogen.

In some embodiments, R¹ is unsubstituted pyridinyl; R² is alkoxy; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is unsubstituted pyridinyl; R² is alkoxy; R³ is alkoxy; R⁴ is hydrogen; R⁵ is pyridinyl substituted with one or more halogen; R⁶ is amino; and R⁷ is hydrogen.

In some embodiments, R¹ is unsubstituted pyridinyl; R² is alkoxy; R³ is alkoxy; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is pyridinyl substituted with one or more halogen substituents; R² is alkyl; R³ is alkyl; R⁴ is hydrogen; R⁵ is pyridinyl substituted with one or more halogen; R⁶ is amino; and R⁷ is hydrogen.

In some embodiments, R¹ is pyridinyl substituted with one or more halogen substituents; R² is alkyl; R³ is alkyl; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is unsubstituted phenyl; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is unsubstituted pyridinyl; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is phenyl substituted with one or more halogen; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is pyridinyl substituted with one or more halogen; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is pyridinyl substituted with one or more halogen substituents; R² is alkoxy; R³ is alkyl; R⁴ is hydrogen; R⁵ is unsubstituted phenyl; R⁶ is amino; and R⁷ is hydrogen.

In some embodiments, R¹ is pyridinyl substituted with one or more halogen substituents; R² is alkoxy; R³ is alkyl; R⁴ is hydrogen; R⁵ is unsubstituted pyridinyl; R⁶ is amino; and R⁷ is hydrogen.

In some embodiments, R¹ is pyridinyl substituted with one or more halogen substituents; R² is alkoxy; R⁵ is alkyl; R⁴ is hydrogen; R⁵ is phenyl substituted with one or more halogen; R⁶ is amino; and R⁷ is hydrogen.

In some embodiments, R¹ is pyridinyl substituted with one or more halogen substituents; R² is alkoxy; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is pyridinyl substituted with one or more halogen substituents; R² is alkoxy; R³ is alkyl; R⁴ is hydrogen; R⁵ is pyridinyl substituted with one or more halogen; R⁶ is amino; and R⁷ is hydrogen.

In some embodiments, R¹ is pyridinyl substituted with one or more halogen substituents; R² is alkoxy; R³ is alkyl; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is pyridinyl substituted with one or more halogen substituents; R² is alkoxy; R³ is alkoxy; R⁴ is hydrogen; R⁵ is unsubstituted phenyl; R⁶ is amino; and R⁷ is hydrogen.

In some embodiments, R¹ is pyridinyl substituted with one or more halogen substituents; R² is alkoxy; R³ is alkoxy; R⁴ is hydrogen; R⁵ is unsubstituted pyridinyl; R⁶ is amino; and R⁷ is hydrogen.

In some embodiments, R¹ is pyridinyl substituted with one or more halogen substituents; R² is alkoxy; R³ is alkoxy; R⁴ is hydrogen; R⁵ is phenyl substituted with one or more halogen; R⁶ is amino; and R⁷ is hydrogen.

In some embodiments, R¹ is pyridinyl substituted with one or more halogen substituents; R² is alkoxy; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is pyridinyl substituted with one or more halogen substituents; R² is alkoxy; R³ is alkoxy; R⁴ is hydrogen; R¹ is pyridinyl substituted with one or more halogen; R⁶ is amino; and R⁷ is hydrogen.

In some embodiments, R¹ is pyridinyl substituted with one or more halogen substituents; R² is alkoxy; R³ is alkoxy; R⁴ is hydrogen; R⁵ is pyridinyl substituted with one or more substituents selected from the group consisting of cyano, nitro and haloalkyl; R⁶ is amino; and R⁷ is hydrogen.

In a second embodiment, compounds or salts thereof, wherein the compound corresponds in structure to Formula II:

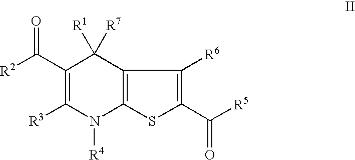

wherein:

R¹ is selected from the group consisting of monocyclic carbocyclyl, monocyclic heterocyclyl, naphthalenyl, and benzodioxolyl, wherein:
the carbocyclyl, heterocyclyl, and naphthalenyl are optionally substituted with one or more substituents independently selected from the group consisting of carboxy, alkyl, alkenyl, alkynyl, cycloalkyl, halogen, thiol, alkylthio, hydroxy, alkoxy, cyano, azido, nitro, and amino, wherein:
   the alkyl portions of such substituents are optionally substituted with a substituent selected from the group consisting of thiol, alkoxy, halogen, and alkoxycarbonylamino; and
   the amino is optionally substituted with one or two substituents selected from the group consisting of alkyl, alkenyl, alkynyl, alkylcarbonyl, and alkoxycarbonyl;

R² is selected from the group consisting of monocyclic carbocyclyl, monocyclic heterocyclyl, naphthalenyl, alkyl, alkenyl, alkynyl, alkoxy, cycloalkyloxy, and amino, wherein:
the amino is optionally substituted with one or two substituents independently selected from the group consisting of alkyl, alkenyl, alkynyl, and cycloalkyl; and
the alkoxy is optionally substituted with a substituent selected from the group consisting of alkyl, alkenyl, alkynyl, alkoxy, amino, N-morpholinyl, and N-methylpyrrolidinyl, wherein:
   the amino is optionally substituted with one or two substituents independently selected from the group consisting of carboxyalkoxyalkylcarbonyl, carboxyalkoxycarbonyl, carboxyalkylcarbonyl, alkylcarbonyl, alkoxycarbonyl, phenylalkyl, R⁸-alkylcarbonyl, and R⁸-carbonylaminoalkylcarbonyl;

R³ is selected from the group consisting of C₂-C₈-alkyl, alkenyl, alkynyl, alkoxy, cycloalkyl, cycloalkenyl, and phenyl wherein:
the alkyl portions of such substituents are optionally substituted with a substituent selected from the group consisting of phenyl, alkoxy, and halogen; and
the phenyl is optionally substituted with one or more substituents independently selected from the group consisting of halogen, alkyl, alkenyl, alkynyl, alkoxy, and amino;

R⁴ is selected from the group consisting of hydrogen, alkyl, alkenyl, alkynyl, cycloalkyl, and alkoxyalkoxyalkyl;

R⁵ is selected from the group consisting of phenyl, pyridinyl, and benzodioxolyl, wherein:
the phenyl and pyridinyl are optionally substituted with one or more substituents independently selected from the group consisting of halogen, nitro, azido, carboxy, cyano, alkyl, alkenyl, alkynyl, hydroxy, alkoxy, thiol, alkylthio, haloalkyl, alkylcarbonyl, alkoxycarbonyl, and amino, wherein:
   the amino is optionally substituted with one or two substituents independently selected from the group consisting of alkoxycarbonyl, alkylcarbonyl, alkoxycarbonylaminoalkylcarbonyl, and aminoalkylcarbonyl;

R⁶ is selected from the group consisting of hydrogen and amino;

R⁷ is selected from the group consisting of hydrogen, alkyl, alkenyl, and alkynyl;

R⁸ is selected from the group consisting of

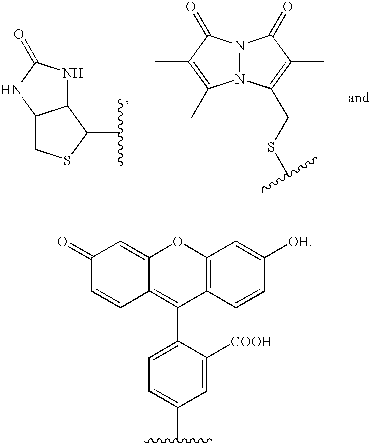

In some embodiments, R⁴ is hydrogen, R⁷ is selected from the group consisting of hydrogen and alkyl, and R⁶ is amino.

In some embodiments, R⁴ is hydrogen, R⁷ is hydrogen and R⁶ is amino.

In some embodiments, R¹ is selected from the group consisting of optionally substituted phenyl and optionally substituted pyridinyl; and R² is selected from the group consisting of C₂-C₈-alkyl, optionally substituted alkoxy, optionally substituted amino and optionally substituted phenyl; and R³ is selected from the group consisting of optionally substituted alkyl, cycloalkyl and optionally substituted phenyl; and R⁴ is selected from the group consisting of hydrogen and alkyl; and R⁵ is selected from the group consisting of optionally substituted phenyl and optionally substituted pyridinyl; and R⁶ is amino; and R⁷ is selected from the group consisting of hydrogen and methyl.

In some embodiments, R¹ is selected from the group consisting of optionally substituted phenyl and optionally substituted pyridinyl; and R² is selected from the group consisting of C₂-C₈-alkyl and optionally substituted alkoxy; and R³ is alkyl; and R⁴ is hydrogen; R⁵ is selected from the group consisting of optionally substituted phenyl and optionally substituted pyridinyl; and R⁶ is amino; and R⁷ is hydrogen.

In some embodiments, R¹ is optionally substituted monocyclic carbocyclyl.

In some embodiments, R¹ is unsubstituted monocyclic carbocyclyl.

In some embodiments, R¹ is monocyclic carbocyclyl optionally substituted with one or more substituents selected from the group consisting of alkyl, alkoxy, hydrogen, nitro, and halogen.

In some embodiments, R¹ is monocyclic carbocyclyl optionally substituted with one or more halogen substituents.

In some embodiments, R¹ is optionally substituted monocyclic carbocyclyl, R⁴ is hydrogen, R⁷ is selected from the group consisting of hydrogen and alkyl, and R⁶ is amino.

In some embodiments, R¹ is unsubstituted monocyclic carbocyclyl, R⁴ is hydrogen, R⁷ is selected from the group consisting of hydrogen and alkyl, and R⁶ is amino.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino. In some embodiments, R¹ is optionally substituted monocyclic carbocyclyl and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is unsubstituted monocyclic carbocyclyl and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is monocyclic carbocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is monocyclic carbocyclyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is optionally substituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is unsubstituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, pheny, and cycloalkyl.

In some embodiments, R³ is monocyclic carbocyclyl optionally substituted with one or more substituents selected from the group consisting of alkyl alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is optionally substituted monocyclic carbocyclyl and R⁵ is phenyl optionally substituted with one or more substituents selected from the group consisting of halogen, cyano, nitro, and haloalkyl.

In some embodiments, R¹ is unsubstituted monocyclic carbocyclyl and R⁵ is phenyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen and R⁵ is phenyl optionally substituted with one or more substituents selected from the group consisting of halogen, cyano, nitro, and haloalkyl.

In some embodiments, R¹ is monocyclic carbocyclyl optionally substituted with one or more halogen substituents and R⁵ is phenyl optionally substituted with one or more substituents selected from the group consisting of halogen, cyano, nitro, and haloalkyl.

In some embodiments, R¹ is optionally substituted monocyclic carbocyclyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is unsubstituted monocyclic carbocyclyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is optionally substituted monocyclic carbocyclyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted monocyclic carbocyclyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic carbocyclyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R³ is phenyl optionally substituted with one or more substituents selected from the group consisting of halogen, cyano, nitro, and haloalkyl.

In some embodiments, R¹ is unsubstituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted monocyclic carbocyclyl and R⁵ is pyridinyl optionally substituted with one or more substituents selected from the group consisting of halogen, cyano, nitro, and haloalkyl.

In some embodiments, R¹ is unsubstituted monocyclic carbocyclyl and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more halogen substituents and R⁵ is pyridinyl optionally substituted with one or more substituents selected from the group consisting of halogen, cyano, nitro, and haloalkyl.

In some embodiments, R¹ is optionally substituted monocyclic carbocyclyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is unsubstituted monocyclic carbocyclyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is optionally substituted monocyclic carbocyclyl and R² is selected from the group consisting of optionally substituted alkoxy, alkyl, and optionally substituted amino and R³ is selected from the group consisting of C₂-C₈-alkyl, alkoxy, phenyl, and cycloaklyl and R⁵ is pyridinyl optionally substituted with one or more substituents selected from the group consisting of halogen, cyano, nitro, and haloalkyl.

In some embodiments, R¹ is unsubstituted monocyclic carbocyclyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of C₂-C₈-alkyl, alkoxy, phenyl, and cycloaklyl and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₉-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted monocyclic carbocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic carbocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted phenyl.

In some embodiments, R¹ is unsubstituted phenyl.

In some embodiments, R¹ is phenyl optionally substituted with a substituent selected from the group consisting of alkyl, alkoxy, hydrogen, nitro, and halogen.

In some embodiments, R¹ is phenyl optionally substituted with one or more halogen substituents.

In some embodiments, R¹ is optionally substituted phenyl and R² and R³ are independently selected from the group consisting of alkyl, alkenyl, alkynyl, alkoxy, cycloalkyloxy, amino and phenyl.

In some embodiments, R¹ is unsubstituted phenyl and R² and R³ are independently selected from the group consisting of alkyl, alkenyl, alkynyl, alkoxy, cycloalkyloxy, amino and phenyl.

In some embodiments, R¹ is phenyl optionally substituted with a substituent selected from the group consisting of alkyl, alkoxy, hydrogen, nitro, and halogen and R² and R³ are independently selected from the group consisting of alkyl, alkenyl, alkynyl, alkoxy, cycloalkyloxy, amino and phenyl.

In some embodiments, R¹ is phenyl optionally substituted with one or more halogen substituents and R² and R³ are independently selected from the group consisting of alkyl, alkenyl, alkynyl, alkoxy, cycloalkyloxy, amino and phenyl.

In some embodiments, R¹ is optionally substituted phenyl, R⁴ is hydrogen, R⁷ is selected from the group consisting of hydrogen and alkyl, and R⁶ is amino.

In some embodiments, R¹ is unsubstituted phenyl, R⁴ is hydrogen, R⁷ is selected from the group consisting of hydrogen and alkyl, and R⁶ is amino.

In some embodiments, R¹ is phenyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino.

In some embodiments, R¹ is phenyl optionally substituted with one or more halogen substituents, R⁴ is hydrogen, R⁷ is selected from the group consisting of hydrogen and alkyl, and R⁶ is amino.

In some embodiments, R¹ is optionally substituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is unsubstituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is phenyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is phenyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is optionally substituted phenyl and R⁵ is phenyl optionally substituted with one or more substituents selected from the group consisting of halogen, cyano, nitro, and haloalkyl.

In some embodiments, R¹ is unsubstituted phenyl and R⁵ is phenyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen and R⁵ is phenyl optionally substituted with one or more substituents selected from the group consisting of halogen, cyano, nitro, and haloalkyl.

In some embodiments, R¹ is phenyl optionally substituted with one or more halogen substituents and R⁵ is phenyl optionally substituted with one or more substituents selected from the group consisting of halogen, cyano, nitro, and haloalkyl.

In some embodiments, R¹ is optionally substituted phenyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted phenyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen and R² is selected from the group consisting of optionally substituted alkoxy, alkyl and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is phenyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted phenyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is unsubstituted phenyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen, R⁴ is hydrogen, R¹⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is phenyl optionally substituted with one or more halogen substituents, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is optionally substituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is phenyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted phenyl and R⁵ is pyridinyl optionally substituted with one or more substituents selected from the group consisting of halogen, cyano, nitro, and haloalkyl.

In some embodiments, R¹ is unsubstituted phenyl and R⁵ is pyridinyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen and R⁵ is pyridinyl optionally substituted with one or more substituents selected from the group consisting of halogen, cyano, nitro, and haloalkyl.

In some embodiments, R¹ is phenyl optionally substituted with one or more halogen substituents and R⁵ is pyridinyl optionally substituted with one or more substituents selected from the group consisting of halogen, cyano, nitro, and haloalkyl.

In some embodiments, R¹ is optionally substituted phenyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted phenyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is phenyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted phenyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is unsubstituted phenyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is phenyl optionally substituted with one or more halogen substituents, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is optionally substituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted phenyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is phenyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is phenyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted phenyl; R² is alkyl; R³ is alkyl; R⁴ is hydrogen; R⁵ is unsubstituted phenyl; R⁶ is amino; and R⁷ is hydrogen.

In some embodiments, R¹ is unsubstituted phenyl; R² is alkyl; R³ is alkyl; R⁴ is hydrogen; R⁵ is unsubstituted pyridinyl; R⁶ is amino; and R⁷ is hydrogen.

In some embodiments, R¹ is unsubstituted phenyl; R² is alkyl; R³ is alkyl; R⁴ is hydrogen; R⁵ is phenyl substituted with one or more halogen; R⁶ is amino; and R⁷ is hydrogen.

In some embodiments, R¹ is unsubstituted phenyl; R² is alkyl; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is unsubstituted phenyl; R² is alkyl; R³ is alkyl; R⁴ is hydrogen; R⁵ is pyridinyl substituted with one or more halogen; R⁶ is amino; and R⁷ is hydrogen.

In some embodiments, R¹ is unsubstituted phenyl; R² is alkyl; R³ is alkyl; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is unsubstituted phenyl; R² is alkyl; R³ is alkoxy; R⁴ is hydrogen; R⁵ is unsubstituted phenyl; R⁶ is amino; and R⁷ is hydrogen.

In some embodiments, R¹ is unsubstituted phenyl; R² is alkyl; R³ is alkoxy; R⁴ is hydrogen; R⁵ is unsubstituted pyridinyl; R⁶ is amino; and R⁷ is hydrogen.

In some embodiments, R¹ is unsubstituted phenyl; R² is alkyl; R³ is alkoxy; R⁴ is hydrogen; R⁵ is phenyl substituted with one or more halogen; R⁶ is amino; and R⁷ is hydrogen.

In some embodiments, R¹ is unsubstituted phenyl; R² is alkyl; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is unsubstituted phenyl; R² is alkyl; R³ is alkoxy; R⁴ is hydrogen; R⁵ is pyridinyl substituted with one or more halogen; R⁶ is amino; and R⁷ is hydrogen.

In some embodiments, R¹ is unsubstituted phenyl; R² is alkyl; R³ is alkoxy; R⁴ is hydrogen; R⁵ is pyridinyl substituted with one or more substituents selected from the group consisting of cyano, nitro and haloalkyl; R⁶ is amino; and R⁷ is hydrogen.

In some embodiments, R¹ is unsubstituted phenyl; R² is alkoxy; R³ is alkyl; R⁴ is hydrogen; R⁵ is unsubstituted phenyl; R⁶ is amino; and R⁷ is hydrogen.

In some embodiments, R¹ is unsubstituted phenyl; R² is alkoxy; R³ is alkyl; R⁴ is hydrogen; R⁵ is unsubstituted pyridinyl; R⁶ is amino; and R⁷ is hydrogen.

In some embodiments, R¹ is unsubstituted phenyl; R² is alkoxy; R³ is alkyl; R⁴ is hydrogen; R⁵ is phenyl substituted with one or more halogen; R⁶ is amino; and R⁷ is hydrogen.

In some embodiments, R¹ is unsubstituted phenyl; R² is alkoxy; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is unsubstituted phenyl; R² is alkoxy; R³ is alkyl; R⁴ is hydrogen; R⁵ is pyridinyl substituted with one or more halogen; R⁶ is amino; and R⁷ is hydrogen.

In some embodiments, R¹ is unsubstituted phenyl; R² is alkoxy; R³ is alkyl; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is unsubstituted phenyl; R² is alkoxy; R³ is alkoxy; R⁴ is hydrogen; R⁵ is unsubstituted phenyl; R⁶ is amino; and R⁷ is hydrogen.

In some embodiments, R¹ is unsubstituted phenyl; R² is alkoxy; R³ is alkoxy; R⁴ is hydrogen; R⁵ is unsubstituted pyridinyl; R⁶ is amino; and R⁷ is hydrogen.

In some embodiments, R¹ is unsubstituted phenyl; R² is alkoxy; R³ is alkoxy; R⁴ is hydrogen; R⁵ is phenyl substituted with one or more halogen; R⁶ is amino; and R⁷ is hydrogen.

In some embodiments, R¹ is unsubstituted phenyl; R² is alkoxy; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is unsubstituted phenyl; R² is alkoxy; R³ is alkoxy; R⁴ is hydrogen; R⁵ is pyridinyl substituted with one or more halogen; R¹ is amino; and R⁷ is hydrogen.

In some embodiments, R¹ is unsubstituted phenyl; R² is alkoxy; R³ is alkoxy; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is phenyl substituted with one or more halogen substituents; R² is alkyl; R³ is alkyl; R⁴ is hydrogen; R⁵ is unsubstituted phenyl; R⁶ is amino; and R⁷ is hydrogen.

In some embodiments, R¹ is phenyl substituted with one or more halogen substituents; R² is alkyl; R³ is alkyl; R⁴ is hydrogen; R⁵ is unsubstituted pyridinyl; R⁶ is amino; and R⁷ is hydrogen.

In some embodiments, R¹ is phenyl substituted with one or more halogen substituents; R² is alkyl; R³ is alkyl; R⁴ is hydrogen; R⁵ is phenyl substituted with one or more halogen; R⁶ is amino; and R⁷ is hydrogen.

In some embodiments, R¹ is phenyl substituted with one or more halogen substituents; R² is alkyl; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is phenyl substituted with one or more halogen substituents; R² is alkyl; R³ is alkyl; R⁴ is hydrogen; R⁵ is pyridinyl substituted with one or more halogen; R⁶ is amino; and R⁷ is hydrogen.

In some embodiments, R¹ is phenyl substituted with one or more halogen substituents; R² is alkyl; R³ is alkyl; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is unsubstituted phenyl; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is unsubstituted pyridinyl; R⁶ is amino; and R⁷ is hydrogen.

In some embodiments, R¹ is phenyl substituted with one or more halogen substituents; R² is alkyl; R³ is alkoxy; R⁴ is hydrogen; R¹ is phenyl substituted with one or more halogen; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is pyridinyl substituted with one or more halogen; R⁶ is amino; and R⁷ is hydrogen.

In some embodiments, R¹ is phenyl substituted with one or more halogen substituents; R² is alkyl; R³ is alkoxy; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is unsubstituted phenyl; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is unsubstituted pyridinyl; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is phenyl substituted with one or more halogen; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is phenyl substituted with one or more halogen substituents; R² is alkoxy; R³ is alkyl; R⁴ is hydrogen; R⁵ is pyridinyl substituted with one or more halogen; R¹ is amino; and R⁷ is hydrogen.

In some embodiments, R¹ is phenyl substituted with one or more halogen substituents; R² is alkoxy; R³ is alkyl; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is unsubstituted phenyl; R⁶ is amino; and R⁷ is hydrogen,

In some embodiments, R¹ is phenyl substituted with one or more halogen substituents; R² is alkoxy; R³ is alkoxy; R⁴ is hydrogen; R⁵ is unsubstituted pyridinyl; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is phenyl substituted with one or more halogen; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is pyridinyl substituted with one or more halogen; R⁶ is amino; and R⁷ is hydrogen.

In some embodiments, R¹ is phenyl substituted with one or more halogen substituents; R² is alkoxy; R³ is alkoxy; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is pyridinyl substituted with one or more halogen substituents; R² is alkyl; R³ is alkyl; R⁴ is hydrogen; R⁵ is unsubstituted phenyl; R⁶ is ammo; and R⁷ is hydrogen.

In some embodiments, R¹ is pyridinyl substituted with one or more halogen substituents; R² is alkyl; R³ is alkyl; R⁴ is hydrogen; R¹ is unsubstituted pyridinyl; R⁶ is amino; and R⁷ is hydrogen.

In some embodiments, R¹ is pyridinyl substituted with one or more halogen substituents; R² is alkyl; R³ is alkyl; R⁴ is hydrogen; R⁵ is phenyl substituted with one or more halogen; R⁶ is amino; and R⁷ is hydrogen.

In some embodiments, R¹ is pyridinyl substituted with one or more halogen substituents; R² is alkyl; R³ is alkyl; R⁴ is hydrogen; R⁵ is phenyl substituted with one or more cyano, nitro or haloacyl substituents; R⁶ is amino; and R⁷ is hydrogen.

In some embodiments, R¹ is optionally substituted monocyclic heterocyclyl.

In some embodiments, R¹ is unsubstituted monocyclic heterocyclyl.

In some embodiments, R¹ is monocyclic heterocyclyl optionally substituted with one or more substituents selected from the group consisting of alkyl, alkoxy, hydrogen, nitro, and halogen.

In some embodiments, R¹ is monocyclic heterocyclyl optionally substituted with one or more halogen substituents.

In some embodiments, R¹ is optionally substituted monocyclic heterocyclyl, R⁴ is hydrogen, R⁷ is selected from the group consisting of hydrogen and alkyl, and R⁶ is amino.

In some embodiments, R¹ is unsubstituted monocyclic heterocyclyl, R⁴ is hydrogen, R⁷ is selected from the group consisting of hydrogen and alkyl, and R⁶ is amino.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino.

In some embodiments, R¹ is optionally substituted monocyclic heterocyclyl and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is unsubstituted monocyclic heterocyclyl and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is monocyclic heterocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is monocyclic heterocyclyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is optionally substituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is unsubstituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyd and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is optionally substituted monocyclic heterocyclyl and R⁵ is phenyl optionally substituted with one or more substituents selected from the group consisting of halogen, cyano, nitro, and haloalkyl.

In some embodiments, R¹ is unsubstituted monocyclic heterocyclyl and R⁵ is phenyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen and R⁵ is phenyl optionally substituted with one or more substituents selected from the group consisting of halogen, cyano, nitro, and haloalkyl.

In some embodiments, R¹ is monocyclic heterocyclyl optionally substituted with one or more halogen substituents and R⁵ is phenyl optionally substituted with one or more substituents selected from the group consisting of halogen, cyano, nitro, and haloalkyl.

In some embodiments, R¹ is optionally substituted monocyclic heterocyclyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is unsubstituted monocyclic heterocyclyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is optionally substituted monocyclic heterocyclyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted monocyclic heterocyclyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic heterocyclyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted monocyclic heterocyclyl and R⁵ is pyridinyl optionally substituted with one or more substituents selected from the group consisting of halogen, cyano, nitro, and haloalkyl.

In some embodiments, R¹ is unsubstituted monocyclic heterocyclyl and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more halogen substituents and R⁵ is pyridinyl optionally substituted with one or more substituents selected from the group consisting of halogen, cyano, nitro, and haloalkyl.

In some embodiments, R¹ is optionally substituted monocyclic heterocyclyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is unsubstituted monocyclic heterocyclyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is optionally substituted monocyclic heterocyclyl and R² is selected from the group consisting of optionally substituted alkoxy, alkyl, and optionally substituted amino and R¹ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted monocyclic heterocyclyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen and R³ is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and phenyl and R⁵ is pyridinyl optionally substituted with one or more substituents selected from the group consisting of halogen, cyano, nitro, and haloalkyl.

In some embodiments, R¹ is unsubstituted monocyclic heterocyclyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more substituents selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is monocyclic heterocyclyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted pyridinyl.

In some embodiments, R¹ is unsubstituted pyridinyl.

In some embodiments, R¹ is pyridinyl optionally substituted with a substituent selected from the group consisting of alkyl, alkoxy, hydrogen, nitro, and halogen.

In some embodiments, R¹ is pyridinyl optionally substituted with one or more halogen substituents.

In some embodiments, R¹ is optionally substituted pyridinyl and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is unsubstituted pyridinyl and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is pyridinyl optionally substituted with a substituent selected from the group consisting of alkyl, alkoxy, hydrogen, nitro, and halogen and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is pyridinyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is optionally substituted pyridinyl, R⁴ is hydrogen, R⁷ is selected from the group consisting of hydrogen and alkyl, and R⁶ is amino.

In some embodiments, R¹ is unsubstituted pyridinyl, R⁴ is hydrogen, R⁷ is selected from the group consisting of hydrogen and alkyl, and R⁶ is amino.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino.

In some embodiments, R¹ is pyridinyl optionally substituted with one or more halogen substituents, R⁴ is hydrogen, R⁷ is selected from the group consisting of hydrogen and alkyl, and R⁶ is amino. In some embodiments, R¹ is optionally substituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is unsubstituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl.

In some embodiments, R¹ is pyridinyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R¹ is selected from the group consisting of C₂-C₈-alkyl, alkoxy, phenyl, and cycloalkyl.

In some embodiments, R¹ is optionally substituted pyridinyl and R⁵ is phenyl optionally substituted with one or more substituents selected from the group consisting of halogen, cyano, nitro, and haloalkyl.

In some embodiments, R¹ is unsubstituted pyridinyl and R⁵ is phenyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen and R⁵ is phenyl optionally substituted with one or more substituents selected from the group consisting of halogen, cyano, nitro, and haloalkyl.

In some embodiments, R¹ is pyridinyl optionally substituted with one or more halogen substituents and R⁵ is phenyl optionally substituted with one or more substituents selected from the group consisting of halogen, cyano, nitro, and haloalkyl.

In some embodiments, R¹ is optionally substituted pyridinyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted pyridinyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is pyridinyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted pyridinyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is unsubstituted pyridinyl, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and all, and R⁶ is amino and R⁵ is phenyl optionally substituted with one or more substituents selected from the group consisting of halogen, cyano, nitro, and haloalkyl.

In some embodiments, R¹ is pyridinyl optionally substituted with one or more halogen substituents, R⁴ is hydrogen, R⁷ is selected from the group consisting of hydrogen and alkyl, and R⁶ is amino and R⁵ is phenyl optionally substituted with one or more substituents selected from the group consisting of halogen, cyano, nitro, and haloalkyl.

In some embodiments, R¹ is optionally substituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is unsubstituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is pyridinyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is phenyl optionally substituted with one or more substituents selected from the group consisting of halogen, cyano, nitro, and haloalkyl.

In some embodiments, R¹ is optionally substituted pyridinyl and R⁵ is pyridinyl optionally substituted with one or more substituents selected from the group consisting of halogen, cyano, nitro, and haloalkyl.

In some embodiments, R¹ is unsubstituted pyridinyl and R⁵ is pyridinyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen and R⁵ is pyridinyl optionally substituted with one or more substituents selected from the group consisting of halogen, cyano, nitro, and haloalkyl.

In some embodiments, R¹ is pyridinyl optionally substituted with one or more halogen substituents and R⁵ is pyridinyl optionally substituted with one or more substituents selected from the group consisting of halogen, cyano, nitro, and haloalkyl.

In some embodiments, R¹ is optionally substituted pyridinyl and R⁵ is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted pyridinyl and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is pyridinyl optionally substituted with one or more halogen substituents and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is optionally substituted pyridinyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

in some embodiments, R¹ is unsubstituted pyridinyl, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is pyridinyl optionally substituted with one or more halogen substituents, R⁴ is hydrogen, R⁷ is selected from the group consisting of hydrogen and alkyl, and R⁶ is amino and R⁵ is pyridinyl optionally substituted with one or more substituents selected from the group consisting of halogen, cyano, nitro, and haloalkyl.

In some embodiments, R¹ is optionally substituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted pyridinyl, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is pyridinyl optionally substituted with a substituent selected from the group consisting of alkyl, alkoxy, hydrogen, nitro, and halogen, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is pyridinyl optionally substituted with one or more halogen substituents, R⁴ is hydrogen, R⁷ is selected from the group consisting of hydrogen and alkyl, and R⁶ is amino and R² is selected from the group consisting of optionally substituted alkoxy, alkyl, and optionally substituted amino and R³ is selected from the group consisting of C₂-C₈-alkyl, alkoxy, phenyl, and cycloalkyl and R⁵ is pyridinyl optionally substituted with one or more substituents selected from the group consisting of halogen, cyano, nitro, and haloalkyl.

In some embodiments, R¹ is unsubstituted pyridinyl; R² is alkyl; R³ is alkyl; R⁴ is hydrogen; R⁵ is unsubstituted phenyl; R⁶ is amino; and R⁷ is hydrogen.

In some embodiments, R¹ is unsubstituted pyridinyl; R² is alkyl; R³ is alkyl; R⁴ is hydrogen; R⁵ is pyridinyl R⁶ is amino; and R⁷ is hydrogen.

In some embodiments, R¹ is unsubstituted pyridinyl; R² is alkyl; R³ is alkyl; R⁴ is hydrogen; R⁵ is phenyl substituted with one or more halogen; R⁶ is amino; and R⁷ is hydrogen.

In some embodiments, R¹ is unsubstituted pyridinyl; R² is alkyl; R³ is alkyl; R⁴ is hydrogen; R⁵ is phenyl substituted with one or more cyano, nitro or haloalkyl substituents; R¹ is amino; and R⁷ is hydrogen.

In some embodiments, R¹ is unsubstituted pyridinyl; R² is alkyl; R³ is alkyl; R⁴ is hydrogen; R⁵ is pyridinyl substituted with one or more halogen; R⁶ is amino; and R⁷ is hydrogen.

In some embodiments, R¹ is unsubstituted pyridinyl; R² is alkyl; R³ is alkyl; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is unsubstituted pyridinyl; R² is alkyl; R³ is alkoxy; R⁴ is hydrogen; R⁵ is unsubstituted phenyl; R⁶ is amino; and R⁷ is hydrogen.

In some embodiments, R¹ is unsubstituted pyridinyl; R² is alkyl; R³ is alkoxy; R⁴ is hydrogen; R⁵ is unsubstituted pyridinyl; R⁶ is amino; and R⁷ is hydrogen.

In some embodiments, R¹ is unsubstituted pyridinyl; R² is alkyl; R³ is alkoxy; R⁴ is hydrogen; R⁵ is phenyl substituted with one or more halogen; R⁶ is amino; and R⁷ is hydrogen.

In some embodiments, R¹ is unsubstituted pyridinyl; R² is alkyl; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is unsubstituted pyridinyl; R² is alkyl; R³ is alkoxy; R⁴ is hydrogen; R⁵ is pyridinyl substituted with one or more halogen; R⁶ is amino; and R⁷ is hydrogen.

In some embodiments, R¹ is unsubstituted pyridinyl; R² is alkyl; R³ is alkoxy; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is unsubstituted pyridinyl; R² is alkoxy; R³ is alkyl; R⁴ is hydrogen; R⁵ is unsubstituted phenyl; R⁶ is amino; and R⁷ is hydrogen.

In some embodiments, R¹ is unsubstituted pyridinyl; R² is alkoxy; R³ is alkyl; R⁴ is hydrogen; R¹ is unsubstituted pyridinyl; R⁶ is amino; and R⁷ is hydrogen.

In some embodiments, R¹ is unsubstituted pyridinyl; R² is alkoxy; R³ is alkyl; R⁴ is hydrogen; R⁵ is phenyl substituted with one or more halogen; R⁶ is amino; and R⁷ is hydrogen.

In some embodiments, R¹ is unsubstituted pyridinyl; R² is alkoxy; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is unsubstituted pyridinyl; R² is alkoxy; R³ is alkyl; R⁴ is hydrogen; R⁵ is pyridinyl substituted with one or more halogen; R⁶ is amino; and R⁷ is hydrogen.

In some embodiments, R¹ is unsubstituted pyridinyl; R² is alkoxy; R³ is alkyl; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is unsubstituted pyridinyl; R² is alkoxy; R³ is alkoxy; R⁴ is hydrogen; R⁵ is unsubstituted phenyl; R⁶ is amino; and R⁷ is hydrogen.

In some embodiments, R¹ is unsubstituted pyridinyl; R² is alkoxy; R³ is alkoxy; R⁴ is hydrogen; R⁵ is unsubstituted pyridinyl; R⁶ is amino; and R⁷ is hydrogen.

In some embodiments, R¹ is unsubstituted pyridinyl; R² is alkoxy; R³ is alkoxy; R⁴ is hydrogen; R⁵ is phenyl substituted with one or more halogen; R⁶ is amino; and R⁷ is hydrogen.

In some embodiments, R¹ is unsubstituted pyridinyl; R² is alkoxy; R³ is alkoxy; R⁴ is hydrogen; R⁵ is phenyl substituted with one or more cyano, nitro or haloalkyl substituents; M is amino; and R⁷ is hydrogen.

In some embodiments, R¹ is unsubstituted pyridinyl; R² is alkoxy; R³ is alkoxy; R⁴ is hydrogen; R⁵ is pyridinyl substituted with one or more halogen; R⁶ is amino; and R⁷ is hydrogen.

In some embodiments, R¹ is unsubstituted pyridinyl; R² is alkoxy; R³ is alkoxy; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is pyridinyl substituted with one or more halogen substituents; R² is alkyl; R³ is alkyl; R⁴ is hydrogen; R⁵ is pyridinyl substituted with one or more halogen; R⁶ is amino; and R⁷ is hydrogen.

In some embodiments, R¹ is pyridinyl substituted with one or more halogen substituents; R² is alkyl; is alkyl; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is unsubstituted phenyl; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is unsubstituted pyridinyl; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is phenyl substituted with one or more halogen; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is phenyl substituted with one or more cyano, nitro or haloalkyl substituents; R⁶ is amino; and R⁷ is hydrogen.

In some embodiments, R¹ is pyridinyl substituted with one or more halogen substituents; R² is alkyl; R³ is alkoxy; R⁴ is hydrogen; R⁵ is pyridinyl substituted with one or more halogen; R⁶ is amino; and R⁷ is hydrogen.

In some embodiments, R¹ is pyridinyl substituted with one or more halogen substituents; R¹ is alkyl; R³ is alkoxy; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is pyridinyl substituted with one or more halogen substituents; R² is alkoxy;

R³ is alkyl; R⁴ is hydrogen; R⁵ is unsubstituted phenyl; R⁶ is amino; and R⁷ is hydrogen.

In some embodiments, R¹ is pyridinyl substituted with one or more halogen substituents; R² is alkoxy; R³ is alkyl; R⁴ is hydrogen; R⁵ is unsubstituted pyridinyl; R⁶ is amino; and R⁷ is hydrogen.

In some embodiments, R¹ is pyridinyl substituted with one or more halogen substituents; R² is alkoxy; R³ is alkyl; R⁴ is hydrogen; R⁵ is phenyl substituted with one or more halogen; R⁶ is amino; and R⁷ is hydrogen.

In some embodiments, R¹ is pyridinyl substituted with one or more halogen substituents; R² is alkoxy; R³ is alkyl; R⁴ is hydrogen; R⁵ is phenyl substituted with one or more cyano, nitro or haloalkyl substituents; R⁶ is amino; and R⁷ is hydrogen.

In some embodiments, R¹ is pyridinyl substituted with one or more halogen substituents; R² is alkoxy; R³ is alkyl; R⁴ is hydrogen; R⁵ is pyridinyl substituted with one or more halogen; R⁶ is amino; and R⁷ is hydrogen.

In some embodiments, R¹ is pyridinyl substituted with one or more halogen substituents; R² is alkoxy; R³ is alkyl; R⁴ is hydrogen; R⁵ is pyridinyl substituted with one or more substiuents selected from the group consisting of cyano, nitro and haloalkyl; R⁶ is amino; and R⁷ is hydrogen.

In some embodiments, R¹ is pyridinyl substituted with one or more halogen substituents; R² is alkoxy; R³ is alkoxy; R⁴ is hydrogen; R⁵ is unsubstituted phenyl; R⁶ is amino; and R⁷ is hydrogen.

In some embodiments, R¹ is pyridinyl substituted with one or more halogen substituents; R² is alkoxy; R³ is alkoxy; R⁴ is hydrogen; R⁵ is unsubstituted pyridinyl; R⁶ is amino; and R⁷ is hydrogen.

In some embodiments, R³ is pyridinyl substituted with one or more halogen substituents; R² is alkoxy; R³ is alkoxy; R⁴ is hydrogen; R⁵ is phenyl substituted with one or more halogen; R⁶ is amino; and R⁷ is hydrogen.

In some embodiments, R¹ is pyridinyl substituted with one or more halogen substituents; R¹ is alkoxy; R³ is alkoxy; R⁴ is hydrogen; R¹ is phenyl substituted with one or more cyano, nitro or haloalkyl substituents; R⁶ is amino; and R⁷ is hydrogen.

In some embodiments, R¹ is pyridinyl substituted with one or more halogen substituents; R² is alkoxy; R³ is alkoxy; R⁴ is hydrogen; R⁵ is pyridinyl substituted with one or more halogen; R⁶ is amino; and R⁷ is hydrogen.

In some embodiments, R¹ is pyridinyl substituted with one or more halogen substituents; R² is alkoxy; R³ is alkoxy; R⁴ is hydrogen; R⁵ is pyridinyl substituted with one or more substiuents selected from the group consisting of cyano, nitro and haloalkyl; R⁶ is amino; and R⁷ is hydrogen.

In the present invention, 4,7-dihydrothieno[2,3-b]pyridine compounds (and salts thereof) and pharmaceutical compositions are administered in an amount and through a route sufficient to achieve an upregulation of the alpha-myosin heavy chain (alpha-MyHC) or sarco endoplasmic reticulum Ca²⁺ ATPase (SERCA) mRNA or protein levels.

In some embodiment, a method is provided for increasing the concentration of the alpha-myosin heavy chain (MyHC) mRNA or protein levels, the method comprising administering to a subject a compound (or salt thereof) of the invention.

In some embodiments, compounds (or salts thereof) of the invention are useful for increasing the concentration of the alpha-myosin heavy chain (MyHC) mRNA levels. In other embodiments, compounds (or salts thereof) of the invention are useful for increasing the concentration of the alpha-myosin heavy chain (MyHC) protein levels.

In some embodiments, the alpha-MyHC concentration is increased in a human with a cardiovascular condition. In other embodiments, the cardiovascular condition includes pathological hypertrophy, chronic heart failure and/or acute heart failure. In yet other embodiments, the cardiovascular condition includes dilated cardiomyopathy, coronary artery disease, myocardial infarction, congestive heart failure and/or cardiac hypertrophy. In further embodiments, the cardiovascular condition is myogcardial infarction. In yet further embodiments, the cardiovascular condition is cardiac hypertrophy

It is contemplated that the formulations of the current invention will be adininistered to a cell, that cell being an intact cardiomyocyte. These cardiomyocytes are located in heart tissue and that heart may be the intact heart of a human patient. It is further contemplated that the formulations may be administered directly to the ventricle, and specifically the left ventricle of the heart. Routes include intra-arterial, intravenous, intramuscular and oral routes.

In some embodiments, the alpha-MyHC is upregulated in cardiomyocytes. In further embodiments, the compounds (or salts thereof) of the invention are administered in an amount and route sufficient to achieve an increase in the contractility of cardiomyoctes.

In another embodiment, a method is provided for treating cardiovascular condition in a patient comprising administration of compounds (or salts thereof) of the invention, wherein the compound is other than,

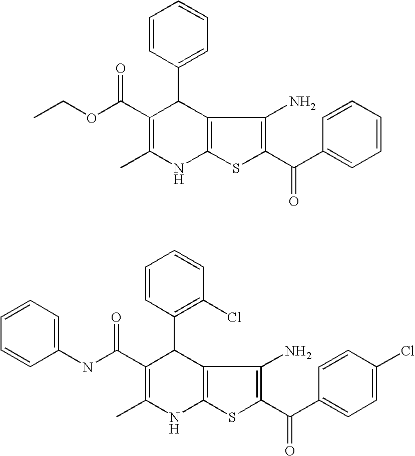

in an amount and in a route sufficient to treat cardiovascular disease. The term “treating” as used in this patent application means ameliorating, suppressing, eradicating, preventing, reducing the risk of and/or delaying the onset of the condition being treated.

In one embodiment of the present invention, methods for the treatment of cardiac hypertrophy or heart failure using compounds (or salts thereof) of the invention are provided. Here, treatment comprises reducing one or more of the symptoms of cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness (the same results may hold true for the right ventricle). In addition, use of the present invention may prevent cardiac hypertrophy and its associated symptoms from arising.

Heart failure, which encompasses a wide array of cardiomyopathies, is one of the leading causes of morbidity and mortality in the world. In the U.S. alone, estimates indicate that 3 million people are currently living with one form of cardiomyopathy, and another 500,000 are diagnosed on a yearly basis. Dilated cardiomyopathy (DCM), a specific form of heart failure, also referred to as “congestive cardiomyopathy,” is the most common form of the cardiomyopathies and has an estimated prevalence of nearly 40 per 100,000 individuals (Durand et al., 1995). Although there are other causes of DCM, familial dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin). In addition, many DCM patients are chronic alcoholics. Fortunately, for these patients, the progression of myocardial dysfunction may be stopped or reversed if alcohol consumption is reduced or stopped early in the course of disease. Peripartum cardiomyopathy is another idiopathic form of DCM, as is disease associated with infectious sequelac. Collectively, cardiomyopathies, including DCM, are significant public health problems.

Heart disease and its manifestations, including coronary artery disease, myocardial infarction, congestive heart failure and cardiac hypertrophy, clearly presents a major health risk in the United States today. The cost to diagnose, treat and support patients suffering from these diseases is well into the billions of dollars. Two particularly severe manifestations of heart disease are myocardial infarction and cardiac hypertophy.

With respect to myocardial infarction, typically an acute thrombocytic coronary occlusion occurs in a coronary artery as a result of atherosclerosis and causes myocardial cell death. Because cardiomyocytes are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic.

With respect to cardiac hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been fully elucidated. Understanding these mechanisms is a major concern in the prevention and treatment of cardiac disease and will be crucial as a therapeutic modality in designing new drugs that specifically target cardiac hypertrophy and cardiac heart failure. As pathologic cardiac hypertrophy typically does not produce any symptoms until the cardiac damage is severe enough to produce bean failure, the symptoms of cardiomyopathy are those associated with heart failure. These symptoms include shortness of breath, fatigue with exertion, e inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Diagnosis of dilated cardiomyopathy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

As mentioned above, treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

Thus, the currently used pharmacological agents have severe shortcomings in particular patient populations. The availability of new, safe and effective agents would undoubtedly benefit patients who either cannot use the pharmacological modalities presently available, or who do not receive adequate relief from those modalities. The prognosis for patients with DCM is variable, and depends upon the degree of ventricular dysfunction, with the majority of deaths occurring within five years of diagnosis.

Current medical management of cardiac hypertrophy in the setting of a cardiovascular disorder includes the use of at least two types of drugs: inhibitors of the renin-angiotensin system, and β-adrenergic blocking agents (Bristow, 1999). Therapeutic agents to treat pathologic hypertrophy in the setting of heart failure include angiotensin II converting enzyme (ACE) inhibitors and β-adrenergic receptor blocking agents (Eichhorn and Bristow, 1996). Other pharmaceutical agents that have been disclosed for treatment of cardiac hypertrophy include but are not limited to angiotensin II receptor antagonists (U.S. Pat. No. 5,604,251) and neuropeptide γ antagonists (WO 98/33791). Despite currently available pharmaceutical compounds, prevention and treatment of cardiac hypertrophy, and subsequent heart failure, continues to present a major therapeutic challenge.

Another potential therapeutic approach is to reverse the structural changes that occur in the heart in response to hypertrophy and heart failure, a process known as cardiac remodeling. Remodeling relates specifically to the gene expression changes that occur as the heart grows more diseased. In remodeling, genes normally expressed during fetal development (fetal genes such as SE-RCA, alpha-MyHC, etc.) are expressed aberrantly (for a review see Lowes et al, 2002, hereinafter incorporated by reference). Originally these changes were thought to be irreversible, so the only hope was to provide therapy to alleviate the symptoms. However, it was eventually discovered that unloading the failing human heart by placing the patient on a left ventricular assist device could reverse some of the remodeling changes (Dipla et al, 1998). Recently it has been demonstrated that this reverse remodeling can occur through pharmaceutical therapies. (Bristow et al., 2000). Through the use of acetylcholine-esterase inhibitors, improvements in cardiac contractility have been seen and systolic function of the heart has been enhanced. (Eichorn et al., 1996; Lowes et al., 2002). Furthermore, beta-adrenergic receptor blockers have been shown to upregulate mRNA levels of alpha-MyHC and SERCA through indirect action on other cardiac targets (Lowes et at, 2002). It is therefore plausible that treating the underlying contractile defects in the remodeled heart by directly upregulating alpha-MyHC would lead to a reversal of the remodeling process.

In another embodiment, a method is provided for inducing a reversal of remodeling in hypertrophic and failing heart tissue in viva, wherein the method comprises administration of a substantially pure compound (or salt thereof) of the invention.

In a further embodiment, there is disclosed a method of inducing a reversal of the remodeling that occurs in hypertrophic or failing heart tissue in vivo, comprising administering to a subject suffering from cardiac hypertrophy or heart failure an amount of the claimed formulation that is sufficient to induce reverse remodeling, remodeling being defined as a decrease in the expression of the fetal genes and an increase in the expression of normal cardiac genes.

Thyroid hormones are essential for normal growth and differentiation in mammals, and play a critical role on maintaining metabolic homeostasis. For example, thyroid hormones participate in the regulation of the metabolism of lipids, sugars, proteins and energies. Thyroid hormones also affect cardiovascular function such as heart rate, cardiac contraction, peripheral vascular resistance and the like.

A naturally occurring hormone, 3,5,3′-triiodo-L-thyronine (hereinafter referred to as T3) binds to nuclear THR. A complex composed of T3 and THR binds to the promoter region of T3 regulatory genes, which is referred to as thyroid hormone response element (TRE), located at the upstream of target genes, and activates or suppresses the expression of these genes. Thyroid hormones exhibit the majority of actions by regulating the expression of the target genes in nucleus.

Patients with hypothyroid disorders may manifest symptoms such as decreased body temperature, increased body weight, increased serum cholesterol, decreased cardiac functions, liver function disorders, depression, dry skins or alopecia. In contrast, increased body temperature, decreased body weight, decreased serum cholesterol, tachycardia, increased stroke volume, arrhythmia or increased bone absorption are observed in patients with hyperthyroidism. As discussed above, thyroid hormones participate in the regulation of various physiological actions in vivo, and ligands having an affinity to thyroid hormone receptors have been expected to be useful as a therapeutic agent for hyperlipidemia, atherosclerosis, obesity, diabetes mellitus, arrhythmia, congestive heart failure, hypertension, depression, osteoporosis, glaucoma, skin disorders, alopecia and the like (for a full review of the uses of thyroid replacement hormone, see European Patent Application 1,471,049, hereby fully incorporated by reference). It has been reported that the administration of a thyroid hormone ameliorated fatty liver and decreased the amount of liver fiber (Huang et al., 2001; Yasna et al., 1993). It has also been demonstrated that the administration of a thyroid hormone decreased the amount of liver glutathione, and in a rat hepatocarcinogenesis model, decreased the incidence of liver cancer and suppressed metastases to lung (Huang et al., 2001; Yasna et al., 1993). Accordingly, thyroid hormone receptor ligands are expected to be useful for the treatment of fatty liver, liver cirrhosis and liver cancer.

Thyroid hormones are currently used primarily as replacement therapy for patients with hypothyroidism. Further attempts to use thyroid hormones in the treatment of hyperlipidemia, obesity, depression or skin disorders have been made. However, it is reported that administering thyroid hormones at dosages more than those of replacement therapy is often accompanied with cardiac toxicities such as arrhythmia, angina, cardiac failure and the like. The inventors herein have described a novel class of compounds capable of being used as thyroid hormone replacement.

In other embodiments of the invention, compounds (or salts thereof) of the invention and pharmaceutical formulations can be used in a method for treating a disease state in a patient where modulation of the thyroid hormone receptor is beneficial. Such disease states may include but are not limited to one or more of atherosclerosis, syndrome X, metabolic syndrome, familiar hypercholesterolemia, lipid disorders, arterial patency, obesity, weight disorders, hypertension (or hypertension induced by weight gain), exercise intolerance, hypothyroidism, and hyperthyroidism. Specifically, in certain embodiments, compounds (or salts thereof) of the present invention are useful for the treatment of or prevention of atherosclerosis, and in yet further embodiments these compounds (or salts thereof) may be used to prevent or reverse the buildup of arterial plaques or reduce the level of undesirable lipids including cholesterol or its conjugates in patients in need of such therapy. The compounds (or salts thereof) may also be used to prevent abnormal weight gain or weight loss, often associated with hyper or hypothyroidism. In certain embodiments it is contemplated that compounds (or salts thereof) of the current invention can be used to increase the basal metabolic rate of a patient.

Lipid is the scientific term for fats in the blood, and the term is used to describe fatty acids, neutral fats, waxes, and steroids. The two main types of lipids that affect heart disease are fatty acids and cholesterol. Three fatty acid molecules combined with glycerol is known as a triglyceride; when triglycerides are mixed with cholesterol, they can form cholesterol-esters, and combining cholesterol or its esters with phosphorus makes phospbolipids.

During aging, coronary arteries can harden, which leads to athersclerosis, also defined as the buildup of fatty streaks and cholesterol-laden plaque in the artery walls. Coronary heart disease is diagnosed when the accumnulation of plaque in a coronary artery grows large enough to obstruct blood flow to the heart.

Because lipids are hydrophobic and do not readily dissolve in aqueous solution, cholesterol and fatty acids need to be carried in the blood by apoproteins to transport the lipids through the blood and into the cells. These protein-bound fats are called lipoproteins, and lipid disorders generally means problems with the amounts of these lipoproteins in the blood.

Each lipoprotein contains cholesterol, cholesterol-esters, triglycerides, phospholipids, vitamins, and apoproteins. Lipoproteins are classified based on their density, from high density lipoproteins (HDL, which is also called the good cholesterol, and which removes excess cholesterol in the blood and the body by carrying it back to the liver to be broken down), to low density lipoproteins (LDL, also called the bad cholesterol, which also deposits cholesterol in body tissues to be used for cell repair or for energy high levels of LDL), to very low density lipoproteins (VLDL, which is made up mostly of a core of triglycerides, small amounts of proteins, and cholesterol).

Some known lipid disorders include: Primary elevated cholesterol (LDL levels of more than 130 milligrams per deciliter, or mg/dL); dyslipidemic syndrome (also called syndrome x, a group of metabolic risk factors that significantly increases the risk of developing CHD); primary elevated triglycerides (triglyceride level as high as 1500 mg/dL); primary low-HDL syndromes (also called dyslipidemia or dyslipoproteinemia), in which HDL is less than 35 mg/dL; hyperlipidemia, or high cholesterol; familial hypercholesterolemia, (a genetic disorder that increases total and LDL cholesterol); and familial hypertriglyceridemia, inherited high triglycerides. Some lipid disorders are caused by additional or pre-existing diseases or medical conditions, and are called secondary lipid disorders. Secondary lipid disorders may be associated with diabetes mellitus, hypothyroidism, obstructive liver disease, kidney failure, and even steroid use. Current methods of treatment include statins, bile acid sequestrants, fibrates, and niacin (Vitamin B3). The current treatments for lipid disorders vary not only in style of treatment but efficacy and tolerability, but a large unmet need still exists for a viable and well tolerated treatment.

In some embodiments, compounds (or salts thereof) of the invention may provide suitable therapeutic treatment for certain lipid disorders or lipid diseases

In some embodiments, the compounds (or salts thereof) of the invention may lower lipid levels or may lower LDL levels or may lower cholesterol levels or may elevate HDL levels.

In all of the above embodiments, treatment regimens would vary depending on the clinical situation. However, long term maintenance would appear to be appropriate in most circumstances.

The formulations of the current invention may also be combined with, added to, or mixed with additional pharmaceutical formulations or treatment regimens given to the patient or to the heart or to the cardiomyocytes. These additional formulations may include, but are not limited to, “beta blockers,” anti-hypertensives, cardiotonics, antithroibotics, vasodilators, hormone antagonists, endothelin antagonists, cytokine inhibitors and/or blockers, calcium channel blockers, phosphodiesterase inhibitors, and angiotensin type-2 antagonists. These drugs may be given before, at the same time as, or after the compounds (or salts thereof) of the present invention.

In another embodiment, it is envisioned to use the present invention in combination with other therapeutic modalities. Thus, in addition to the therapies described above, one may also provide to the patient more “standard” pharmaceutical cardiac therapies. Examples of other therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by contacting cardiac cells with a single composition or pharmacological formulation that includes both agents, or by contacting the cell with two distinct compositions or formulations, at the same time. Alternatively, the therapy using the claimed formulations may precede or follow administration of the other agent(s) by intervals ranging from minutes to weeks. In embodiments where the various agents are applied separately to the cell, one would generally ensure that a significant period of time did not expire between the time of each delivery, such that the agents would still be able to exert an advantageously combined effect on the cell. In such instances, it is contemplated that one would typically contact the cell with both modalities within about 12-24 hrs of each other and, more preferably, within about 6-12 hrs of each other, with a delay time of only about 12 hrs being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the claimed compounds (or salts thereof, or the other agent will be desired. In this regard, various combinations may be employed. By way of illustration, where the present invention is “A” and the other agent is “B,” the following permutations based on 3 and 4 total administrations are exemplary:

| | | | | | |
|---|---|---|---|---|---|
| A/B/A | B/A/B | B/B/A | A/A/B | B/A/A | A/B/B |
| B/B/B/A | B/B/A/B | A/A/B/B | A/B/A/B | A/B/B/A | B/B/A/A |
| B/A/B/A | B/A/A/B | B/B/B/A | A/A/A/B | B/A/A/A | A/B/A/A |
| A/A/B/A | A/B/B/B | B/A/B/B | B/B/A/B | | |

Other combinations are likewise contemplated. Pharmacological therapeutic agents and methods of administration, dosages, etc., are well known to those of skill in the art (see for example, the “Physicians Desk Reference,” Goodman & Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof. In addition, it should be noted that any of the following may be used to develop new sets of cardiac therapy target genes. While it is expected that many of these genes may overlap, new gene targets likely can be developed.

It will be understood that in the discussion of formulations and methods of treatment, references to the compounds or salts thereof of the present invention are meant to also include the pharmaceutically acceptable salts, solvates, hydrates and polymorphs as well as pharmaceutical compositions comprising these compounds. Also provided are treatments of cardiovascular disease, comprising administering to a subject an effective amount of a compound (or salt thereof) of the present invention and their pharmaceutically acceptable salts, solvates, hydrates and polymorphs and a pharmaceutically acceptable carrier or formulation.

In specific embodiments of the invention the pharmaceutical formulation will be formulated for delivery via rapid release, other embodiments contemplated include but are not limited to timed release, delayed release, and sustained release. The formulation can be an oral suspension or solution in either the solid or liquid form. In father embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl alcohol, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol, Sweetening agents such as those set forth above, and flavoring agents may be added to provide a palatable oral preparation. These compositions may be preserved by the addition of an anti-oxidant such as ascorbic acid,

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds (or salts thereof) of the present invention may also be administered in the form of suppositories for rectal administration of the drug. These compositions can be prepared by mixing the drug with a suitable non-irritating excipient which is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing the compound (or salt thereof) of present invention are employed. For purposes of this application, topical application shall include mouthwashes and gargles.

The formulation may also be administered as nanoparticles, liposomes, granules, inhalants, nasal solutions, or intravenous admixtures.

The previously mentioned formulations are all contemplated for treating patients suffering from cardiovascular disease. Cardiovascular disease includes but is not limited to pathological hypertrophy, chronic and acute heart failure.

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

The term “alkyl” (alone or in combination with another term(s)) means a straight- or branched-Chain saturated hydrocarbyl typically containing from 1 to about 20 carbon atoms, more typically from 1 to about 8 carbon atoms, and even more typically from 1 to about 6 carbon atoms. Examples of such substituents include methyl (Me), ethyl (Et), n-propyl (Pr), isopropyl (iPr), n-butyl (Bu), isobutyl (iBu), sec-butyl, tert-butyl, pentyl, iso-amyl, hexyl, octyl, and the like.

The term “alkenyl” (alone or in combination with another term(s)) means a straight- or branched-Chain hydrocarbyl containing one or more double bonds and typically from 1 to about 20 carbon atoms, more typically from 2 to about 20 carbon atoms, still more typically from about 2 to about 8 carbon atoms, and even more typically from about 2 to about 6 carbon atoms. Examples of such substituents include ═CH₂; ethenyl (vinyl); 2-propenyl; 3-propenyl; 1,4-pentadienyl; 1,4-butadienyl; 1-butenyl; 2-butenyl; 3-butenyl; decenyl; and the like.

The term “alkynyl” (alone or in combination with another term(s)) means a straight- or branched-Chain hydrocarbyl containing one or more triple bonds and typically from 2 to about 20 carbon atoms, more typically from about 2 to about 8 carbon atoms, and even more typically from about 2 to about 6 carbon atoms. Examples of such substituents include ethynyl, 2-propynyl, 3-propynyl, decynyl, 1-butynyl, 2-butynyl, 3-butynyl, and the like.

The term “carbocyclyl” (alone or in combination with another term(s)) means a saturated cyclic (i.e., “cycloalkyl”), partially saturated cyclic, or aryl hydrocarbyl containing from 3 to 14 carbon ring atoms (“ring atoms” are the atoms bound together to form the ring or rings of a cyclic group). A carbocyclyl may be a single ring, which typically contains from 3 to 6 ring atoms. Examples of such single-ring carbocyclyls include cyclopropanyl, cyclobutanyl, cyclopentyl, cyclopentenyl, cyclopentadienyl, cyclohexyl, cyclohexenyl, cyclohexadienyl, and phenyl. A carbocyclyl alternatively may be 2 or 3 rings Ufsed together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluoreneyl, decalinyl, and norpinanyl.

The term “cycloalkyl” (alone or in combination with another term(s)) means a saturated cyclic hydrocarbyl containing from 3 to 14 carbon ring atoms. A cycloalkyl may be a single carbon ring, which typically contains from 3 to 6 carbon ring atoms. Examples of single-ring cycloalkyls include cyclopropyl (or “cyclopropanyl”), cyclobutyl (or “cyclobutanyl”), cyclopentyl (or “cyclopentanyl”), and cyclohexyl (or “cyclohexanyl”). A cycloalkyl alternatively may be 2 or 3 carbon rings fused together, such as, decalinyl or norpinanyl.

The term “aryl” (alone or in combination with another term(s)) means an aromatic carbocyclyl containing from 6 to 14 carbon ring atoms. Examples of aryls include phenyl, naphthalenyl, and indenyl.

In some instances, the number of carbon atoms in a hydrocarbyl (e.g., alkyl, alkenyl, alkynyl, or cycloalkyl) is indicated by the prefix “Cₓ-C_{y}-” wherein x is the minimum and y is the maximum number of carbon atoms in the substituent. Thus, for example, “C₁-C₆-alkyl” refers to an alkyl containing from 1 to 6 carbon atoms. Illustrating further, C₃-C₆-Cycloalkyl means a saturated hydrocarbyl ring containing from 3 to 6 carbon ring atoms.

The term “hydrogen” (alone or in combination with another term(s)) means a hydrogen radical, and may be depicted as —H.

The term “hydroxy” (alone or in combination with another term(s)) means —OH.

The term “nitro” (alone or in combination with another term(s)) means —NO₂.

The term “cyano” (alone or in combination with another term(s)) means —CN, which also may be depicted as:

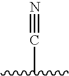

The term “azido” (alone or in combination with another term(s)) means —N₃.

The term “benzodioxolyl” (alone or in combination with another term(s)) can be depicted as

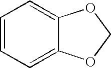

The term “keto” (alone or in combination with another term(s)) means an oxo radical, and may be depicted as ═O.

The term “carboxy” (alone or in combination with another term(s)) means —C(O)—OH, which also may be depicted as:

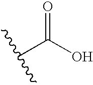

The term “amino” (alone or in combination with another term(s)) means —NH₂. The term “monosubstituted amino” (alone or in combination with another term(s)) means an amino wherein one of the hydrogen radicals is replaced by a non-hydrogen substituent. The term “disubstituted amino” (alone or in combination with another term(s)) means an amino wherein both of the hydrogen atoms are replaced by non-hydrogen substituents, which may be identical or different.

The term “cyclic amino” (alone or in combination with another term(s)) means a heterocyclyl moiety comprising at least one nitrogen ring atom, with the remaining ring atoms being carbon and optionally nitrogen. Examples of such moieties include piperidinyl and piperazinyl groups.

The term “halogen” (alone or in combination with another term(s)) means a fluorine radical (which may be depicted as —F), chlorine radical (which may be depicted as —Cl), bromine radical (which may be depicted as —Br), or iodine radical (which may be depicted as —I). Typically, a fluorine radical or chlorine radical is preferred, with a fluorine radical often being particularly preferred.

If a substituent is described as being “substituted”, a non-hydrogen radical is in the place of a hydrogen radical on, for example, a carbon or nitrogen of the substituent. Thus, for example, a substituted alkyl substituent is an alkyl substituent wherein at least one non-hydrogen radical is in the place of a hydrogen radical on the alkyl substituent. To illustrate, monofluoroalkyl is alkyl substituted with a fluoro radical, and difluoroalkyl is alkyl substituted with two fluoro radicals. It should be recognized that if there are more than one substitutions on a substituent, each non-hydrogen radical may be identical or different (unless otherwise stated).

If a substituent is described as being “optionally substituted”, the substituent is either (1) substituted, or (2) not substituted. Where the members of a group of substituents are described generally as being optionally substituted, any atom capable of substitution in each member of such group may be (1) substituted, or (2) not substituted. Such a characterization contemplates that some members of the group are not substitutable. Atoms capable of substitution include, for example, carbon bonded to at least one hydrogen, oxygen bonded to at least one hydrogen, sulfur bonded to at least one hydrogen, or nitrogen bonded to at least one hydrogen. On the other hand, hydrogen alone, halogen, oxo, and cyano do not fall within the definition of being capable of substitution.

This specification uses the terms “substituent” and “radical” interchangeably.

The prefix “halo” indicates that the substituent to which the prefix is attached is substituted with one or more independently selected halogen radicals. For example, haloalkyl means an alkyl wherein at least one hydrogen radical is replaced with a halogen radical. Examples of haloalkyls include chloromethyl, 1-bromoethyl, fluoromethyl, difluoromethyl, trifluoromethyl, 1,1,1-trifluoroethyl, and the like. Illustrating further, “haloalkoxy” means an alkoxy wherein at least one hydrogen radical is replaced by a halogen radical. Examples of haloalkoxy substituenits include chloromethoxy, 1-bromoethoxy, fluoromethoxy, difluoromethoxy, trifluoromethoxy (also known as “perfluoromethyoxy”), 1,1,1,-trifluoroethoxy, and the like. It should be recognized that if a substituent is substituted by more than one halogen radical, those halogen radicals may be identical or different (unless stated otherwise).

The prefix “perhalo” indicates that every hydrogen radical on the substituent to which the prefix is attached is replaced with independently selected halogen radicals, i.e., each hydrogen radical on the substituent is replaced with a halogen radical. If all the halogen radicals are identical, the prefix typically will identify the halogen radical. Thus, for example, the term “perfluoro” means that every hydrogen radical on the substituent to which the prefix is attached is substituted with a fluorine radical. To illustrate, the term “perfluoroalkyl” means an alkyl wherein a fluorine radical is in the place of each hydrogen radical. Examples of perfluoroalkyl substituents include trifluoromethyl (—CF₃), perfluorobutyl, perfluoroisopropyl, perfluorododecyl, perfluorodecyl, and the like. To illustrate further, the term “perfluoroalkoxy” means an alkoxy wherein each hydrogen radical is replaced with a fluorine radical. Examples of perfluoroalkoxy substituents include trifluoromethoxy (—O—CF₃), perfluorobutoxy, pertfluoroisopropoxy, perfluorododecoxy, perfluorodecoxy, and the like.

The term “carbonyl” (alone or in combination with another term(s)) means —C(O)—, which also may be depicted as:

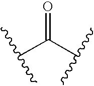

This term also is intended to encompass a hydrated carbonyl substituent, i.e., —C(OH)₂—.

The term “aminocarbonyl” (alone or in combination with another term(s)) means C(O)—NH₂, which also may be depicted as:

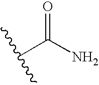

The term “oxy” (alone or in combination with another term(s)) means an ether substituent, and may be depicted as —O—.

The term “alkoxy” (alone or in combination with another term(s)) means an alkylether, i.e., —O-alkyl. Examples of such a substituent include methoxy (—O—CH₃), ethoxy, n-propoxy, isopropoxy, n-butoxy, iso-butoxy, sec-butoxy, tert-butoxy, and the like.

The term “alkylcarbonyl” (alone or in combination with another term(s)) means —C(O)-alkyl. For example, “ethylcarbonyl” may be depicted as:

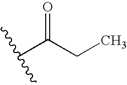

The term “alkoxycarbonyl” (alone or in combination with another term(s)) means —C(O)—O-alkyl. For example, “ethoxycarbonyl” may be depicted as:

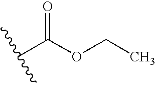

The term “carbocyclylcarbonyl” (alone or in combination with another term(s)) means —C(O)-carbocyclyl. For example, “phenylcarbonyl” may be depicted as:

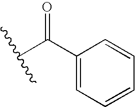

Similarly, the term “heterocyclylcarbonyl” (alone or in combination with another term(s)) means —C(O)-heterocyclyl.
The term “carbocyclylalkylcarbonyl” (alone or in combination with another term(s)) means —C(O)-alkyl-carbocyclyl. For example, “phenylethylcarbonyl” may be depicted as:

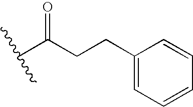

Similarly, the term “heterocyclylalkylcarbonyl” (alone or in combination with another term(s)) means —C(O)-alkyl-heterocyclyl.

The term “carbocyclyloxycarbonyl” (alone or in combination with another term(s)) means —C(O)—O-carbocyclyl. For example, “phenyloxycarbonyl” may be depicted as:

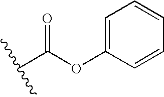

The term “carbocyclylalkoxycarbonyl” (alone or in combination with another term(s)) means —C(O)—O-alkyl-carbocyclyl. For example, “phenylethoxycarbonyl” may be depicted as:

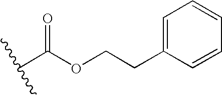

The term “thiol” or “thia” (alone or in combination with another term(s)) means a thiaether, i.e., an ether substituent wherein a divatent sulfur atom is in the place of the ether oxygen atom. Such a substituent may be depicted as —S—. This, for example, “alkyl-thio-alkyl” means alkyl-5-alkyl.

The term “thiol” or “sulfhydryl” (alone or in combination with another term(s)) means a sulfhydryl, and may be depicted as —SH.

The term “sulfonyl” (alone or in combination with another term(s)) means —S(O)₂—, which also may be depicted as:

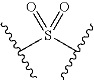

Thus, for example, “alkyl-sulfonyl-alkyl” means alkyl-S(O)₂-alkyl.

The term “aminosulfonyl” (alone or in combination with another term(s)) means —S(O)₂—NH₂, which also may be depicted as:

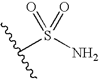

The term “heterocyclyl” (alone or in combination with another term(s)) means a saturated (i.e., “heterocycloalkyl”), partially saturated, or heteroaryl ring structure containing a total of 3 to 14 ring atoms. At least one of the ring atoms is a heteroatom (i.e., oxygen, nitTogen, or sulfur), with the remaining ring atoms being independently selected from the group consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, dihydrofurnayl, tetradydrofurnayl, thiophenyl (also known as “thiofuranyl” or “thienyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathliazolyl, oxadiazolyl (including 1,2,3-oxadiazolyl, 1,2,4-oxadiazolyl (also known as “azoximyl”), 1,2,5-oxadiazolyl (also known as “furazanyl”), and 1,3,4-oxadiazolyl), oxatriazolyl (including 1,2,3,4-oxatriazolyl and 1,2,3,5-oxatriazolyl), dioxazolyl (including 1,2,3-dioxazolyl, 1,2,4-dioxazolyl, 1,3,2-dioxazolyl, and 1,3,4-dioxazolyl), oxathiolanyl, pyranyl (including 1,2-pyranyl and 1,4-pyranyl), dihydropyranyl, pyridinyl, piperidinyl, diazinyl (including pyridazinyl (also known as “1,2-diazinyl”), pyrimidinyl (also known as “1,3-diazinyl”), and pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, and 1,4-oxazinyl), isoxazinyl (including o-isoxazinyl and p-isoxazinyl), oxazolidinyl, isoxazolidinyl, oxathiazinyl (including 1,2,5-oxathiazinyl and 1,2,6-oxathiazinyl), oxadiazinyl (including 1,4,2-oxadiazinyl and 1,3,5,2-oxadiazinyl), morpholinyl, azepinyl, oxepinyl, thiepinyl, and diazepinyl.

A heterocyclyl alternatively may be 2 or 3 rings fused together, such as, for example, indolizinyl, pyrindinyl, pyranopyrrolyl, 4H-quinolizinyl, purinyl, pyridopyridinyl (including pyrido[3,4-b]-pyridinyl, pyrido[3,2-b]-pyridinyl, pyrido[4,3-b]-pyridinyl, and naphthyridinyl), and pteridinyl. Other examples of fused-ring heterocyclyls include benzo-fused heterocyclyls, such as indolyl, isoindolyl, indoleninyl (also known as “pseudoindolyl”), isoindazolyl (also known as “benzpyrazolyl”), benzazinyl (including quinolinyl (also known as “1-benzazinyl”) and isoquinolinyl (also known as “2-benzazinyl”)), phthalazinyl, quinoxalinyl, benzodiazinyl (including cinnolinyl (also known as “1,2-benzodiazinyl”) and quinazolinyl (also known as “1,3-benzodiazinyl”)), benzopyranyl (including chromenyl and isochromenyl), benzothiopyranyl (also known as thiochromenyl), benzoxazolyl, indoxazinyl (also known as “2-benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl”, “thionaphthenyl”, or “benzothiofuranyl”), isobenzothienyl (also known as “isobenzothiophenyl”, “isothionaphthenyl”, or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, and 3,1,4-benzoxazinyl), benzisoxazinyl (including 1,2-benzisoxazinyl and 1,4-benzisoxazinyl), tetrahydroisoquinolinyl, carbazolyl, xanthenyl, and acridinyl.

The term “2-fused-ring” heterocyclyl (alone or in combination with another term(s)) means a saturated, partially saturated, or heteroaryl containing 2 fused rings. Examples of 2-fused-ring heterocyclyls include indolizinyl, pyrindinyl, pyranopyrrolyl, 4H-quinolizinyl, purinyl, pyridopyridinyl, pteridinyl, indolyl, isoindolyl, indoleninyl, isoindazolyl, benzazinyl, phthalazinyl, quinoxalinyl, quinazolinyl, benzodiazinyl, benzopytanyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzotbiadiazolyl, benzimidazolyl, benzotriazolyl, benzoxazinyl, benzisoxazinyl, and tettahydroisoquinolinyl.

The term “heteroaryl” (alone or in combination with another term(s)) means an aromatic heterocyclyl containing from 5 to 14 ring atoms. A heteroaryl may be a single ring or 2 or 3 fused rings. Examples of heteroaryl substituents include 6-membered ring substituents such as pyridinyl, pyrazinyl, pyrimidinyl, pyridazinyl, and 1,3,5-, 1,2,4-, and 1,2,3-triazinyl; 5-membered ring substituents such as imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, einnolinyl, and quinazolinyl.

A carbocyclyl or heterocyclyl can optionally be substituted with, for example, one or more substituents independently selected from the group consisting of halogen, hydroxy, carboxy, keto, alkyl, alkoxy, alkoxyalkyl, alkylcarbonyl (also known as “alkanoyl”), aryl, arylalkyl, arylalkoxy, arylalkoxyalkyl, arylalkoxycarbonyl, cycloalkyl, cycloalkylalkyl, cycloalkylalkoxy, cycloalkylalkoxyalkyl, and cycloalkylalkoxycarbonyl. More typically, a carbocyclyl or heterocyclyl may optionally be substituted with, for example, one or more substituents independently selected from the group consisting of halogen, hydroxy, carboxy, keto, C₁-C₆-alkyl, C₁-C₆-alkoxy, C₁-C₆-alkoxy-C₁-C₆-alkyl, C₁-C₆-alkylcarbonyl, aryl, aryl-C₁-C₆-alkyl, aryl-C₁-C₆-alkoxy, aryl-C₁-C₆-alkoxy-C₁-C₆-alkyl, aryl-C₁-C₆-alkoxycarbonyl, cycloalkyl, cycloalkyl-C₁-C₆-alkyl, cycloalkyl-C₁-C₆-alkoxy, cycloalkyl-C₁-C₆-alkoxy-C₁-C₆-alkyl, and cycloalkyl-C₁-C₆-alkoxycarbonyl. The alkyl, alkoxy, alkoxyalkyl, alkylcarbonyl, aryl, arylalkyl, arylalkoxy, arylalkoxyalkyl, or arylalkoxycarbonyl substituent(s) optionally may further be substituted with, for example, one or more halogen. The aryls or cycloalkyls are typically single-ring substituents containing from 3 to 6 ring atoms, and more typically from 5 to 6 ring atoms.

An aryl or heteroaryl can optionally be substituted with, for example, one or more substituents independently selected from the group consisting of halogen, hydroxy, cyano, amino, thiol, carboxy, amino, aminocarbonyl, aminoalkyl, alkyl, alkylthio, carboxyalkylthio, alkylcarbonyl, alkylcarbonyloxy, alkoxy, alkoxyalkyl, alkoxycarbonyl, alkoxycarbonylalkoxy, alkoxyalkylthio, alkoxycarbonylalkylthio, carboxyalkoxy, alkoxycarbonylalkoxy, carbocyclyl, carbocyclylalkyl, carbocyclyloxy, carbocyclylthio, carbocyclylalkylthio, carbocyclylamino, carbocyclylalkylamino, carbocyclylcarbonylamino, carbocyclylcarbonyl, carbocyclylalkyl, carbocyclylcarbonyloxy, carbocyclyloxycarbonyl, carbocyclylalkoxycarbonyl, carbocyclyloxyalkoxycarbocyclyl, carbocyclylthioalkylthiocarbocyclyl, carbocyclylthioalkoxycarbocyclyl, carbocyclyloxyalkylthiocarbocyclyl, heterocyclyl, heterocyclylalkyl, heterocyclyloxy, heterocyclylthio, heterocyclylalkylthio, heterocyclylamino, heterocyclylalkylamino, heterocyclylcarbonylamino, heterocyclylcarbonyl, heterocyclylalkylcarbonyl, heterocyclyloxycarbonyl, heterocyclylcarbonyloxy, heterocyclylalkoxycarbonyl, heterocyclyloxyalkoxyheterocyclyl, heterocyclylthioalkylthioheterocyclyl, heterocyclylthioalkoxyheterocyclyl, and heterocyclyloxyalkylthioheterocyclyl. More typically, an aryl or heteroaryl may, for example, optionally be substituted with one or more substituents independently selected from the group consisting of halogen, hydroxy, cyano, amino, thiol, carboxy, amino, aminocarbonyl, amino-C₁-C₆-alkyl, C₁-C₆-alkyl, C₁-C₆-alkylthio, carboxy-C₁-C₆-alkylthio, C₁-C₆-alkylcarbonyl, C₁-C₆-alkylcarbonyloxy, C₁-C₆-alkoxy, C₁-C₆-alkoxy-C₁-C₆-alkyl, C₁-C₆-alkoxycarbonyl, C₁-C₆-alkoxycarbonyl-C₁-C₆-alkoxy, C₁-C₆-alkoxy-C₁-C₆-alkylthio, C₁-C₆-alkoxycarbonyl-C₁-C₆-alkylthio, carboxy-C₁-C₆-alkoxy, C₁-C₆-alkoxycarbonyl-C₁-C₆-alkoxy, aryl, aryl-C₁-C₆-alkyl, aryloxy, arylthio, aryl-C₁-C₆-alkylthio, arylamino, aryl-C₁-C₆-alkylamino, arylcarbonylamino, arylcarbonyl, aryl-C₁-C₆-alkylcarbonyl, arylcarbonyloxy, aryloxycarbonyl, aryl-C₁-C₆-alkoxycarbonyl, aryloxy-C₁-C₆-alkoxyaryl, arylthio-C₁-C₆-alkylthioaryl, arylthio-C₁-C₆-alkoxyaryl, aryloxy-C₁-C₆-alkylthioaryl, cycloalkyl, cycloalkyl-C₁-C₆-alkyl, cycloalkyloxy, cycloalkylthio, cycloalkyl-C₁-C₆-alkylthio, cycloalkylamino, cycloalkyl-C₁-C₆-alkylamino, cycloalkylcarrbonylamino, cycloalkylcarbonyl, cycloalkyl-C₁-C₆-alkylcarbonyl, cycloalkylcarbonyloxy, cycloalkyloxycarbonyl, cycloalkyl-C₁-C₆-alkoxycarbonyl, heteroaryl, heteroaryl-C₁-C₆-alkyl, heteroaryloxy, heteroarylthio, heteroaryl-C₁-C₆-alyl thio, heteroarylamino, heteroaryl-C₁-C₆-alkylamino, heteroarylcarbonylamino, heteroarylcarbonyl, heteroaryl-C₁-C₆-alkylcarbonyl, heteroaryloxycarbonyl, heteroarylcarbonyloxy, and heteroaryl-C₁-C₆-alkoxycarbonyl. Here, one or more hydrogen bound to a carbon in any such substituent may, for example, optionally be replaced with halogen. In addition, the cycloalkyl, aryl, and heteroaryl are typically single-ring substituents containing 3 to 6 ring atoms, and more typically 5 or 6 ring atoms.

A prefix attached to a multi-Component substituent only applies to the first component. To illustrate, the term “alkylcycloalkyl” contains two components: alkyl and cycloalkyl. Thus, the C₁-C₆-prefix on “C₁-C₆-alkylcycloalkyl” means that the alkyl component of the alkylcycloalkyl contains from 1 to 6 carbon atoms; the C₁-C₆-prefix does not describe the cycloalkyl component. To illustrate further, the prefix “halo” on haloalkoxyalkyl indicates that only the alkoxy component of the alkoxyalkyl substituent is substituted with one or more halogen radicals. If halogen substitution may alternatively or additionally occur on the alkyl component, the substituent would instead be described as “halogen-substituted alkoxyalkyl” rather than “haloalkoxyalkyl.” And finally, if the halogen substitution may only occur on the alkyl component, the substituent would instead be described as “alkoxyhaloalkyl.”

If substituents are described as being “independently selected” from a group, each substituent is selected independent of the other. Each substituent therefore may be identical to or different from the other substituent(s).

When words are used to describe a substituent, the rightmost-described component of the substituent is the component that has the free valence. To illustrate, benzene substituted with methoxyethyl has the following structure:

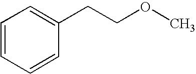

As can be seen, the ethyl is bound to the benzene, and the methoxy is the component of the substituent that is the component furthest from the benzene. As further illustration, benzene substituted with cyclohexanylthiobutoxy has the following structure:

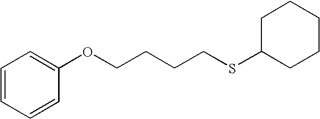

When words are used to describe a linking element between two other elements of a depicted chemical structure, the rightmost-described component of the substituent is the component that is bound to the left element in the depicted structure. To illustrate, if the chemical structure is X-L-Y and L is described as methylcyclohexanylethyl, then the chemical would be X-ethyl-Cyclohexanyl-methyl-Y.

When a chemical formula is used to describe a substituent, a hanging dash in the formula indicates a free valence. To illustrate, benzene substituted with —C(O)—OH has the following structure:

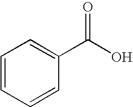

When a chemical formula is used to describe a linking element between two other elements of a depicted chemical structure, the leftmost dash of the substituent indicates the portion of the substituent that is bound to the left element in the depicted structure. The rightmost dash, on the other hand, indicates the portion of the substituent that is bound to the right element in the depicted structure. To illustrate, if the depicted chemical structure is X-L-Y and L is described as —C(O)—N(H)—, then the chemical would be:

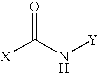

The term “pharmaceutically acceptable” is used adjectivally in this specification to mean that the modified noun is appropriate for use as a pharmaceutical product or as a part of a pharmaceutical product.

The term “ambient pressure” means about 1 atmosphere.

The terms “room temperature” and “ambient temperature” mean a temperature of from about 20 to about 25° C.

The abbreviation “DMF” means dimethylformamide (also called “N,N-dimethylformamide”).

The abbreviation “DMSO” means dimethyl sulfoxide.

The abbreviation “THF” means tetrahydrofuran.

The abbreviation “BOC” means t-butyloxycarbonyl.

With reference to the use of the words “comprise” or “comprises” or “comprising” in this specification, Applicants note that unless the context requires otherwise, those words are to be interpreted inclusively, rather than exclusively, and that Applicants intend each of those words to be so interpreted in construing this specification.

The following examples are merely illustrative, and not limited to this disclosure in any way.

### EXAMPLES

### Examples/Methods of Preparation and Synthesis

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

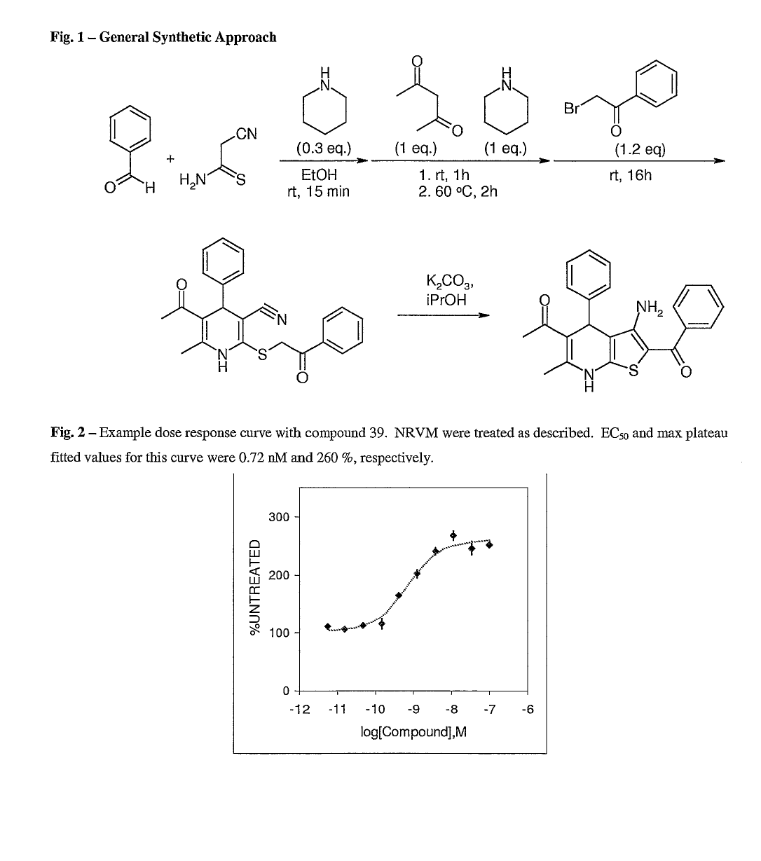

FIG. 1 show s a general synthetic approach.

Certain starting materials of Formula III below, useful for the preparation of compounds of Formula I and Formula ft, are known in the art:

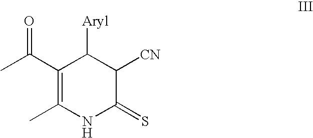

wherein aryl phenyl, furan-2-yl and thiophen-2-yl and substitutions thereon.

Detailed preparations for these starting materials are found, for aryl phenyl, in Krauze et al. (1984); Krauze et al. (1988); Krauze et al. (1991); Krauze et al. (1998); Krauze and Dudurs (2000); Tirzite et al. (2002); Sharanin et al. (1985); Sharanin et al. (1986); and for aryl=furan-2-yl and thiophen-2-yl in Attaby et al. (1996).

The compounds of the present invention can be prepared according to the following methods.

### Example A

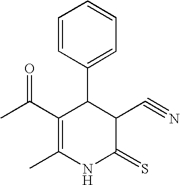

3 g of 2,4-pentane dione (30 mmol) and 3.18 g of benzaldehyde (30 mmol) are dissolved in 25 ml of ethanol, stirred magnetically at ambient temperature and to this is added 850 mg of piperidine (10 mmol). The mixture is left to stir at ambient temperature for 30 minutes (the colorless solution turns yellow during this time). The reaction is cooled to 15° C. and 3.05 g of thioacetamide (30 mmol) is added (the color changes to light brown-red) followed by an additional 2.6 g of piperidine (30 mmol). After approximately 30 minutes a solid precipitate forms. The temperature is maintained at 15-20° C. for a total of 1 hour and the solid piperidine salt of 5-acetyl-6-methyl-4-phenyl-2-thioxo-1,2,3,4-tetrahydro-pyridine-3-carbonitrile is filtered off on a sintered glass funnel and washed with diethyl ether. This solid piperidine salt of 5-acetyl-6-methyl-4-phenyl-2-thioxo-1,2,3,4-tetrahydro-pyridine-3-carbonitrile is used as is.

### Example B

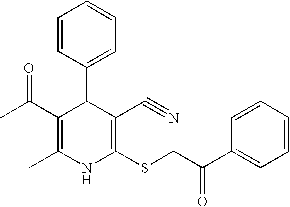

2-Bromoacetophenone (25 mmol) is dissolved in 50 ml of methanol and the solution stirred magnetically at 10° C. To this is added 7.65 g of the piperidine salt from example A (22 mmol). The salt dissolves and a yellow solid precipitates. After 1 hour this solid is filtered off and washed with diethyl ether. The solid is slurried between water and ethyl acetate (using magnetic stirring) and the aqueous layer acidified with 1 N HCl (to a pH of 1-2). The solid slowly goes into the ethyl acetate (and the color is an intense yellow). The layers are separated, washed with a saturated sodium chloride solution, dried with MgSO₄, filtered and concentrated with heating under reduced pressure to a small volume. Adding hexane causes a yellow solid to precipitate. The solid is dried under vacuum to obtain 5-acetyl-6-methyl-2-(2-oxo-2-phenyl-ethylsulfanyl)-4-phenyl-1,4 dihydro-pyridine-3-carbonitrile MS (M−1): 387.1. The material can also further purified by flash chromatography (ethyl acetate:hexane 1:1).

### Example C

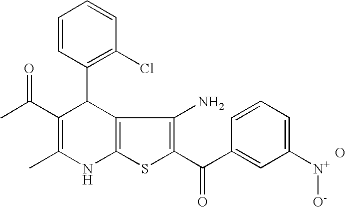

2 g of 5-acetyl-4-(2-chloro-phenyl)-6-methyl-2-[2-(3-nitro-phenyl)-2-oxo-ethylsulfanyl]-1,4 dihydro-pyridine-3-carbonitrile (4.27 mmol) are added to 36 ml ethanol at room temperature, 238 mg potassium hydroxide (4.27 mmol) in 0.75 ml water and 1 ml of ethanol is added dropwise. The system is stirred for 2 hours. Ice/H₂O is added to give an oily emulsion which is extracted with ethyl acetate. The solution is dried, filtered and removed solvent under reduced pressure. The material is crystallized by the addition of diethyl ether/hexane, filtered and washed with hexane to give the desired 1-[3-amino-4-(2-chloro-phenyl)-6-methyl-2-(3-nitro-benzoyl)-4,7-dihydro-thieno[2,3-b]pyridin-5-yl]-ethanone compound 4) MS (M+1): 468.

### Example D

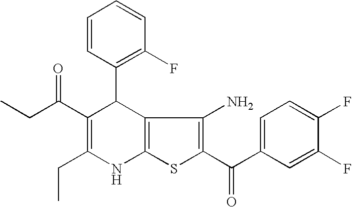

2-Fluorobenzaldehyde (1.94 g, 15.6 mmol), 2-cyanothioacetamide (1.56 g, 15.6 mmol) and piperidine (0.16 ml, 1.60 mmol) are dissolved in ethanol (30 ml). The mixture is stirred at ambient temperature for 30 minutes. 3,5-Heptanedione (2.0 g, 15.6 mmol) is added dropwise to the reaction mixture followed by the addition of another portion of piperidine (1.87 ml, 18.7 mmol). The reaction mixture is stirred at ambient temperature for 1 hour then heated in a 60° C. heating bath for 2 hours. The reaction is cooled in a water-ice bath. 2-Bromo-3′,4′ difluoroacetophenone (3.85 g, 16.4 mmol) and potassium carbonate (4.30 g, 31.2 mmol) are added to the reaction mixture in sequence. The reaction is removed from the cooling bath and stirred at ambient temperature overnight. The crude reaction is partitioned between ethyl acetate (500 ml) and water (200 ml). The organic phase washed with brine (100 ml) and dried over sodium sulfate. Solvents are removed in vaccuo and the residue purified by flash chromatography (silica gel, 15-35% ethyl acetate in hexanes) to obtain the desired 1-[3-amino-2-(3,4 difluoro-benzoyl)-6-ethyl-4-(2 fluoro-phenyl)-4,7 dihydro-thieno[2,3-b]pyridine-5-yl]propan-1-one (compound 108) as a yellow solid. MS (M+1): 471.1.

### Example E

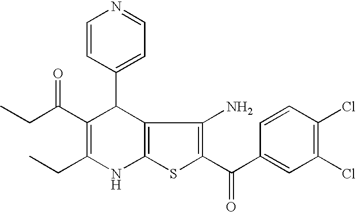

2-Cyanothioactamide (1.0 g, 10 mmol), 4-pyridinecarboxaldehyde (0.95 ml, 10 mmol), and piperidine (0.1 ml, 1 mmol) are dissolved in ethanol (20 ml). The mixture is stirred at ambient temperature for 15 minutes. 3,5-Heptanedione (1.35 ml, 10 mmol) is added dropwise to the reaction mixture followed by an additional portion of piperidine (1.2 ml, 12 mmol). The reaction is stirred at ambient temperature for 1 hour then heated in a 60° C. heating bath for 2 hours. The reaction is cooled in a water-ice bath. 3,4 dichlorophenacyl bromide (2.68 g, 10 mmol) and potassium carbonate (2.76 g, 20 mmol) are added to the reaction in sequence. The reaction is removed from the cooling bath and stirred overnight at ambient temperature. The crude reaction is diluted with water and washed with ethyl acetate. The organic extract is dried over sodium sulfate. Solvents are removed in vaccuo and the residue purified by flash chromatography (silica gel, ethyl acetate:hexanes=1:1) to obtain the product, 1-[3-amino-2-(3,4 dichloro-benzoyl)-6-ethyl-4 pyridin-4-yl-4,7 dihydro-thieno[2,3-b]pyridin-5-yl]-propan-1-one (compound 104), as a yellow solid. MS (M+1): 486.1.

### Example F

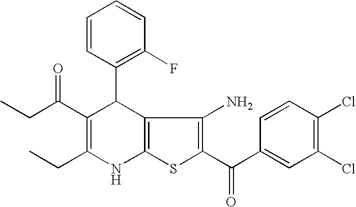

2-Cyanothioactamide (5.02 g, 50.1 mmol), 2-fluorobenzaldehyde (5.30 ml, 50.0 mmol), and piperidine (0.1 ml 1 mmol) are dissolved in ethanol (50 ml). The mixture is stirred at ambient temperature for 15 minutes. 3,5-Heptanedione (6.8 ml, 50.2 mmol) is added dropwise to the reaction mixture followed by an additional portion of piperidine (5.0 ml, 50 mmol). The reaction is stirred at ambient temperature for 30 minutes then heated in a 60° C. heating bath for 1 hour. The reaction is cooled in a water-ice bath. 3,4 dichlorophenacyl bromide (13.50 g, 50.4 mmol) is added and the reaction is maintained at lower temperature in a water-ice bath and stirred for 2 hours. Potassium carbonate (2.21 g, 16.0 mmol) is added to the reaction. The reaction is removed from the cooling bath and stirred for 18 hours at ambient temperature. The product is filtered and the residue washed with water and cold ethanol and dried under vacuum to give the expected 1-[3-amino-2-(3,4-dichloro-benzoyl)-6-ethyl-4-(2-fluoro-phenyl)-4,7-dihydro-thieno[2,3b]pyridin-5-yl]-propan-1-one (compound 39) as a yellow solid. MS (M+1): 503.1.

The following are prepared using the methods of examples A-F:

TABLE 1

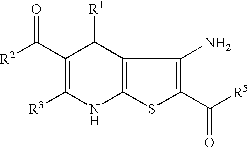

| Comp | EC50, M | Max Plateau | R² | R¹ | R⁵ | MS Found |
|---|---|---|---|---|---|---|
| 1 | | | Me | 2 Pyr | Ph | 390 (M + 1) |
| 2 | 1.77E−07 ± 3.72E−08 | 207.74 ± 23.00 | Me | Ph | 3,4 diClPh | 457 (M + 1) |
| 3 | 7.97E−07 ± 3.94E−07 | 250.88 ± 32.62 | Me | Ph | Ph | 389 (M + 1) |
| 4 | 8.57E−08 ± 1.60E−07 | 244.20 ± 37.25 | Me | 2 ClPh | 3 NO₂Ph | 468 (M + 1) |
| 5 | 2.33E−06 ± 4.63E−07 | 286.43 ± 42.04 | Me | Ph | 3 Pyr | 390 (M + 1) |
| 6 | 4.89E−08 ± 5.23E−09 | 289.34 ± 70.69 | Me | Ph | 3 BrPh | 467 (M + 1) |
| 7 | | | Me | Ph | 3 MeOPh | 419 (M + 1) |
| 8 | 7.97E−07 ± 2.05E−07 | 172.29 ± 9.66 | Me | Ph | 3,4 diMePh | 417 (M + 1) |
| 9 | 1.00E−03 ± 0.00E+00 | 100.00 ± 0.00 | Me | 4 CO₂HPh | 3 NO₂Ph | 478 (M + 1) |
| 10 | 2.68E−06 ± 2.10E−07 | 210.00 ± 36.06 | Me | Ph | 4 Pyr | 390 (M + 1) |
| 11 | 2.82E−08 ± 1.10E−08 | 264.94 ± 39.68 | Et | Ph | Ph | 417 (M + 1) |
| 12 | 2.82E−08 ± 1.10E−08 | 264.94 ± 39.68 | Me | Ph | F Ph | 407.1 (M + 1) |
| 13 | 8.25E−08 ± 5.35E−08 | 2.33.83 ± 33.69 | Me | 2 ClPh | 3,4 diClPh | 491 (M + 1) |
| 14 | 3.03E−06 ± 9.29E−07 | 182.73 ± 26.95 | Me | 4 MeOPh | Ph | 419 (M + 1) |
| 15 | 3.34E−04 ± 5.77E−04 | 126.50 ± 24.27 | Me | 3 MeOPh | Ph | 419 (M + 1) |
| 16 | 4.29E−06 ± 6.65E−07 | 163.33 ± 32.15 | Me | Ph | 2-Pyr | 390 (M + 1) |
| 17 | | | Me | 2 MeOPh | 3,4 diClPh | 487 (M + 1) |
| 18 | 8.76E−07 ± 3.32E−07 | 215.25 ± 44.08 | Me | 2 MeOPh | Ph | 419.0 (M + 1) |
| 19 | 3.39E−06 ± 4.06E−07 | 260.00 ± 36.06 | Me | 4 Pyr | Ph | 390 (M + 1) |
| 20 | 3.35E−04 ± 5.76E−04 | 127.35 ± 23.71 | Me | 3 Pyr | Ph | 390 (M + 1) |
| 21 | 5.83E−07 ± 3.09E−08 | 193.20 ± 49.26 | Me | 4 ClPh | Ph | 423 (M + 1) |
| 22 | 2.07E−06 ± 2.12E−06 | 174.35 ± 23.39 | Me | 4 BrPh | 3,4 diClPh | 535 (M + 1) |
| 23 | 3.39E−07 ± 2.02E−07 | 277.53 ± 33.46 | Me | 4 HOPh | 3,4 diClPh | 473.1 (M + 1) |
| 24 | 2.73E−07 ± 1.32E−07 | 260.35 ± 43.53 | Me | 2 FPh | Ph | 407 (M + 1) |
| 25 | 1.00E−03 ± 0.00E+00 | 100.00 ± 0.00 | Me | 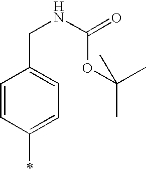 | 3,4 diClPh | 586.2 (M + 1) |
| 26 | 9.43E−07 ± 7.65E−07 | 185.33 ± 55.04 | Me | cyclohexyl | Ph | 395 (M + 1) |
| 27 | 8.64E−08 ± 1.50E−08 | 220.13 ± 23.10 | Me | 2 FPh | 3,4 diClPh | 475 (M + 1) |
| 28 | | | Me | Ph | 3 N₃Ph | 430 (M + 1) |
| 29 | 1.55E−08 ± 1.48E−08 | 281.88 ± 32.78 | Me | 2 Cl, 4 HOPh | 3,4 diClPh | 509 (M + 1) |
| 30 | 1.00E−03 ± 0.00E+00 | 100.00 ± 0.00 | Ph | Ph | Ph | 513 (M + 1) |
| 31 | 3.34E−04 ± 5.77E−04 | 121.08 ± 18.32 | Me | 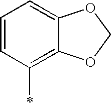 | 3,4 diClPh | 501 (M + 1) |
| 32 | 1.00E−03 ± 0.00E+00 | 100.00 ± 0.00 | Me | 4 tBuPh | 3,4 diClPh | 513 (M + 1) |
| 33 | 1.95E−06 ± 2.25E−06 | 143.15 ± 21.22 | Me | 3,5 diClPh | 3,4 diClPh | 525 (M + 1) |
| 34 | 1.25E−06 ± 2.09E−07 | 184.69 ± 15.76 | Me | 2 HOPh | 3,4 diClPh | 473 (M + 1) |
| 35 | | | Me | Ph | 4 N₃Ph | 430 (M + 1) |
| 36 | 6.18E−09 ± 2.48E−09 | 256.56 ± 24.93 | Et | Ph | 3,4 diClPh | 485 (M + 1) |
| 37 | 2.81E−07 ± 7.02E−08 | 227.92 ± 12.46 | Me | 4 FPh | Ph | 406.9 (M + 1) |
| 38 | 1.61E−08 ± 3.28E−08 | 288.93 ± 52.81 | Et | 2 ClPh | 3,4 diClPh | 519.0 (M + 1) |
| 39 | 1.71E−08 ± 3.90E−08 | 272.61 ± 37.32 | Et | 2 FPh | 3,4 diClPh | 503.1 (M + 1) |
| 40 | 1.44E−08 ± 2.85E−09 | 244.87 ± 7.92 | Et | 2 F,6 ClPh | 2,4 diClPh | 537 (M + 1) |
| 41 | 3.69E−07 ± 7.73E−08 | 178.47 ± 7.82 | Me | 2 Thiophene | 3,4 diClPh | 463 (M + 1) |
| 42 | 4.59E−06 ± 1.91E−06 | 160.56 ± 20.02 | Me | 2 Pyrole | 3,4 diClPh | 446 (M + 1) |
| 43 | | | Me | 4-MePh | Ph | 403 (M + 1) |
| 44 | 1.72E−06 ± 2.91E−07 | 186.67 ± 11.55 | Me | 4 CF₃Ph | Ph | 456.9 (M + 1) |
| 45 | 3.34E−04 ± 5.77E−04 | 108.63 ± 9.14 | Me | 4 MePh | 3,4 diClPh | 470.9 (M + 1) |
| 46 | 1.00E−03 ± 1.50E−11 | 98.33 ± 4.08 | iPr | Ph | 3,4 diClPh | 513 (M + 1) |
| 47 | 1.16E−07 ± 1.53E−08 | 173.47 ± 25.64 | Me | 3,4 diClPh | 3,4 diClPh | 526.8 (M + 1) |
| 48 | 5.11E−06 ± 2.24E−06 | 190.81 ± 37.09 | Me | 4 CF₃Ph | 3,4 diClPh | 524.9 (M + 1) |
| 49 | 1.44E−08 ± 4.03E−09 | 299.18 ± 42.31 | Et | Ph | 3 FPh | 435.2 (M + 1) |
| 50 | 1.61E−07 ± 2.77E−08 | 190.79 ± 27.94 | Me | 4 FPh | 3,4 diClPh | 474.8 (M + 1) |
| 51 | | | Me | 2,4 diFPh | 3,4 diClPh | 492.8 (M + 1) |
| 52 | 3.36E−04 ± 5.75E−04 | 122.73 ± 20.55 | Me | 2,4 diClPh | 3,4 diClPh | 526.8 (M + 1) |
| 53 | 5.81E−08 ± 1.23E−08 | 259.43 ± 26.30 | Et | 2 ClPh | 3,4 diClPh | 519.0 (M + 1) |
| 54 | 5.90E−10 ± 2.96E−10 | 267.44 ± 32.29 | Et | 3 ClPh | 3,4 diClPh | 519.0 (M + 1) |
| 55 | 4.52E−08 ± 2.97E−08 | 229.93 ± 37.33 | Me | 3,5 diFPh | 3,4 diClPh | 492.8 (M + 1) |
| 56 | 4.29E−08 ± 7.56E−09 | 293.74 ± 50.82 | Et | 2 MePh | 3,4 diClPh | 499.1 (M + 1) |
| 57 | 1.25E−06 ± 6.39E−07 | 246.00 ± 16.33 | Me | Ph | 2 ClPh | 423 (M + 1) |
| 58 | 1.29E−07 ± 1.14E−07 | 232.20 ± 56.57 | Et | 3 NO₂Ph | 3,4 diClPh | 530 (M + 1) |
| 59 | 4.25E−07 ± 3.23E−07 | 152.77 ± 11.42 | Et | 2 NO₂Ph | 3,4 diClPh | 530 (M + 1) |
| 60 | 1.00E−03 ± 0.00E+00 | 100.00 ± 0.00 | Et | 2 CF₃Ph | 3,4 diClPh | 553.1 (M + 1) |
| 61 | 9.71E−08 ± 1.13E−08 | 230.65 ± 55.61 | Me | Ph | 3 FPh | 407.1 (M + 1) |
| 62 | 3.61E−07 ± 8.90E−08 | 279.31 ± 67.94 | Me | Ph | 3 CO₂MePh | 447.1 (M + 1) |
| 63 | 2.08E−08 ± 2.41E−09 | 230.07 ± 45.59 | Et | 2,6 diFPh | 3,4 diClPh | 521.1 (M + 1) |
| 64 | 1.02E−08 ± 8.80E−09 | 281.39 ± 54.54 | Et | Ph | 2, 5 diClPh | 484.9 (M + 1) |
| 65 | 1.00E−03 ± 0.00E+00 | 100.00 ± 0.00 | Me | Ph | 3 CO₂HPh | 433.1 (M + 1) |
| 66 | 4.94E−09 ± 4.74E−09 | 306.19 ± 54.21 | Et | Ph | 3 ClPh | 451.1 (M + 1) |
| 67 | 1.21E−08 ± 3.85E−09 | 256.12 ± 53.66 | Et | Ph | 2 FPh | 435.2 (M + 1) |
| 68 | 2.33E−07 ± 1.40E−08 | 175.88 ± 14.54 | Et | 3 Pyr | Ph | 418.2 (M + 1) |
| 69 | 3.17E−08 ± 1.19E−08 | 221.36 ± 15.94 | Et | 2,6 diFPh | Ph | 453 (M + 1) |
| 70 | 5.17E−08 ± 3.36E−08 | 191.97 ± 13.09 | Et | Ph | 2,4 diClPh | 485 (M + 1) |
| 71 | 5.75E−08 ± 5.93E−08 | 223.30 ± 32.84 | Et | 2 FPh | 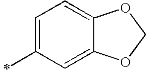 | 479 (M + 1) |
| 72 | 9.23E−09 ± 3.14E−09 | 223.99 ± 24.51 | Et | Ph | 3,4 diFPh | 453 (M + 1) |
| 73 | 8.92E−09 ± 1.97E−09 | 205.27 ± 38.78 | Et | Ph | 3,5 diCF₃Ph | 553 (M + 1) |
| 74 | 9.84E−09 ± 7.45E−09 | 262.81 ± 32.38 | Et | Ph | 3 CO₂MePh | 475.2 (M + 1) |
| 75 | 6.13E−08 ± 5.91E−09 | 226.71 ± 40.80 | Et | 3 Pyr | 3,4 diClPh | 486.1 (M + 1) |
| 76 | 2.42E−08 ± 1.90E−08 | 230.70 ± 23.22 | Et | Ph | 3,5 diFPh | 453.1 (M + 1) |
| 77 | 2.14E−06 ± 5.81E−07 | 220.00 ± 10.00 | Et | Ph | 3 CO₂HPh | 460.9 (M + 1) |
| 78 | 6.77E−09 ± 6.39E−09 | 269.77 ± 35.30 | Et | 2 FPh | Ph | 435.2 (M + 1) |
| 79 | 4.08E−08 ± 1.55E−08 | 241.90 ± 15.79 | Et | Ph | 4 FPh | 435.2 (M + 1) |
| 80 | 1.03E−07 ± 2.10E−08 | 226.13 ± 5.25 | Et | Ph | 4 ClPh | 451.1 (M + 1) |
| 81 | 3.50E−07 ± 3.61E−07 | 184.00 ± 15.51 | Et | 2 F,4 MeOPh | 3,4 diClPh | 533.1 (M + 1) |
| 82 | 1.48E−08 ± 1.39E−08 | 245.96 ± 30.73 | Et | Ph | 2 ClPh | 451.1 (M + 1) |
| 83 | 2.49E−10 ± 1.40E−10 | 274.88 ± 44.56 | Et | 2 Cl,4 HOPh | 3,4 diClPh | 535.0 (M + 1) |
| 84 | 5.40E−09 ± 9.18E−10 | 242.72 ± 19.34 | Et | 3 FPh | 3,4 diClPh | 503.1 (M + 1) |
| 85 | 3.33E−04 ± 5.77E−04 | 105.90 ± 6.18 | Et | 1 Naphthalenyl | 3,4 diClPh | 535.0 (M + 1) |
| 86 | 6.27E−07 ± 5.92E−07 | 131.26 ± 2.34 | Pr | Ph | 3,4 diClPh | 513.2 (M + 1) |
| 87 | 2.17E−07 ± 1.06E−07 | 187.66 ± 12.24 | Pr | 2 FPh | 3,4 diClPh | 531.2 (M + 1) |
| 88 | 3.35E−04 ± 5.75E−04 | 113.45 ± 11.92 | Et | 2 Naphthalenyl | 3,4 diClPh | 535.0 (M + 1) |
| 89 | 9.69E−09 ± 4.67E−09 | 230.09 ± 19.81 | Et | Ph | 3 Me,4 ClPh | 465 (M + 1) |
| 90 | 6.67E−04 ± 5.77E−04 | 300.00 ± 0.00 | Et | Ph | 3,5 di tBu, 4-OH Ph | 545 (M + 1) |
| 91 | 1.98E−07 ± 1.76E−07 | 143.39 ± 20.69 | Pr | 2 FPh | 2,4 diClPh | 531.3 (M + 1) |
| 92 | 5.05E−08 ± 1.63E−08 | 189.00 ± 25.40 | Pr | 2 FPh | 2,5 diClPh | 531.3 (M + 1) |
| 93 | | | Pr | Ph | 2,4 diClPh | 513.2 (M + 1) |
| 94 | 1.18E−07 ± 6.31E−08 | 153.83 ± 13.14 | Pr | Ph | 2,5 diClPh | 513.2 (M + 1) |
| 95 | 6.10E−07 ± 8.47E−09 | 147.01 ± 6.43 | Pr | Ph | 3,4 diFPh | 481.1 (M + 1) |
| 96 | 1.71E−08 ± 1.46E−08 | 254.87 ± 72.67 | Et | Ph | 3 NO₂ Ph | 462 (M + 1) |
| 97 | 1.64E−09 ± 6.26E−10 | 247.24 ± 38.88 | Et | 2 FPh | 3 NO₂ Ph | 480 (M + 1) |
| 98 | 4.99E−09 ± 2.99E−09 | 255.02 ± 32.01 | Et | 2 F, 4 Pyr | 3,4 diClPh | 504.2 (M + 1) |
| 99 | 5.23E−09 ± 4.25E−09 | 264.37 ± 33.84 | Et | 2 Cl, 4 Pyr | 3,4 diClPh | 522.2 (M + 1) |
| 100 | 5.73E−09 ± 4.62E−09 | 262.04 ± 31.65 | Et | 3,5 di FPh | 3,4 diClPh | 521.0 (M + 1) |
| 101 | 3.72E−09 ± 3.66E−10 | 230.49 ± 17.13 | Et | 3,5 di FPh | 3,4 diFPh | 489.3 (M + 1) |
| 102 | 4.18E−09 ± 1.91E−09 | 269.88 ± 36.90 | Et | 2 F, 4 Pyr | 3,4 diFPh | 472.1 (M + 1) |
| 103 | 1.05E−08 ± 2.80E−09 | 254.65 ± 31.26 | Et | 2 F, 4 Pyr | 3 Pyr | 437.1 (M + 1) |
| 104 | 2.48E−08 ± 5.94E−09 | 242.77 ± 33.52 | Et | 4 Pyr | 3,4 diClPh | 486.1 (M + 1) |
| 105 | 4.38E−09 ± 7.23E−10 | 273.18 ± 37.33 | Et | 3,5 diFPh | Ph | 453.1 (M + 1) |
| 106 | 8.93E−09 ± 2.97E−09 | 244.21 ± 29.53 | Et | Ph | 2-Br Ph | 495.0 (M + 1) |
| 107 | 1.67E−09 ± 1.03E−09 | 273.85 ± 29.06 | Et | 2 FPh | 2,6 diClPh | 503.0 (M + 1) |
| 108 | 4.02E−09 ± 1.96E−09 | 266.30 ± 58.14 | Et | 2 FPh | 3,4 diFPh | 471.1 (M + 1) |
| 109 | | | Et | Ph | 3 F, 2-CO₂Et Ph | 507.1 (M + 1) |
| 110 | 1.67E−07 ± 3.39E−08 | 118.50 ± 1.77 | Me | 3 FPh | 3,4 diClPh | 489.1 (M + 1) |
| 111 | 1.00E−03 ± 0.00E+00 | 233.33 ± 115.47 | E | Ph | 4 CO₂Me Ph | 426.4 (M + 1) |
| 112 | 7.73E−04 ± 3.93E−04 | 266.67 ± 57.74 | E | Ph | 4 CN Ph | 442.1 (M + 1) |

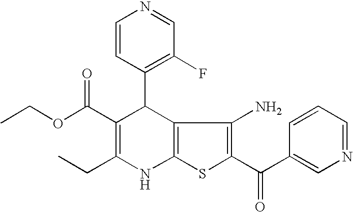

2-Cyanothioactamide (0.8 g, 8 mmol), 3 fluoro-4 pyridinecarboxaldehyde (0.8 ml, 8 mmol), and piperidine (0.09 ml, 0.8 mmol) are dissolved in ethanol (16 ml). The mixture is stirred at ambient temperature for 15 min. Ethyl propionylacetate (1.14 ml, 8 mmol) is added dropwise to the reaction mixture followed by an additional portion of piperidine (0.96 ml, 9.6 mmol). The reaction is stirred at ambient temperature for 1 hour then heated in a 60° C. heating bath for 2 hours. The reaction is cooled in a water-ice bath. 2-Bromo-1-pyridin-3-ylethan-1-one hydrobromide (2.25 g, 8 mmol) and potassium carbonate (3.32 g, 24 mmol) are added to the reaction in sequence. The reaction is removed from the cooling bath and stirred overnight at ambient temperature. The crude reaction is diluted with water and washed with ethyl acetate. The organic extract is dried over sodium sulfate and filtered. Solvents are removed in vaccuo and the residue purified by flash chromatography (silica gel, ethyl acetate:hexanes=1:1) to obtain the product, 3-amino-6-ethyl-4-(3 fluoro-pyridin-4-yl)-2-(pyridine-3-carbonyl)-4,7 dihydro-thieno[2,3-b]pyridine-5-carboxylic acid ethyl ester (compound 174), as a yellow solid. MS (M+1): 453.1.

The following esters are prepared using the methods of example G:

TABLE 2

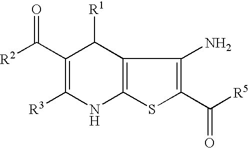

| | | Max | | | | | |
|---|---|---|---|---|---|---|---|
| Comp | EC50, M | Plateau | R² | R³ | R¹ | R⁵ | MS Found |
| 113 | 6.80E−08 ± | 256.38 ± | EtO | Me | Ph | 3,4 diClPh | 487 (M + 1) |
| | 4.21E−08 | 34.52 | | | | | |
| 114 | 1.79E−07 ± | 226.11 ± | EtO | Me | 4 NO₂Ph | Ph | 464.0 (M + 1) |
| | 4.02E−08 | 48.99 | | | | | |
| 115 | 5.21E−08 ± 3.74E−08 | 233.10 ± 15.43 | 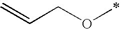 | Ph | Ph | 3,4 diClPh | 499.1 (M + 1) |
| 116 | 7.00E−08 ± | 107.57 ± | MeO | iPr | Ph | 3,4 diClPh | 501 (M + 1) |
| | 1.21E−08 | 0.78 | | | | | |
| 117 | | | EtO | Me | Ph | 3,4 diClPh | 488.5 (M + 1) |
| 118 | | | EtO | Me | Ph | 3,4 diClPh | 488.5 (M + 1) |
| 119 | 1.42E−07 ± | 234.93 ± | MeO(CH₂)₂O | Me | Ph | 3,4 diClPh | 517.2 (M + 1) |
| | 3.88E−08 | 57.72 | | | | | |
| 120 | 1.57E−07 ± | 140.79 ± | MeO | MeOCH₂ | Ph | 3,4 diClPh | 503.2 (M + 1) |
| | 3.47E−08 | 17.44 | | | | | |
| 121 | | | EtO | Me | Ph | 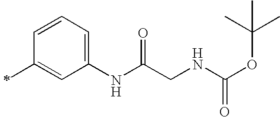 | 591.4 (M + 1) |
| 122 | | | EtO | Me | Ph | 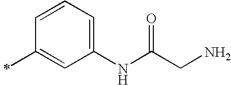 | 491.3 (M + 1) |
| 123 | | | EtO | Me | Ph | 4 N₃Ph | 460.2 (M + 1) |
| 124 | | | 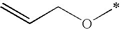 | Me | Ph | 4 N₃Ph | 470.2 (M + 1) |
| 125 | | | MeO | Me | Ph | 4 N₃Ph | 446.2 (M + 1) |
| 126 | 2.10E−07 ± 1.38E−07 | 193.06 ± 11.02 | 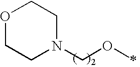 | Me | Ph | 3,4 diClPh | 572.2 (M + 1) |
| 127 | 5.40E−08 ± 2.42E−09 | 195.71 ± 29.42 | 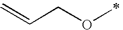 | Me | Ph | 3,4 diClPh | 497.1 (M + 1) |
| 128 | | | 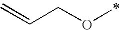 | Me | Ph | 3 N₃Ph | 470.2 (M + 1) |
| 129 | 4.58E−06 ± 1.59E−06 | 150.08 ± 9.89 | 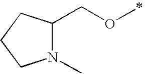 | Me | Ph | 3,4 diClPh | 556.0 (M + 1) |
| 130 | | | H₂N(CH₂)₂O | Me | Ph | 3 N₃Ph | 475.2 (M + 1) |
| 131 | 2.90E−06 ± | 158.97 ± | MeO | tBu | Ph | 3,4 diClPh | 515 (M + 1) |
| | 2.22E−06 | 9.43 | | | | | |
| 132 | 6.67E−04 ± | 113.43 ± | MeO | iPr | Thiophene | 3,4 diClPh | 491 (M + 1) |
| | 5.77E−05 | 15.43 | | | | | |
| 133 | 7.15E−08 ± | 180.24 ± | MeO | Cyclopropyl | Ph | 3,4 diClPh | 499 (M + 1) |
| | 1.87E−08 | 12.72 | | | | | |
| 134 | 1.00E−03 ± | 98.33 ± | MeO | iPr | 3 NO₂Ph | 3,4 diClPh | 546 (M + 1) |
| | 1.50E−11 | 4.08 | | | | | |
| 135 | 1.18E−07 ± | 186.32 ± | iPrO | Me | Ph | 3,4 diClPh | 501 (M + 1) |
| | 8.91E−08 | 21.26 | | | | | |
| 136 | | | MeO | Et | Ph | 3,4 diClPh | 487 (M + 1) |
| 137 | 1.56E−08 ± | 213.90 ± | MeO | Et | 2,6 diFPh | 3,4 diClPh | 523 (M + 1) |
| | 2.21E−09 | 32.41 | | | | | |
| 138 | 2.28E−08 ± | 271.13 ± | MeO | 2 FPh | 2 FPh | 3,4 diClPh | 505 (M + 1) |
| | 2.66E−08 | 44.37 | | | | | |
| 139 | 6.91E−09 ± | 281.08 ± | MeO | Et | Ph | 3 NO₂Ph | 464 (M + 1) |
| | 8.72E−09 | 41.60 | | | | | |
| 140 | 2.42E−09 ± | 266.63 ± | MeO | Et | 2 FPh | 3 NO₂Ph | 482 (M + 1) |
| | 1.89E−09 | 44.23 | | | | | |
| 141 | 1.43E−08 ± | 261.69 ± | MeO | Et | 2,6 diFPh | 3 NO₂Ph | 500 (M + 1) |
| | 4.97E−09 | 51.29 | | | | | |
| 142 | 6.78E−09 ± | 194.32 ± | EtO | Et | 2 FPh | 3,4 diClPh | 519.1 (M + 1) |
| | 1.90E−09 | 10.62 | | | | | |
| 143 | 5.24E−09 ± | 290.53 ± | MeO | Et | Ph | 3 CNPh | 444 (M + 1) |
| | 5.37E−09 | 62.04 | | | | | |
| 144 | 6.33E−08 ± | 187.17 ± | MeO | Cyclopropyl | 2 FPh | 3,4 diClPh | 517 (M + 1) |
| | 2.07E−08 | 15.16 | | | | | |
| 145 | 1.23E−07 ± | 224.48 ± | EtO | Et | Ph | 2,4 diClPh | 501.1 (M + 1) |
| | 7.88E−08 | 15.64 | | | | | |
| 146 | 4.03E−09 ± | 213.49 ± | EtO | Et | Ph | 2,5 diClPh | 501.1 (M + 1) |
| | 2.72E−09 | 2.69 | | | | | |
| 147 | | | EtO | Et | Ph | 3,4 diFPh | 469.2 (M + 1) |
| 148 | | | EtO | Et | Ph | 3 CO₂Me Ph | 491.2 (M + 1) |
| 149 | 1.11E−08 ± | 207.18 ± | EtO | Et | Ph | 3,5 diCF₃ Ph | 569.1 (M + 1) |
| | 7.21E−09 | 15.46 | | | | | |
| 150 | 2.14E−08 ± | 228.28 ± | MeO | Et | 2 FPh | 3 Me, 4 ClPh | 485 (M + 1) |
| | 1.91E−08 | 13.02 | | | | | |
| 151 | 1.01E−08 ± | 273.61 ± | EtO | Et | Ph | 3,5 diFPh | 469.0 (M + 1) |
| | 3.98E−09 | 72.77 | | | | | |
| 152 | 2.91E−06 ± | 238.69 ± | EtO | Et | Ph | 3 CO₂H Ph | 477.1 (M + 1) |
| | 1.58E−06 | 31.98 | | | | | |
| 153 | 1.53E−08 ± | 234.66 ± | MeO | Et | 2,4 diFPh | 3,4 diClPh | 523 (M + 1) |
| | 1.58E−08 | 47.50 | | | | | |
| 154 | 3.03E−07 ± | 125.87 ± | MeO | CH₂CH₂Ph | 2 FPh | 3,4 diClPh | 581 (M + 1) |
| | 1.46E−07 | 4.56 | | | | | |
| 155 | 1.93E−08 ± | 224.86 ± | MeO | Et | 2 F, | 3,4 diClPh | 539 (M + 1) |
| | 8.42E−09 | 33.97 | | | 3 ClPh | | |
| 156 | 3.69E−09 ± | 243.78 ± | MeO | Et | 2 F, | 3 NO₂ Ph | 516 (M + 1) |
| | 2.02E−09 | 28.90 | | | 3 ClPh | | |
| 157 | 3,69E−09 ± | 283.64 ± | MeO | Et | 2 F, | 3 CN Ph | 596 (M + 1) |
| | 9.91E−10 | 40.74 | | | 3 ClPh | | |
| 158 | | | MeO | Et | 2 F, | 3 ClPh | 505 (M + 1) |
| | | | | | 3 ClPh | | |
| 159 | | | MeO | Cyclopropyl | 2 FPh | 3 ClPh | 483 (M + 1) |
| 160 | 7.54E−08 ± | 186.95 ± | cyclohexyloxy | Et | Ph | 3,4 diClPh | 555.3 (M + 1) |
| | 3.39E−08 | 37.20 | | | | | |
| 161 | 3.54E−08 ± | 232.35 ± | iBuO | Et | Ph | 3,4 diClPh | 529.3 (M + 1) |
| | 2.27E−08 | 51.94 | | | | | |
| 162 | 1.97E−07 ± | 183.22 ± | sec-butyloxy | Et | 2 FPh | 3,4 diClPh | 547 (M + 1) |
| | 6.02E−08 | 32.30 | | | | | |
| 163 | 1.42E−07 ± | 224.85 ± | sec-butyloxy | Et | 2 FPh | 3,4 diClPh | 547 (M + 1) |
| | 5.23E−08 | 48.91 | | | | | |
| 164 | 2.83E−09 ± | 279.90 ± | MeO | Et | 2 FPh | 3 CN Ph | 462 (M + 1) |
| | 1.96E−09 | 42.27 | | | | | |
| 165 | 1.20E−08 ± | 2.50.83 ± | sec-butyloxy | Et | 2 FPh | 3 CN Ph | 504 (M + 1) |
| | 3.53E−09 | 35.75 | | | | | |
| 166 | 3.33E−08 ± | 219.07 ± | sec-butyloxy | Et | 2-FPh | 3 CN Ph | 504 (M + 1) |
| | 1.25E−08 | 9.81 | | | | | |
| 167 | 1.11E−04 ± | 255.95 ± | MeO | Et | 2 FPh | 3 ClPh | 471 (M + 1) |
| | 3.33E−04 | 20.32 | | | | | |
| 168 | 7.12E−09 ± | 239.64 ± | EtO | Et | 2 F, 4 Pyr | 3,4 diClPh | 522.2 (M + 1) |
| | 4.88E−09 | 17.88 | | | | | |
| 169 | 4.95E−09 ± | 261.04 ± | EtO | Et | 2 Cl, | 3,4 diClPh | 538.2 (M + 1) |
| | 2.17E−09 | 40.37 | | | 4 Pyr | | |
| 170 | 1.89E−09 ± | 262.16 ± | MeO | Et | 3,5 di FPh | 3,4 diFPh | 491.0 (M + 1) |
| | 1.47E−09 | 29.54 | | | | | |
| 171 | 8.28E−10 ± | 2.96.79 ± | MeO | Et | 3,5 di FPh | 3,4 diClPh | 523.0 (M + 1) |
| | 3.84E−10 | 48.99 | | | | | |
| 172 | 4.78E−10 ± | 304.08 ± | MeO | Et | 3,5 di FPh | Ph | 455.1 (M + 1) |
| | 1.42E−10 | 48.54 | | | | | |
| 173 | 9.65E−09 ± | 257.43 ± | EtO | Et | 2 F, 4 Pyr | 3,4 diFPh | 488.3 (M + 1) |
| | 4.72E−09 | 33.23 | | | | | |
| 174 | 2.19E−08 ± | 263.57 ± | EtO | Et | 2 F, 4 Pyr | 3 Pyr | 453.1 (M + 1) |
| | 4.92E−09 | 28.70 | | | | | |
| 175 | 1.83E−10 ± | 289.91 ± | MeO | Et | 2 F, 4 Pyr | 3 ClPh | 472 (M + 1) |
| | 1.77E−10 | 34.38 | | | | | |
| 176 | 4.21E−08 ± | 218.25 ± | MeO | Cyclopropyl | 2 F, 4 Pyr | 3 ClPh | 484 (M + 1) |
| | 4.25E−08 | 18.98 | | | | | |
| 177 | 7.96E−07 ± | 162.75 ± | MeO | iPr | 2 FPh | 3-CN Ph | 476 (M + 1H) |
| | 2.65E−07 | 23.07 | | | | | |
| 178 | 2.98E−08 ± 3.24E−09 | 258.67 ± 45.31 | 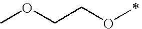 | Et | 2 FPh | 3- CN Ph | 506 (M + 1) |
| 179 | 9.65E−09 ± 4.03E−09 | 237.11 ± 19.59 | 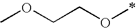 | Et | 2 FPh | 3 ClPh | 515 (M + 1) |
| 180 | 6.32E−08 ± 8.59E−09 | 279.68 ± 65.32 | 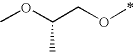 | Et | 2 FPh | 3-CN Ph | 520 (M + 1) |
| 181 | 2.10E−08 ± 1.11E−08 | 240.80 ± 29.53 | 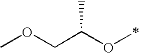 | Et | 2 FPh | 3 ClPh | 529 (M + 1) |
| 182 | 6.09E−08 ± 2.88E−08 | 251.54 ± 25.09 | 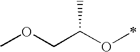 | Et | 2 FPh | 3 CN Ph | 520 (M + 1) |
| 183 | 4.35E−09 ± 1.51E−09 | 284.35 ± 38.37 | 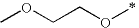 | Et | 2 F, 4 Pyr | 3 ClPh | 516 (M + 1) |
| 184 | 2.16E−08 ± 1.59E−08 | 268.39 ± 30.15 | 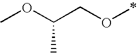 | Et | 2 F, 4 Pyr | 3 ClPh | 530 (M + 1) |
| 185 | 1.71E−06 ± 6.06E−07 | 172.89 ± 23.52 | 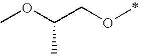 | Et | 2 FPh | 3 CN Ph | 520 (M + 1) |
| 186 | 5.89E−09 ± 1.06E−09 | 277.60 ± 32.53 | 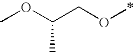 | Et | 2 F, 4 Pyr | 3 ClPh | 530 (M + 1) |
| 187 | 6.94E−07 ± 2.12E−07 | 162.93 ± 10.10 | 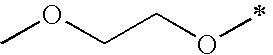 | Et | 2 FPh | 3 ClPh | 515 (M + 1) |
| 188 | 6.39E−06 ± 2.67E−06 | 300.00 ± 0.00 | 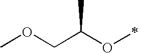 | Et | 2 F, 4 Pyr | 3 CN Ph | 521 (M + 1) |
| 189 | 8.66E−09 ± 7.46E−10 | 272.75 ± 38.41 | 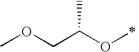 | Et | 2 F, 4 Pyr | 3 ClPh | 530 (M + 1) |
| 190 | 3.51E−06 ± 3.44E−06 | 266.06 ± 46.76 | 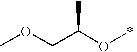 | Et | 2 F, 4 Pyr | 3 ClPh | 530 (M + 1) |
| 191 | 7.85E−09 ± 3.72E−09 | 263.67 ± 1.13 | 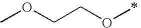 | Et | 2 F, 4 Pyr | 3 ClPh | 516 (M + 1) |
| 192 | 3.13E−08 ± 3.09E−08 | 237.07 ± 13.03 | 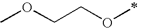 | Et | 2 F, 4 Pyr | 3 CN Ph | 507 (M + 1) |
| 193 | 7.76E−08 ± 5.91E−08 | 230.28 ± 6.46 | 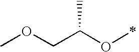 | Et | 2 FPh | 3 CN Ph | 520 (M + 1) |
| 194 | 2.47E−07 ± | 188.67 ± | EtO | Et | Ph | 3 CH₂CO₂Me₂ 4 FPh | 523.4 (M + 1) |
| | 6.98E−08 | 14.41 | | | | | |

The following amides are similarly prepared from the appropriate keto-amide using similar methods:

TABLE 3

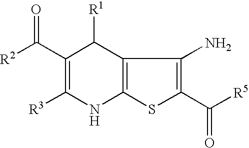

| Comp. | EC50, M | Max Plateau | R² | R³ | R¹ | R⁵ | MS Found |
|---|---|---|---|---|---|---|---|
| 195 | 3.37E−04 ± 5.75E−04 | 126.67 ± 23.09 | EtNH | Me | Ph | 3,4 diClPh | 486.0 (M + 1) |
| 196 | 3.34E−04 ± 5.77E−04 | 121.93 ± 25.12 | nBuNH | Me | Ph | 3,4 diClPh | 514.2 (M + 1) |

### Example H

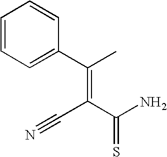

To a solution of acetophenone (3.6 g, 29.7 mmol) in benzene (210 ml) is added 2-cyanothioacetamide (3.06 g, 29.7 mmol), ammonium acetate (11.4 g, 148 mmol) and acetic acid (35 ml). The reaction mixture is heated at reflux for 5 hours. The mixture is cooled to room temperature, diluted with water, extracted with ethyl acetate, and washed with a saturated sodium chloride solution. The organics are dried over sodium sulfate, filtered and concentrated in vacuo. Purification by column chromatography (Ethyl acetate:Hexane=1:2) yields 2-cyano-3-phenyl-hut-2-enethioic acid amide (0.69 g) as a yellow solid (E/Z isomer). MS (M+1): 203.

### Example I

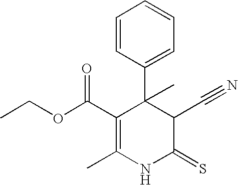

To a mixture of 2-cyano-3-phenyl-but-2-enethioic acid amide (273 mg, 1.35 mmol) in i-propanol (5 ml) is added ethyl acetoacetate (0.344 ml, 2.7 mmol) and 5.4 ml of potassium hydroxide solution (0.5 M in i-propanol, 2.7 mmol) at ambient temperature. The reaction mixture is heated at 50° C. for 5 hours, and stirred at ambient temperature for 1.5 hours. The mixture is quenched with 0.1 N aqueous hydrochloric acid, extracted with ethyl acetate, and washed with saturated sodium chloride solution. The organic extracts are dried over sodium sulfate, filtered and concentrated to give the crude product which is used for the next step without further purification. MS (M+1): 315.

### Example J

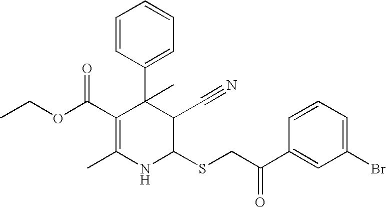

To a solution of 5-cyano-2,4-dimethyl-4-phenyl-6-thioxo-1,4,5,6-tetrahydro-pyridine-3-carboxylic acid ethyl ester (260 mg, 0.83 mmol) from example 1 and 2,3-dibromoacetophenone (278 mg, 1 mmol) in methanol (8 ml) was added piperidine (0.18 ml, 1.8 mmol) at ambient temperature. The reaction mixture is stirred at ambient temperature for 2 hours, diluted with water, and extracted twice with ethyl acetate. The extracts are washed with a saturated sodium chloride solution, dried over sodium sulfate, filtered and concentrated in vacuo. The residue is purified by flash chromatograph (Ethyl acetate:Hexane 1:1) to provide the product (130 mg) as a pale yellow solid. MS (M+2): 513.

### Example K

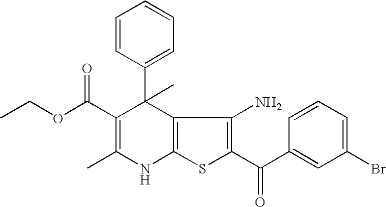

To a solution of 6-[2-(3-bromo-phenyl)-2-oxo-ethylsulfanyl]-5-cyano-2,4 dimethyl-4-phenyl-1,4 dihydro-pyridine-3-carboxylic acid ethyl ester from example I (87 mg, 0.17 mmol) in i-propanol (3 ml) was added 0.69 ml of potassium hydroxide (0.5 M in i-propanol solution) at ambient temperature. The reaction mixture is heated at 50° C. for 1 hour. The mixture is diluted with water, extracted with methylene chloride, washed with a saturated solution of sodium chloride, dried over sodium sulfate, filtered and concentrated in vacuo. The residue was purified by flash chromatograph (Ethyl acetate:Hexane=1:1) to provide the product (compound 197) as a yellow solid (31 mg). MS (M+2): 513.

### Example L

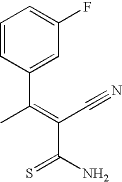

2-Cyanothioacetamide (6.73 g, 67.3 mmol) is added to a mixture of 3 fluoroacetophenone (8.85 g, 64.1 mmol), ammonium acetate (25.63 g, 333 mmol) and acetic acid (76.9 g, 1.28 mol) in benzene (170 ml) at room temperature. The reaction mixture is heated in a 101° C. oil bath with stirring for 5 hour. After cooling down to ambient temperature, the reaction mixture is diluted with ethyl acetate (300 ml). The organic layer washed with water (100 ml) and saturated NaHCO₃ solution (100 ml), dried over sodium sulfate and concentrated to dryness. The residue is purified by flash chromatography using 15-30% ethyl acetate in hexanes as eluting solvent to obtain 2-cyano-3-(3 fluoro-phenyl)-but-2-enethioic acid amide as a yellow solid (mixture E and Z isomers). MS (M+1): 221.1.

### Example M

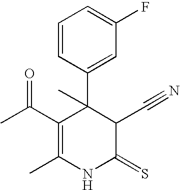

2-Cyano-3-(3 fluoro-phenyl)-but-2-enethioic acid amide (880 mg, 4.0 mmol) and 2,4-pentanedione are dissolved in isopropanol (20 ml) followed by the addition of 16 ml of 0.5 M potassium hydroxide in isopropanol. The reaction mixture is heated in a 75° C. oil bath with stirring for 7 hours. Upon cooling to ambient temperature, the reaction mixture is diluted with ethyl acetate (100 ml) and 1 N HCl (50 ml). The organic phase washed with brine (50 ml), dried over sodium sulfate, and concentrated to dryness. The residue is purified by flash chromatography using 20-45% ethyl acetate in hexanes as eluting solvent to obtain a brown viscous oil in which 5-acetyl-4-(3 fluoro-phenyl)-4,6 dimethyl-2-thioxo-1,2,3,4-tetrahydro-pyridine-3-carbonitrile was the majority.

### Example N

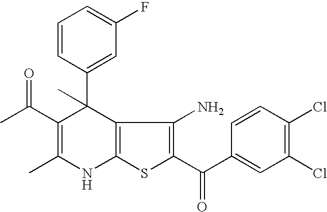

Crude 5-acetyl-4-(3 fluoro-phenyl)-4,6 dimethyl-2-thioxo-1,2,3,4-tetrahydro-pyridine-3-carbonitrile (380 mg, ca. 1.25 mmol) and 3,4 dichlorophenacyl bromide (670 mg, 2.50 mmol) are dissolved in ethanol (10 ml) followed by the addition of piperidine (0.16 ml, 1.63 mmol) and potassium carbonate (345 mg, 2.50 mmol). The reaction mixture is stirred at ambient temperature for 14 h. The reaction mixture is then diluted with ethyl acetate (100 ml) and water (50 ml). The organic layer is washed with brine (2×50 ml), dried over sodium sulfate, and concentrated to dryness. The residue is purified by preparative HPLC to obtain the desired product 1-[3-amino-2-(3,4 dichloro-benzoyl)-4-(3 fluoro-phenyl)-4,6-dimethyl-4,7 dihydro-thieno[2,3-b]pyridin-5-yl]-ethanone (compound 207) as a yellow solid. MS (M+1): 489.1.

The following are similarly prepared using the methods of examples H-K or L-N:

TABLE 4

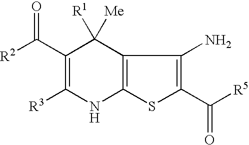

| Comp | EC50, M | Max Plateau | R² | R³ | R¹ | R⁵ | MS Found |
|---|---|---|---|---|---|---|---|
| 197 | 7.92E−07 ± 6.71E−07 | 159.55 ± 10.94 | EtO | Me | Ph | 3 BrPh | 513.1 (M + 2) |
| 198 | 1.00E−03 ± 0.00E+00 | 100.00 ± 0.00 | PhNH | Me | Ph | 3 ClPh | 560.2 (M + 1) |
| 199 | | | Me | Me | Ph | 3 ClPh | 483 (M + 1) |
| 200 | | | EtNH | Me | Ph | 2 ClPh | 512 (M + 1) |
| 201 | | | Me | Me | Ph | 3,4 diClPh | 471 (M + 1) |
| 202 | | | EtO | Me | 3 ClPh | 3,4 diClPh | 537 (M + 1) |
| 203 | | | EtO | Me | 3 ClPh | 3 NO₂Ph | 512.1 (M + 1) |
| 204 | | | EtO | Me | 4 ClPh | 3 NO₂Ph | 512.1 (M + 1) |
| 205 | | | EtO | Me | 4 ClPh | 3,4 diClPh | 537.1 (M + 1) |
| 206 | 9.36E−07 ± 3.04E−07 | 169.32 ± 23.07 | EtO | Me | Ph | Ph | 433 (M + 1) |
| 207 | 3.34E−06 ± 1.66E−06 | 148.73 ± 27.95 | Me | Me | 3 FPh | 3,4 diClPh | 489.1 (M + 1) |

The ethyl analog was also prepared.

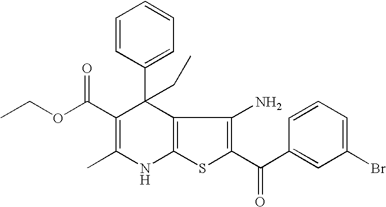

### Example O

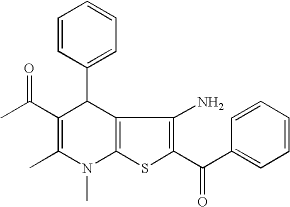

1-(3-Amino-2-benzoyl-6-methyl-4-phenyl-4,7 dihydro-thieno[2,3-b]pyridin-5-yl)-ethanone (388 mg, 1 mmol), methyl iodide (2 ml) and sodium methoxide (160 mg, 3 mmol) are combined in methanol (10 ml) and stirred in a sealed pressure vessel for 24 hours. A saturated solution of sodium chloride is added and the mixture acidified with citric acid. Ethyl acetate is added and the layers separated. The organic solvent is removed in vaccuo, the residues dissolved in ethyl acetate and washed with water. The combined organic layers are dried over magnesium sulfate, filtered and the solvents removed in vaccuo. The residue is purified by flash chromatography (silica gel, ethyl acetate:hexanes=1:3 to 1:1) to obtain the product, as a oil. This was crystallized from ethyl acetate, ether and hexanes to give the desired 1-(3-amino-2-benzoyl-6,7 dimethyl-4-phenyl-4,7 dihydro-thieno[2,3-b]pyridin-5-yl)-ethanone (compound 208) as a dark yellow solid.

TABLE 5

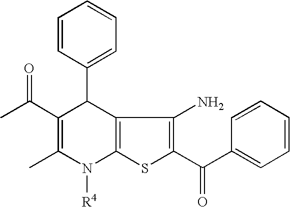

| Comp. | EC50, M | Max Plateau | R⁴ | MS Found |
|---|---|---|---|---|
| 208 | 3.43E−06 ± | 173.33 ± | Me | 403 (M + 1) |
| | 6.51E−07 | 11.55 | | |
| 209 | 3.93E−07 ± | 192.74 ± | MeO(CH₂)₂OCH₂— | 477 (M + 1) |
| | 6.63E−08 | 12.22 | | |

### Example P

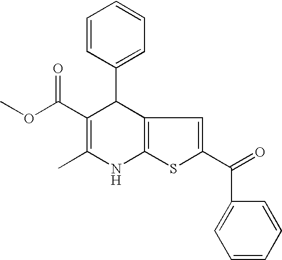

Methylacetoacetate (11.6 g, 100 mmol), ammonium acetate (8.5 g, 110 mmol), 2,2 dimethyl-1,3 dioxane-4,6 dione (14.4 g, 100 mmol) and benzaldehyde (10.6 g, 100 mmol) are combined in acetic acid (150 ml) and stirred at reflux for 10 hours. The mixture is poured onto crushed ice and the resulting precipitate collected by filtration and washed with water. The solid is recrystallized from ethanol to give 2-methyl-6-oxo-4-phenyl-1,4,5,6-tetrahydro-pyridine-3-carboxylic acid methyl ester. A solution of DMF (3.1 ml, 40 mmol) in chloroform (10 ml) is added dropwise, over 45 minutes, to phosphorus oxychloride (3.85 g, 40 mmol) at ambient temperature with stirring under a nitrogen atmosphere. The reaction is stirred for a further 30 minutes. 2-Methyl-6-oxo-4-phenyl-1,4,5,6-tetrahydro-pyridine-3-carboxylic acid methyl ester (2.45 g, 10 mmol) dissolved in methylene chloride (40 ml) is now added dropwise over 30 minutes. Stirring is continued for 16 hours at room temperature. A sodium acetate solution (40 g in 60 ml of water) is added slowly. The layers are separated, the aqueous layer is extracted with ethyl acetate, dried over magnesium sulfate, filtered and the solvents removed in vaccuo. The residue is purified by flash chromatography (silica gel, ethyl acetate:hexanes=1:2) to obtain the product as a oil. This was crystallized from ethyl acetate and hexanes to give an off-white solid. Thioacetic acid S-(2-oxo-2-phenyl-ethyl) ester (584 mg, 3 mmol), 6-chloro-5-formyl-2-methyl-4-phenyl-1,4 dihydro-pyridine-3-carboxylic acid methyl ester (581 mg, 2 mmol) and a 2 N solution of ammonium hydroxide in ethanol (2 ml) are combined with ethanol (20 ml) and stirred at ambient temperature for 20 hours. The solvent is removed in vaccuo, ethyl acetate and water are added and the layers separated. The organic layer is dried over magnesium sulfate, filtered and the solvents removed in vaccuo. The residue is purified by flash chromatography (silica gel, ethyl acetate:hexanes=1:2) to obtain the product, as a oil. This was crystallized from ethyl acetate and hexanes to give the desired 2-benzoyl-6-methyl-4-phenyl-4,7 dihydro-thieno[2,3-b]pyridine-5-carboxylic acid methyl ester.

### Example Q

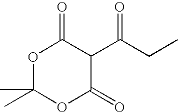

Meldrum's acid (14.4 g, 0.1 mol) is dissolved in dichloromethane (150 ml) and propionic anhydride (12.8 ml, 0.1 mol) added, followed by triethylamine (34.8 ml, 0.25 mol) and DMAP (1.22 g, 0.01 mol). After 30 minutes the reaction mixture is washed with 3×100 ml HCl. The organic layer dried over Na₂SO₄ and concentrated in vacuo. The residue is dried under high vacuum to give the 2,2 dimethyl-5-propionyl-[1,3]dioxane-4,6 dione as a white solid (16.25 g, 82%).

### Example R

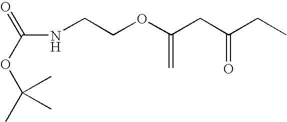

Boc-glycinol (1.108 g, 6.875 mmol) and 2,2 dimethyl-5-propionyl-[1,3]dioxane-4,6 dione (1.25 g, 6.25 mmol) are added to toluene (10 ml) in a 30 ml microwave vial equipped with a stir bar and the mixture irradiated at 120° C. for 20 minutes. The solution is concentrated in vacuo and the residue further dried under high vacuum overnight to afford 3-oxo-pentanoic acid 2-tert-butoxycarbonylamino-ethyl ester as an oil (1.62 g, 100%).

### Example S

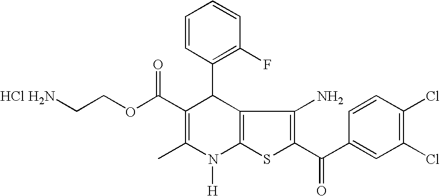

To a vial containing the Boc protected 3-amino-2-(3,4 dichloro-benzoyl)-4-(2 fluoro-phenyl)-6-methyl-4,7 dihydro-thieno[2,3-h]pyridine-5-carboxylic acid 2-amino-ethyl ester (compound 222, 10.3 mg, 0.0166 mmol) was added 1N HCl dioxane (330 μl) and the mixture stirred under nitrogen for 1.5 hour. The solution is concentrated in vacuo and the residue further dried under high vacuum over night to afford the HCl salt of 3-amino-2-(3,4 dichloro-benzoyl)-4-(2 fluoro-phenyl)-6-methyl-4,7 dihydro-thieno[2,3-b]pyridine-5-carboxylic acid 2-amino-ethyl ester (compound 226) as an orange powder (9.3 mg, 100%).

### Example T

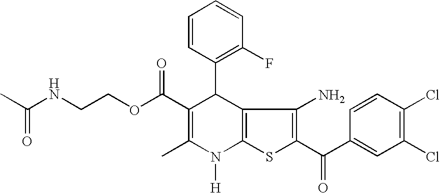

To a vial containing the HCl salt of 3-amino-2-(3,4 dichloro-benzoyl)-4-(2 fluoro-phenyl)-6-methyl-4,7 dihydro-thieno[2,3-b]pyridine-5-carboxylic acid 2-amino-ethyl ester (9.3 mg, 0.0166 mmol) is added DMF (100 μl) and 1M acetic anhydride in TH-F (17 μl, 0.0166 mmol), followed by triethylamine (4.2 mg, 0.042 mmol). The mixture is stirred at ambient temperature for 40 minutes. The solution is concentrated in vacuo and the residue taken up in ethyl acetate (2 ml) and washed with water (0.5 ml). The organic layer is dried over Na₂SO₄, filtered and concentrated in vacuo. The solid residue is further dried under high vacuum overnight to afford 3-amino-2-(3,4 dichloro-benzoyl)-4-(2 fluoro-phenyl)-6-methyl-4,7 dihydro-thieno[2,3-b]pyridine-5-carboxylic acid 2-acetylamino-ethyl ester (compound 225) as a yellow powder (9.2 mg, 98.5%).

### Example U

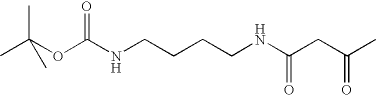

mono-BOC-1,4 diaminobutane (1.1 ml, 5.5 mmol) in CH₂Cl₂ (20 ml) is stirred at 0° C. and NHS-acetoacetate (1.0 g, 5 mmol) is added followed by triethylamine (0.77 ml, 5.5 mmol). After 2 hours, the reaction vessel is warmed to ambient temperature and stirred for 1 hour. CH₂Cl₂ (25 ml) is added and the product washed with 1N HCl (45 ml). The combined organic fractions are concentrated in vacuo to afford the clear-yellow [4-(3-oxo-butyrylamino)-butyl]-carbamic acid tert-butyl ester which was subsequently used without further purification.

The following long chain esters and amides are prepared using the methods of examples T and U:

TABLE 6

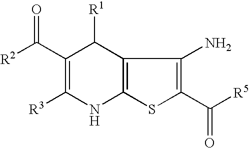

| Comp. | EC50, M | Max Plateau | R² | R³ | R¹ | R⁵ | MS Found |
|---|---|---|---|---|---|---|---|
| 210 | 9.56E−08 ± 2.90E−08 | 159.79 ± 4.77 | Fragment 1 below | Me | Ph | Ph | 534.3 (M + 1) |
| 211 | | | Fragment 1 below | Me | Ph | 3,4 diClPh | 602.3 (M + 1) |
| 212 | 1.00E−03 ± 0.00E+00 | 193.33 ± 161.66 | Fragment 2 below | Me | Ph | 3 N₃Ph | 701.4 (M + 1) |
| 213 | 3.26E−07 ± 3.01E−07 | 208.56 ± 23.45 | H₂N(CH₂)₂O | Me | Ph | 3,4 diClPh | 502 (M + 1) |
| 214 | 1.00E−03 ± 0.00E+00 | 100.00 ± 0.00 | AcHN(CH₂)₂O | Me | Ph | 3,4 diClPh | 544.1 M + 1) |
| 215 | 5.23E−06 ± 2.16E−06 | 156.60 ± 19.27 | Fragment 3 below | Me | Ph | 3,4 diClPh | 630.2 (M + 1) |
| 216 | 1.09E−06 ± 2.61E−07 | 160.98 ± 15.00 | Fragment 4 below | Me | Ph | 3 N₃Ph | 946.5 (M + 1) |
| 217 | 8.36E−04 ± 4.02E−04 | 105.00 ± 17.61 | Fragment 5 below | Me | Ph | 3 N₃Ph | 739.0 (M + 1) |
| 218 | 3.42E−04 ± 5.10E−04 | 130.81 ± 27.28 | Fragment 6 below | Me | Ph | Ph | 550.2 (M + 1) |
| 219 | 3.39E−04 ± 5.12E−04 | 120.38 ± 18.60 | Fragment 7 below | Me | Ph | Ph | 548.1 (M + 1) |
| 220 | | | Fragment 8 below | Me | Ph | Ph | 614 (M + 1) |
| 221 | 2.27E−08 ± 8.95E−09 | 156.50 ± 9.76 | H₂N(CH₂)₂O | Et | 2 FPh | 3,4 diClPh | 534.1 (M + 1) |
| 222 | 9.43E−08 ± 2.50E−08 | 128.75 ± 8.38 | tBuOCOHN(CH₂)₂O | Et | 2 FPh | 3,4 diClPh | 634.3 (M + 1) |
| 223 | 1.08E−08 ± 4.44E−09 | 213.11 ± 12.55 | tBuOCOHN(CH₂)₂O | Me | 2 FPh | 3,4 diClPh | 620.1 (M + 1) |
| 224 | | | AcHN(CH₂)₂O | Et | 2 FPh | 3,4 diClPh | 576.1 (M + 1) |
| 225 | | | AcHN(CH₂)₂O | Me | 2 FPh | 3,4 diClPh | 562.1 (M + 1) |
| 226 | | | H₂N(CH₂)₂O | Me | 2 FPh | 3,4-diClPh | 520.1 (M + 1) |

TABLE 7

| | |
|---|---|
| Fragment 1 | 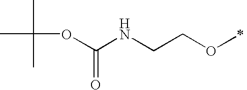 |
| Fragment 2 | 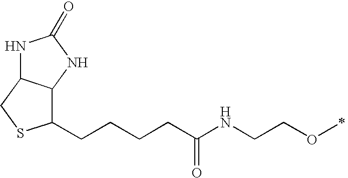 |
| Fragment 3 | 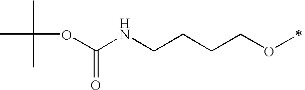 |
| Fragment 4 | 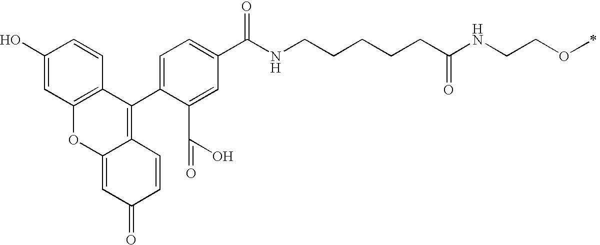 |
| Fragment 5 | 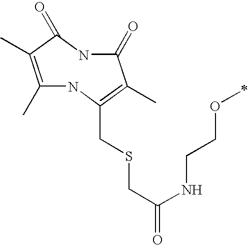 |
| Fragment 6 | 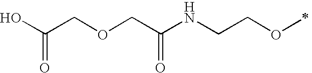 |
| Fragment 7 | 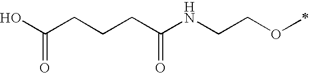 |
| Fragment 8 | 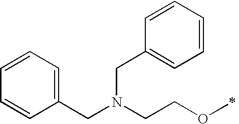 |

### Example V

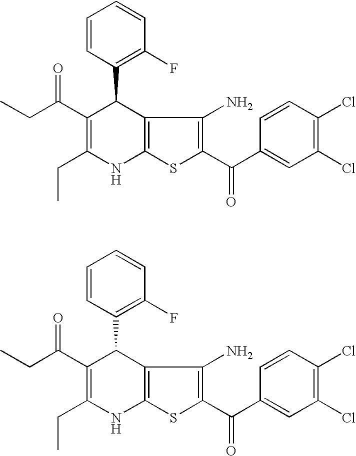

Racemic compound 39 is submitted to HPLC on a Chiralpak IA column (Daicel Chemical Industries, Inc.) using 15:85 2-propanol:heptane eluent at 15 ml/min. The enantiomers are separated with retention times of 15.0 min. and 18.0 min (active enantiomer). Solvents are removed to provide the separated enantiomers as yellow solid powders.

Enantiomer 1 specific rotation (methanol, 10.656 mg/cc)=−514.799 degrees.

Enantiomer 2 specific rotation (methanol, 8.042 mg/cc)=+506.462 degrees.

Similarly other racemic compounds are separated into their constitutive enantiomers using Chiralpak IA, AD, AD-H, AS, AS-H, or OD-RH columns (Daicel Chemical Industries) and mixtures of hexane, heptane, methanol, ethanol, 2-propanol, acetonitrile, dichloromethane, ethyl acetate, water, or other solvents, as known to one skilled in the art.

### Example W

In vitro activity of each compound is measured using the alpha-MyHC cytoblot process. Briefly, neonatal rat ventricular myocytes (NRVM) at 120,000 cells/ml in HyQ DME/High modified culture media supplemented with 10% charcoal/dextran treated FBS, 0.1% Nutridoma-SP, 1:100 MEM non-essential amino acids, 1:50 MEM amino acids solution w/o L-Gln, 1 mM sodium pyruvate and 1:100 P/S/G, are plated on gelatin coated sterile 384-well plates (Costar 3712) at 6,000 cells per well. Cells are incubated overnight at 37° C. in 5% CO₂ 100% humidity. Following overnight growth the media is changed to HyQ DME/High modified culture media supplemented with 0.3% Nutridoma-SP, 1:100 MEM non-essential amino acids, 1:50 MEM amino acids solution w/o L-Gln, 1 mM sodium pyruvate and 1:100 P/S/G. Cells are then dosed with serial dilutions of each compound in DMSO. Each concentration in the dose range is dosed in quadruplicate. Dosing is done in such a way so that the final DMSO concentration was 0.44%. Compound dilutions are 3-fold with the final top concentration of 10 uM. For potent compounds, dilutions series are started at 100 nM. Following dosing, the cells are incubated for 72 hours at 37° C. in 5% CO₂ 100% humidity and then processed with the cytoblot method. Briefly, cell media is aspirated and cell monolayers washed with PBS and fixed with 100% methanol for 30 minutes. Fixed cells are then blocked with PBS, 0.05% Tween-20, 1% BSA, for 1 hour. Following blocking, cells are incubated with primary alpha-MyHC-specific antibody (1:30 dilution of BA-G5 hybridoma conditioned media, ATCC HB276) for 1 hour. Following incubation with the primary antibody, cells are washed with PBS, 0.05% Tween-20, 1% BSA and incubated with secondary antibody (1:1000 dilution, goat-anti-mouse HRP, Southern Biotech, 1031-05) for 1 hour. Following incubation with the secondary antibody, wells are washed and incubated in SuperSignal, West Dura HRP-luminescence substrate for 30 seconds under shaking at room temperature. Luminescence is measured using a Packard Fusion plate reader. Relative Light Unit (RLU) values typically range between 2,000-3,000 for the low control (vehicle only), and between 8,000-10,000 for the high control (3 nM T3). RLU values are converted into percent fraction of low control and dose responses are fitted to a sigmoidal 4-parameter equation (XLFit, IDBS) where the EC₅₀ and Max Plateau values were extracted. EC₅₀ indicates the effective concentration that gives 50% of the maximum response. Max Plateau is the filtered maximum value determined by the dose response fit.

Tables 1-7 show in vitro activities of selected compounds.

FIG. 2 shows an example of dose response with compound 39.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,604,251.
U.S. Pat. No. 4,265,874.
U.S. Pat. No. 4,256,108.
U.S. Pat. No. 4,166,452.
Abraham et al., Mol. Med., 8(11):750-60, 2002.
Attaby et al., Phosphorous, Sulfur and Silicone and Related Elements, 119:1, 1996.
Bouvagnet et al., Basic Res. Cardiol., 84:91-102, 1989.
Bristow, Circulation, 101(5):558-569, 2000.
Bristow, Cardiology, 92:3-6, 1999.
Dipla et al., Circulation, 97(23):2316-2322, 1998.
Durand et al., Ann. Med., 27:311-317, 1995.
Eichhorn and Bristow, Circulation, 94:2285-2296, 1996.
Goodman & Gilman's The Pharmacological Basis Of Therapeutics, Hardman et al. ed., 10 ed., 32:853-860; 35:891-893, 2001.
Hajjar et al., Circulation, 86(6):1819-1826, 1992.
Huang, et al., FASEB J., 15:19-21, 2001.
Krauze and Duburs, Chemisty of Heterocyclic Compounds, 36:693, 2000.
Krauze et al., Khimiko-Farmatsevticheskii Zhurnal, 25:40, 1991.
Krauze et al., Khimiko-Farmatsevticheskii Zhurnal, 22:548, 955, 1988.
Krauze et al., Khimiya Geterotsiklicheskikh Soedinenii, 1694, 1984.
Krauze et al., Tetrahedron, 54:9161, 1998.
Lowes et al., J. Clin. Invest., 100(9):2315-2324, 1997.
Lowes et al., N. Engt J. Med., 346(18):1346-13467, 2002.
Marian and Roberts, Circulation, 92:1336-1347, 1995.
Miyata et al., Circ. Res., 86(4):386-390, 2000.
Nakao et al., J. Clin. Invest., 100(9):2362-2370, 1997.
Niimura et al., Circulation, 105(4):446-451, 2002.
Pagani et al., Circ. Res., 63(2):380-385, 1988.
PCT Patent Application No. WO 98/33791.
Reiser et al., Am. J. Physiol. Heart. Circ. Physiol, 280(4):H1814-H1820, 2001.
Remington's Pharmaceutical Sciences, 15^{th} ed., 1035-1038, 1570-1580, Mack Publishing Co., PA, 1980.
Schwartz et al., Circulation, 91:532-540, 1995.
Sharanin et al., Zhurnal Organicheskoi Khjiini, 22:2600, 1986.
Sharanin et al., Zhurnal Organicheskoi Kbimii, 21:683, 1985.
The Merck Index, O'Neil et al., ed., 13^{th} ed., 2001.
Thierfelder et al., Cell, 77:701-712, 1994.
Tirzite et al., Chem. Heterocyclic Compounds, 38:795, 2002.
Watkins et al., N. Engl. J. Med., 326: 1108-1114, 1992.
Yasna et al., Biochem. Pharmacol., 45:2527-2535, 1993.
Young et al., Handbook of Applied Therapeutics, 7.1-7.12 and 9.1-9.10, 1989.

We claim:

1. A compound or salt thereof, wherein the compound corresponds in structure to Formula I: 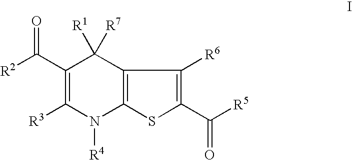
wherein:
R¹ is selected from the group consisting of phenyl, pyridinyl, cyclohexyl, benzodioxolyl, pyrazolyl, and naphthalenyl, wherein:
the phenyl, pyridinyl, cyclohexyl, pyrazolyl, and naphthalenyl are optionally substituted with one or more substituents independently selected from the group consisting of halogen, carboxy, alkoxy, hydroxyl, and alkyl, wherein:
the alkyl portions of such substituents optionally are substituted with a substituent selected from the group consisting of alkoxycarbonylamino, halogen, carboxy, alkylcarbonylamino, amino, alkylamino, and alkoxyamino, wherein:
the amino is optionally substituted with one or two substituents independently selected from the group consisting of alkyl, alkenyl, alkynyl, alkylcarbonyl, and alkoxycarbonyl;
R² is selected from the group consisting of monocyclic carbocyclyl, monocyclic heterocyclyl, naphthalenyl, alkyl, alkenyl, alkynyl, cycloalkyl, cycloalkyloxy, amino, alkoxy and phenyl, wherein:
the amino is optionally substituted with one or two substituents independently selected from the group consisting of alkyl, alkenyl, alkynyl, and cycloalkyl;
the alkoxy is optionally substituted with a substituent selected from the group consisting of alkyl, alkenyl, alkynyl, alkoxy, amino, monocyclic heterocyclyl, and monocyclic carbocyclyl, wherein:
the amino is optionally substituted with one or two substituents independently selected from the group consisting of carboxyalkoxyalkylcarbonyl, carboxyalkoxycarbonyl, alkylcarbonyl, alkoxycarbonyl, phenylalkyl, R⁸-alkylcarbonyl, and R⁸-carbonylaminoalkylcarbonyl, and
the heterocyclyl and carbocyclyl are optionally substituted with one or more substituents independently selected from the group consisting of alkyl, alkenyl, alkynyl, and alkoxy; and
R³ is selected from the group consisting of alkyl, alkenyl, alkynyl, phenylalkyl, alkoxyalkyl, haloalkyl, cycloalkyl, cycloalkenyl, phenyl, halophenyl, and alkoxy;
R⁴ is selected from the group consisting of hydrogen, alkyl, alkenyl, alkynyl, cycloalkyl, and alkoxyalkoxyalkyl;
R⁵ is selected from the group consisting of phenyl, pyridinyl, and benzodioxolyl, wherein:
the phenyl and pyridinyl are optionally ortho-mono-substituted, meta-mono-substituted, di-substituted, or ti-substituted with substituent(s) independently selected from the group consisting of halogen, azido, nitro, carboxy, cyano, alkyl, alkenyl, alkynyl, hydroxyl, alkoxy, thiol, alkylthio, haloalkyl, alkylcarbonyl, alkoxycarbonyl, alkoxycarbonylalkyl, and amino, wherein:
the amino is optionally substituted with one or two substituents independently selected from the group consisting of alkoxycarbonyl and alkylcarbonyl;
R⁶ is selected from the group consisting of hydrogen and amino;
R⁷ is selected from the group consisting of hydrogen, alkyl alkenyl, and alkynyl;
R⁸ is selected from the group consisting of 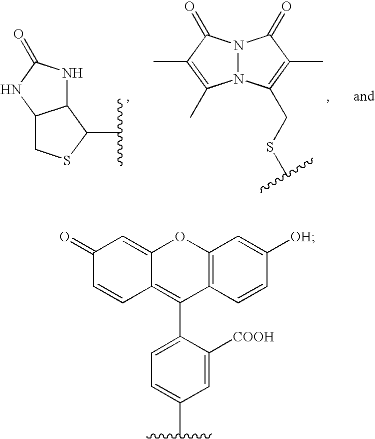
and
when compounds of Formula I are racemic, the following are excluded:

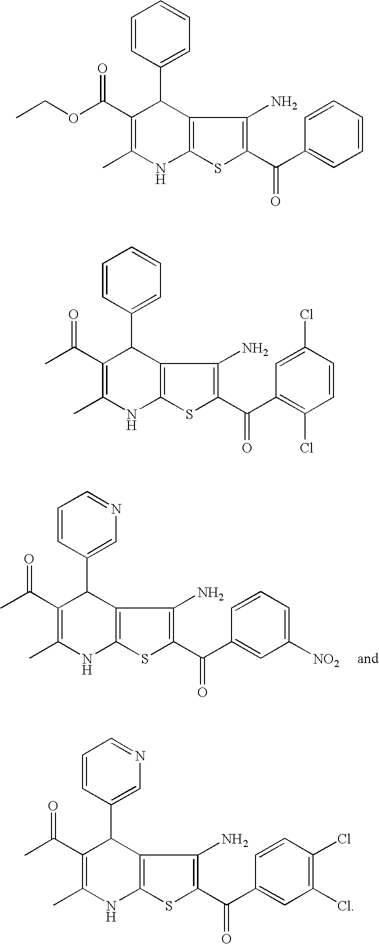

2. The compound or salt of claim 1, wherein the salt is a pharmaceutically acceptable salt.

3. The compound or salt of claim L, wherein the compound or salt is substantially pure.

4. The compound or salt of claim 1, wherein the compound or salt is at least about 80% pure.

5. The compound or salt of claim 1, wherein the compound or salt is in the form of one stereoisomer.

6. The compound or salt of claim 5, wherein the stereoisomer is substantially pure.

7. The compound or salt of claim 6, wherein the stereoisomer is at least about 80% pure.

8. The compound or salt of claim 5, wherein the stereoisomer is an enantiomer.

9. The compound or salt of claim 8, wherein the enantiomer is substantially pure.

10. The compound or salt of claim 9, wherein the enantiomer is at least about 80% pure.

11. The compound or salt of claim 1, wherein R⁴ is hydrogen, R⁷ is hydrogen and k6 is amino.

12. The compound or salt of claim 1, wherein R¹ is optionally substituted phenyl.

13. The compound or salt of claim 1, wherein R¹ is optionally substituted pyridinyl.

14. The compound or salt of claim 1, wherein R² is selected from the group consisting of alkyl, optionally substituted alkoxy, cycloalkyl, optionally substituted amino, and optionally substituted phenyl.

15. The compound or salt of claim 1, wherein R² is optionally substituted alkoxy.

16. The compound or salt of claim 1, wherein R² is alkyl.

17. The compound or salt of claim 1, wherein R³ is optionally substituted alkoxy.

18. The compound or salt of claim 1, wherein R³ is optionally substituted alkyl.

19. The compound or salt of claim 1, wherein R⁵ is optionally substituted phenyl.

20. The compound or salt of claim 1, wherein R⁵ is optionally substituted pyridinyl.

21. The compound or salt of claim 1, wherein R⁵ is phenyl.

22. The compound or salt of claim 1, wherein R⁵ is phenyl substituted with two independently selected substituents.

23. The compound or salt of claim 1, wherein R⁵ is pyridinyl.

24. The compound or salt of claim 1, wherein R⁵ is pyridinyl substituted with two independently selected substituents.

25. A compound or salt thereof, wherein the compound corresponds in structure to Formula II: 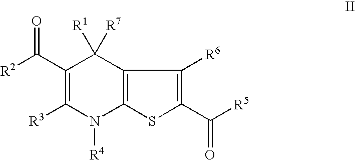
wherein:
R¹ is selected from the group consisting of monocyclic carbocyclyl, monocyclic heterocyclyl, naphthalenyl, and benzodioxolyl, wherein:
the carbocyclyl, heterocyclyl, and naphthalenyl are optionally substituted with one or more substituents independently selected from the group consisting of carboxy, alkyl, alkenyl, alkynyl, cycloalkyl, halogen, thiol, alkylthio, hydroxy, alkoxy, cyano, azido, nitro, and amino, wherein:
the alkyl portions of such substituents are optionally substituted with a substituent selected from the group consisting of thiol, alkoxy, halogen, and alkoxycarbonylamino; and
the amino is optionally substituted with one or two substituents selected from the group consisting of alkyl, alkenyl, alkynyl, alkylcarbonyl, and alkoxycarbonyl;
R¹ is selected from the group consisting of monocyclic carbocyclyl, monocyclic heterocyclyl, naphthalenyl, alkyl, alkenyl, alkynyl, alkoxy, cycloalkyloxy, and amino, wherein:
the amino is optionally substituted with one or two substituents independently selected from the group consisting of alkyl, alkenyl, alkynyl, and cycloalkyl; and
the alkoxy is optionally substituted with a substituent selected from the group consisting of alkyl, alkenyl, alkynyl, alkoxy, amino, N-morpholinyl, and N-methylpyrrolidinyl, wherein:
the amino is optionally substituted with one or two substituents independently selected from the group consisting of carboxyalkoxyalkylcarbonyl, carboxyalkoxycarbonyl, carboxyalkylcarbonyl, alkylcarbonyl, alkoxycarbonyl, phenylalkyl, R⁸-alkylcarbonyl, and R⁸-carbonylaminoalkylcarbonyl;
R³ is selected from the group consisting of C₂-C₈-alkyl, alkenyl, alkynyl, alkoxy, cycloalkyl, cycloalkenyl, and phenyl wherein:
the alkyl portions of such substituents are optionally substituted with a substituent selected from the group consisting of phenyl, alkoxy, and halogen; and
the phenyl is optionally substituted with one or more substituents independently selected from the group consisting of halogen, alkyl, alkenyl, alkynyl, alkoxy, and amino;
R⁴ is selected from the group consisting of hydrogen, alkyl, alkenyl, alkynyl, cycloalkyl, and alkoxyalkoxyalkyl;
R⁵ is selected from the group consisting of phenyl, pyridinyl, and benzodioxolyl, wherein:
the phenyl and pyridinyl are optionally substituted with one or more substituents independently selected from the group consisting of halogen, nitro, azido, carboxy, cyano, alkyl, alkenyl, alkynyl, hydroxy, alkoxy, thiol, alkylthio, haloalkyl, alkylcarbonyl, alkoxycarbonyl, and amino, wherein:
the amino is optionally substituted with one or two substituents independently selected from the group consisting of alkoxycarbonyl, alkylcarbonyl, alkoxycarbonylaminoalkylcarbonyl, and aminoalkylcarbonyl;
R⁶ is selected from the group consisting of hydrogen and amino;
R⁷ is selected from the group consisting of hydrogen, alkyl, alkenyl, and alkynyl;
R⁸ is selected from the group consisting of

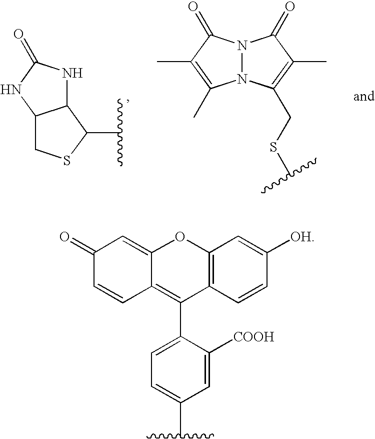

26. The compound or salt of claim 25, wherein R³ is C₂-C₈-alkyl.

27. The compound or salt of claim 26, wherein R³ is ethyl.

28. The compound or salt of claim 25, wherein:
R¹ is selected from the group consisting of optionally substituted phenyl and optionally substituted pyridinyl;
R² is selected from the group consisting of alkyl and optionally substituted alkoxy;
R³ is C₂-C₈-alkyl;
R⁴ is hydrogen;
R⁵ is selected from the group consisting of optionally substituted phenyl and optionally substituted pyridinyl;
R⁶ is amino; and
R⁷ is hydrogen.
